(19) United States
(12) Patent Application Publication
(10) Pub. No.: US 2026/0002123 A1
(43) Pub. Date: Jan. 1, 2026

(54) SINGLE-LUMEN ORGANOIDS AND METHODS OF PRODUCING THE SAME

(71) Applicant: President and Fellows of Harvard College, Cambridge, MA (US)

(72) Inventors: Ezekiel P. Benshirim, Cambridge, MA (US); Paola Arlotta, Cambridge, MA (US)

(21) Appl. No.: 19/253,197

(22) Filed: Jun. 27, 2025

Related U.S. Application Data
(60) Provisional application No. 63/665,120, filed on Jun. 27, 2024.

(51) Int. Cl. C12N 5/079

(52) U.S. Cl. CPC C12N 5/0618; C12N 2506/08; C12N 2513/00; C12N 2527/00

(57) ABSTRACT

Disclosed herein are single-lumen cortical organoids. Also disclosed herein are methods of inhibiting neurogenic differentiation and/or prolonging stem cell proliferation by increasing intraluminal pressure of a cortical organoid.

### RELATED APPLICATION(S)

This application claims the benefit of and priority to U.S. Provisional Application No. 63/665,120, filed Jun. 27, 2024. The entire teachings of the above application is incorporated herein by reference.

### GOVERNMENT SUPPORT

This invention was made with government support under MH123977 awarded by the National Institutes of Health. The government has certain rights in the invention.

### BACKGROUND OF THE INVENTION

Neuroscience research uses an estimated 23 million animals annually. Because human cerebral cortex development is both distinctive and experimentally inaccessible, animal models offer limited scope for understanding how the human brain acquires its distinctive functional abilities or modelling the causes of human-specific neurodevelopmental and neuropsychiatric diseases. Thus, concerns surrounding both translation and welfare have generated strong interest in using human organoids as an alternate model to study developmental neurobiology.

Stem cell-derived neural organoids can generate the same sequence of cortical cell types as in the embryo, enabling groundbreaking research on human neurodevelopmental conditions and toxicant exposures that alter cell fate and survival. Progenitors in organoids self-organize locally to form many small, polarized rosettes, reflecting but not fully recapitulating the single neural tube. Maturing organoids eventually lose this organization and suffer oxygen and nutrient stress throughout unstructured cores, compromising cellular fidelity at later ages. These problems motivate recent efforts to transplant organoids into animal brains for prolonged development.

### SUMMARY OF THE INVENTION

Neuroepithelial organoids were generated with a short-lived single initial lumen, which was pressurized with fluid for up to three months. In preliminary results, pressure dramatically improves tissue architecture, preserves biomimetic radial organization for longer than any existing protocol, and supports formation of single-rosette organoids over 1 mm in diameter. Inflated organoids show more uniform neural induction than controls, with a single continuous ventricular zone and uniform radial glial scaffold spanning the entire tissue, as in the embryonic cortex. Surprisingly, inflation also shows long-term effects on neural differentiation, demonstrating the importance of hydrostatic pressure for brain development and bioengineering.

Disclosed herein are methods of controlling neurogenic differentiation of an organoid, e.g., a developing organoid. In some embodiments, the methods include modifying the intraluminal pressure of an organoid, thereby controlling the neurogenic differentiation of the organoid.

In some embodiments, the organoid is a neuroepithelial organoid and/or a cortical organoid. In some embodiments, the intraluminal pressure of the organoid is increased by microinjecting fluid into one or more lumens of the organoid, thereby producing an inflated organoid. In some embodiments, the fluid is selected from the group consisting of: silicone oil, hydroxyfluoroether, or a solution of hyaluronic acid in saline.

In some embodiments, the fluid is injected into the lumen every 2 to 5 days for a period of up to 45 days. In some embodiments, the fluid is injected into the lumen every 2 to 5 days for a period of up to 90 days. In some embodiments, the fluid is injected into the lumen at a time point before rosettes appear in the organoid.

In some embodiments, increasing intraluminal pressure in the organoid inhibits neurogenic differentiation. In some embodiments, increasing intraluminal pressure in the organoid promotes or prolongs stem cell proliferation.

In some embodiments, the inflated organoid exhibits one or more of formation of single-rosette neural organoids greater than 500 μm in diameter, delayed appearance of ectopic additional rosettes, and stable maintenance of single-rosette organoids for up to 50 days. In some embodiments, the inflated organoid exhibits one or more of uniform biomimetic radial organization, improved quality and longevity of tissue architecture, uniform neural induction, having a single continuous ventricular zone, having uniform radial glial scaffold spanning the entire tissue, and lack of hypoxia.

In some embodiments, the methods further include reducing intraluminal pressure of the inflated organoid, thereby producing a deflated organoid. The reduction of intraluminal pressure may result in the activation of neurogenic differentiation of the deflated organoid.

Also disclosed herein are inflated single-lumen cortical organoids. In some embodiments, the lumen of the cortical organoid is injected with a fluid to increase lumen pressure.

In some embodiments, the fluid is selected from the group consisting of: silicone oil, hydroxyfluoroether, or a solution of hyaluronic acid in saline. In some embodiments, the organoid maintains tissue architecture resembling that of the developing mammalian neocortex for up to 1.5 months or up to 3 months. In some embodiments, neurogenic differentiation of the organoid is inhibited.

In some embodiments, the organoid comprises inflated cortical progenitors enriched for genes associated with DNA replication. In some embodiments, the organoid comprises inflated projection neurons enriched in GO terms related to mature neuronal function. In some embodiments, the organoid exhibits one or more of: formation of single-rosette neural organoids greater than 500 μm in diameter, delayed appearance of ectopic additional rosettes, and stable maintenance of single-rosette organoids for up to 50 days.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1C demonstrate ectopic rosettes form within days in uninflated single-lumen organoids. FIG. 1A provides a diagram of SOSRS differentiation protocol, from Tidball et al. 2022 (above). My phase-contrast images of replicated cut monolayers at d4, reannealed spheroids at d7, and individually suspension-cultured SOSRS at d13 (below). Right: SOSRS area at 1, 4 and 7 days after cutting, measured by manual thresholding and automated particle analysis in ImageJ. FIG. 1B shows immunofluorescent staining of SOSRS for ZO-1 (apical junctions), lamin (basement membrane), and SOX2 (neural stem cells) at d5-12. Note appearance of small marginal rosettes (some outlined in white). Right, Immunofluorescent staining of SOSRS for FOXG1 (forebrain progenitors) and DAPI at d5-12. Scale bars, 100 μm. FIG. 1C shows quantification of radial fraction of rosette margin containing rosettes in organoids 2-13 days after cutting monolayer and transferring to basement membrane. Summarized with mean±95% CI.

FIGS. 2A-2D demonstrate artificial inflation sustains radial organization for over one month and improves uniformity of neural induction. FIG. 2A provides a diagram of iSLO protocol as adapted (above). The phase-contrast images of a single mineral oil-injected iSLO from d10 to d31 (below). FIG. 2B shows iSLO immunostaining at d35 shows biomimetic expression and organization of neuroepithelium (left) and early radial glial scaffold with DCX-positive newborn neurons migrating (right). FIG. 2C shows immunostaining comparing iSLOs to uninjected controls at 1 month, showing more even expression of SOX2 and lack of later progenitors (EMX1) and neurons (MAP2). FIG. 2D shows immunostaining comparing iSLO to uninjected control shows radial glial scaffold with breaching migrating neurons, with much more radial glial stem cells and less mature neurofilament expression than controls.

FIGS. 3A-3D demonstrate inflation reproducibly and reversibly retards neurogenic differentiation even with radial symmetry is lost. FIG. 3A provides a timecourse of neuroepithelial and differentiation immunostaining panels shows that iSLOs remain neuroepithelial and do not progress to neurogenesis while inflated for up to three months, even while losing single-rosette tissue architecture. FIG. 3B shows “half-inflated” organoid that lost pressure in the left side on d42 shows that pressure continues to maintain early progenitor identity selectively in pressurized tissue within the same organoid. FIG. 3C shows quantification of immunofluorescence signal in similar organoids having both a pressurized and depressurized region allows preliminary quantitative confirmation of the positive effect of inflation on neuroepithelial marker expression and negative effect on later markers. Mean and 95% CI shown. FIG. 3D provides an example of an organoid that underwent spontaneous rupture and subsequent differentiation, similar to an uninflated control rather than a consistently inflated iSLO from the same batch.

FIG. 4 shows SOSRS injected with HA at d10 form fewer peripheral rosettes than uninjected controls at d13.

FIG. 5 provides a schematic of key steps in the formation of the cortex.

FIG. 6 shows the differences in tissue architecture between the embryonic brain and conventional organoid models.

FIGS. 7A-7D demonstrate single-rosette structure is rapidly lost in uninflated organoids. FIG. 7A provides a diagram of single-rosette cortical organoid differentiation protocol, from Tidball et al. 2023 (above). The phase-contrast images of replicated cut monolayers at d4, reannealed spheroids at d7, and individually suspension-cultured SOSRS at d13 (below). Right: SOSRS area at 1, 4 and 7 days after cutting, measured by manual thresholding and automated particle analysis in ImageJ. FIG. 7B shows IHC of SOSRS for ZO-1 (apical junctions), laminin (basement membrane), and SOX2 (neural stem cells) at d5-12, 1-8 days after cutting. Note appearance of small marginal rosettes (examples outlined in white) 8 days after cutting. Right, Immunofluorescent labelling of SOSRS for FOXG1 (forebrain progenitors) and DAPI at d5-12. Scale bars, 100 μm. FIG. 7C provides quantification of radial fraction of rosette margin containing rosettes in organoids 2-13 days after cutting monolayer and transferring to basement membrane. Summarized with mean±95% CI. FIG. 7D shows manually measured aspect ratios of nuclei in the ventricumlar zone surrounding the central lumen and, at day 9, in additional marginal rosettes. Summarized with mean±95% CI. Increasing aspect ratio suggests increasingly dense nuclear compression leading up to stem cell delamination from the central rosettte.

FIGS. 8A-8D demonstrate injection of single-lumen cortical organoids prolongs biomimetic organization. FIG. 8A provides a schematic of organoid differentiation and injection protocol. FIG. 8B shows representative phase contrast brightfield timecourses of individual uninflated, HFE-inflated, and HA-inflated organoids between 0.5 and 2.5 months. FIG. 8C provides survival curves for organoid growth, where survival is defined as maintenance of single-lumen gross morphology without rupture, major outgrowths, or death/failure to grow. Survival curves shown with Kaplan-Meier estimation of 95% CI. Pooled curves from N=3 batches HFE-injected, N=1 batch HA-injected and oil-injected, and N=4 batches uninjected. FIG. 8D shows continuation of HA-injected timecourse from (FIG. 8B) showing epifluorescence microscopic visualization of fluorescein hyaluronate aqueous solution trapped inside organoid lumen.

FIGS. 9A-9H demonstrate development and refinement of inflated single-lumen organoid protocol. FIG. 9A shows one-time injection of HA solution reduces radial fraction of organoid sections containing rosettes (examples marked in white) after 3 days. Scale bars, 250 μm. rosettes (examples marked in white) after 3 days. Scale bars, 250 μm. FIG. 9B shows effect of single injection on tissue architecture is not significant after 7 days. Scale bars, 100 μm. FIG. 9C shows one-time injection of fluorescein hyaluronate solution allows visualization of injected lumen and injection entry side in overlaid brightfield and GFP channels. Scale bar, 200 μm. FIG. 9D provides representative images showing decay in observed intraluminal intensity of green fluorescent signal in injected organoids over 3 days. Scale bars, 400 μm. FIG. 9E shows quantification of integrated green signal in lumens from N=2 batches (one injected on day 6 of culture and one on day 7) shows rapid logarithmic decay of fluorescein signal, with decay constant of 40-50% per day. FIG. 9F provides representative image of organoid floating at top of liquid near wall of well due to repeated injection with silicon oil. Scale bar, 500 μm. FIG. 9G provides representative two-channel images of single-lumen organoids injected with fluorescein hyaluronate at two Scale bars, 1000 μm or 2000 μm (bottom right). FIG. 9H shows addition of dissolved Geltrex to culture media for 24 h before initial injection increases the yield of injected organoids that successfully retain an injected HFE drop overnight. X-axis shows different biological replicates (batches), and each point represents a dish (technical replicate) with at least n=50 initially injected organoids. Effect of Geltrex, p<0.005 (hierarchical mixed linear effects model accounting for batch, cell line, and amount of Geltrex added).

FIGS. 10A-10F demonstrate injection allows growth of large, symmetric organoids with biomimetic histology. FIG. 10A provides representative IHC images of uninjected control and inflated single-lumen cortical organoid at 35 days of culture, labelled for DAPI (blue), neural stem cell marker SOX2 (green), basement membrane protein laminin (yellow), and apical junction protein ZO-1 (magenta). Note presence of many small rosettes growing out into mass of extracellular matrix left over from initial cyst formation, and uneven expression of SOX2. White dashed outline represents manually drawn boundary of DAPI+ nuclei infiltration into gel mass. FIG. 10B shows quantification of lumen number per section, longest stretch of ZO1+ apical surface per organoid, and mean ratio of total apical surface length to organoid perimeter, as an index of overall epithelial organization. Organoids from d35 (top) and d50 (bottom). *, p<0.05; **, p<0.01; ***, p<0.005. FIG. 10C provides representative images of uninjected control and iSLCO at day 50, immunolabeled as in (FIG. 10A). FIG. 10D provides higher-resolution images of rosette wall organization from areas outlined in (FIG. 10C). FIG. 10E provides representative images of uninjected control and iSLCO at day 50, immunolabeled for radial glial intermediate filament nestin (green), newborn neuronal marker DCX (red), and more mature neuronal intermediate filament NF-H (blue). FIG. 10F provides higher-resolution images of radial glial74scaffold organization and radial migration of newborn neurons, from areas outlined in (FIG. 10E).

FIG. 11 shows histology of inflated and control organoids at later timepoints. High-magnification images in FIG. 12 showing, in the top row, iSLCOs and control organoids labelled for SOX2 (green), Laminin (yellow), and ZO1 (magenta); in the middle row, MAP2 (postmitotic neurons, green), EMX1 (cortical progenitors, red), and nestin (radial glia, magenta); and on the bottom row, Ki67 (G2/M-phase cycling nuclei, green) and cleaved caspase-3 (apoptotic nuclei, red). Scale bars, 250 μm.

FIGS. 12A-12B demonstrate inflation prolongs expression of neural stemness at the expense of neurogenic differentiation. FIG. 12A provides an array of IHC images of iSCLOs and control organoids labelled for SOX2 (green), Laminin (yellow), and ZO1 (magenta); MAP2 (postmitotic neurons, green), EMX1 (cortical progenitors, red), and nestin (radial glia, magenta); and Ki67 (G2/M-phase cycling nuclei, green) and cleaved caspase-3 (apoptotic nuclei, red). Whole-organoid views of the images from FIG. 11. Scale bars, 1 mm. FIG. 12B shows quantification of the proportion of nuclei positive for SOX2, Ki67, NeuN, and cleaved caspase-3 between 25 and 90 days of culture, showing prolonged stem cell identity and proliferation in inflated organoids and increased neurogenesis in uninjected controls.

FIGS. 13A-13E demonstrate deflation allows inflated organoids to progress to neurogenesis. FIG. 13A shows representative spontaneously-half-deflated organoids at day 48 illustrate that inflation-preserved SOX2+/ZO1+ neuroepithelial stem cells retain the ability to differentiate into DCX+ and NFH+ neurons upon loss of pressure-induced stretch. FIG. 13B shows a representative half-deflated organoid at day 73 with example manually traced inflated and deflated regions, showing increased MAP2 and EMX1 expression in the deflated region and increased nestin expression in the region that retained continued inflation. FIG. 13C provides quantification of differential expression of key markers in half-deflated organoids shows that loss of pressure is associated with significant reductions in neuronal density along with stem cell markers ZO1, SOX2, and nestin, significant increase in EMX1 expression, and insignificant increases in MAP2 and DCX expression. FIG. 13D provides representative images of organoids collected one week after a subset were randomly assigned to be experimentally deflated on day 42. Note loss of neuroepithelial organization in deflated organoids. Immunolabels: SOX2 (neural stem cells, green), laminin (basement membrane, yellow), ZO-1 (apical progenitors, magenta); DCX (newborn neurons, green) and EMX1 (cortical progenitor nuclei, red); MAP2 (postmitotic neurons, green), nestin (radial glia, magenta). FIG. 13E shows quantification of SOX2+ nuclei in uninjected, deflated, and injected organoids. SOX2+ nucleus proportion in deflated organoids is reduced to levels similar to uninjected controls. Difference between deflated and injected organoids is not significant (p=0.14). *, p<0.05.

FIGS. 14A-14D demonstrate individual organoids sequenced at day 49 contain specific dorsal forebrain lineages. FIG. 14A shows UMAP representation (left) and proportional cell type composition (right) of the eight organoids sequenced. Note that each organoid primarily contains a pure population of cells from either the cortical-excitatory or cortical hem-Cajal-Retzius lineages. FIG. 14B provides dataset split by organoid, showing bifurcation of organoid identity into relatively pure cortical or cortical hem lineages. FIG. 14C provides a schematic of human telencephalon in coronal section with cell types color-coded as in panels (FIG. 14A) and (FIG. 14B). FIG. 14D provides feature plots showing selected markers used to annotate the dataset.

FIGS. 15A-15G demonstrate progenitor and neuronal cell type specificity is enhanced in inflated organoids. FIG. 15A, left, shows expression of canonical marker genes for actively cycling cells in G2/M phase and S phase. Expression is higher in reinjected organoids than deflated organoids (Wilcoxon signed-rank test, p<0.05 for PCNA and p<0.0001 for all others), FIG. 15A, right, shows result of subclustering and manually annotating cell clusters as G1, G2/M, or S phase. Cell state proportion change between reinjected and deflated organoids not significant at p<0.05 level. FIG. 15B provides a volcano plot of genes upregulated in reinjected (right) or deflated (left) cells assigned to the “cortical progenitor” cluster. FIG. 15C shows Gene Ontology term enrichment in genes upregulated in inflated (top) or deflated cortical progenitors relative to cortical progenitors in the other treatment condition. FIG. 15D provides volcano plot as in (FIG. 15B) for cells annotated as projection neurons. FIG. 15E provides GO enrichment plots as in (FIG. 15C) for cells annotated as projection neurons. FIG. 15F shows Log 2 fold-change enrichment of genes enriched in cortical progenitors relative to projection neurons by at least 2-fold in the pooled dataset. X-axis shows enrichment in deflated progenitors relative to deflated neurons, while Y-axis shows enrichment in inflated progenitors relative to inflated neurons, showing systematic enhancement in progenitor specificity of expression (median FC 43% larger, Wilcoxon ranked sum test p<1e-15). FIG. 15G provides a similar analysis as in (FIG. 15F), but for genes enriched in projection neurons relative to progenitors (median FC 35% larger, Wilcoxon ranked sum test p<1e-15).

FIGS. 16A-16H demonstrate modulate score analysis of pathways associated with inflation and deflation. FIG. 16A provides module scores for the “YAP1_ChEA” gene set, consisting of 2212 target genes of the YAP1 transcription factor identified through functional chromatin accessibility studies, Obtained from the ChEA Transcription Factor dataset (Lachmann et al. 2010). FIG. 16B provides module scores for the Focal adhesion: PI3K-Akt-mTOR signaling pathway (WP3932), consisting of 346 genes involved in signal transduction from focal adhesions through mTOR activity. Obtained from gsea-msigdb.org (Liberzon, A. Subramanian, et al. 2011). FIG. 16C provides module scores for the Hallmark Glycolysis gene set, consisting of 200 genes whose expression distinctively indicates glycolysis and gluconeogenesis. Obtained from the Molecular Signatures Database (MSigDB) Hallmark collection of gene sets (Liberzon, Birger, et al. 2015). FIG. 16D provides module scores for the Hallmark Hypoxia gene set, consisting of 200 genes whose expression distinctively indicates response to hypoxia. Obtained from the Molecular Signatures Database (MSigDB) Hallmark collection of gene sets (Liberzon, Birger, et al. 2015). FIG. 16E provides module scores for the Hallmark Fatty Acid Metabolism gene set, consisting of 158 genes whose expression distinctively indicates fatty acid metabolism. Obtained from the Molecular Signatures Database (MSigDB) Hallmark collection of gene sets (Liberzon, Birger, et al. 2015). FIG. 16F provides module scores for the Hallmark Cholesterol Homeostasis gene set, consisting of 74 genes whose expression distinctively indicates cholesterol biosynthesis regulation. Obtained from the Molecular Signatures Database (MSigDB) Hallmark collection of gene sets (Liberzon, Birger, et al. 2015). FIG. 16G provides module scores for the Hallmark TGF beta Signaling gene set, consisting of 54 genes whose expression distinctively indicates response to TGFβ signaling. Obtained from the Molec-ular Signatures Database (MSigDB) Hallmark collection of gene sets (Liberzon, Birger, et al. 2015). FIG. 16H provides module scores for the Hallmark WNT Beta Catenin Signaling gene set, consisting of 42 genes whose expression distinctively indicates canonical Wnt-β-catinin signaling. Obtained from the Molecular Signatures Database (MSigDB) Hallmark collection of gene sets (Liberzon, Birger, et al. 2015). Throughout this figure, red and blue asterisks (top) indicate effect size (*, Cohen's D>0.2; **, Cohen's D>0.5; ***, Cohen's D>0.8). Modules where reinjection increases expression are marked in red, while effect size for modules higher in deflated organoids are in blue. Black asterisks (bottom) indicate adjusted Bonferroni-corrected significance (*, p<0.05; **, p<0.01; ***, p<0.001).

FIGS. 17A-17B demonstrate human cerebral cortex development. FIG. 17A shows the cerebral cortex is the largest, most complex, and most distinctive region of the human brain. Cortical development begins with the neural tube's pseudostratified neuroepithelium, which is apicobasally polarized with the apical side lining a central fluid-filled canal that will become the brain ventricles. FIG. 17B shows a zoomed in section of the anterior dorsal neural tube of the cerebral cortex from FIG. 17A. This section is where the cortex will develop and the progenitor nuclei concentrate in a zone adjacent to the ventricle.

FIG. 18 provides a developmental timeline demonstrating that as the cortex grows throughout prenatal development a pool of neural stem cells in the ventricular zone generates diverse neuronal and glial cell types in a stereotyped sequence, starting with early-born deep neurons and progressing to more superficial layers. Compared to mouse models, human corticogenesis has a much longer timeline resulting in a thicker cortex with disproportionately more late-born, upper-layer projection neurons. Many questions remain about how human neural progenitors proliferate and differentiate; specifically, it is not known what “timekeeping mechanism” controls changes in progenitor fate potential along the “time” axis.

FIG. 19 demonstrates the development of human brain organoids. A population of pluripotent stem cells, which are typically dissociated, aggregated in 3D and cultured in neural differentiation media to yield neural progenitors followed by neuroglial progeny over a culture period of many months. Organoids are shown to recapitulate the birth of all major forebrain-derived cell lineages in the appropriate sequence. This offers excellent opportunities for studying conditions that affect specific cell types during brain development, but organoids still have limitations, notability in tissue architecture.

FIG. 20 shows that organoids can model human brain development, but do not model full tissue architecture. In particular, organoid progenitors to associate with each other, but in many radially symmetric rosettes instead of a single continuous germinal zone as in the embryo. Neurons that migrate outwards from these rosettes cannot form organized tissue architecture in vivo. Furthermore, beyond the oxygen diffusion limit, these organoids develop an unhealthy core that shows signs of hypoxia and nutrient stress. An initial hypothesis is that bioengineering can be used to recapitulate more aspects of early embryonic tissue architecture to help achieve better tissue organization later on. Work from another lab has made progress towards this goal, achieving organoids with reproducible initial single-lumen patterning, that more closely resembles the organization of the early embryonic neural tube. However, when cultured longer, these models develop additional rosettes within just a week after forming a single rosette. This protocol will be built on to sustain biomimetic tissue architecture for longer in order to gain insights into how spatial niches influence cortical progenitor stemness and fate decisions.

FIG. 21 shows human embryonic brains are inflated by pressurized fluid. More specifically, human embryonic brains are more fluid-filled cavity than tissue for most of the first two months of development. This inflation does not spontaneously occur in organoids.

FIG. 22 shows that intraluminal pressure is necessary for proper development of normal cortical tissue architecture. When embryonic cerebrospinal fluid is experimentally drained from chick embryos just before neurogenesis, the tissue becomes disorganized and folded like an organoid. Similarly, human neural tube malformations in which the anterior neuropore fails to close result in fetal brains filled with rosette structures very similar to those seen in human organoids. Because intraluminal pressure is clearly necessary for proper development of normal cortical tissue architecture, it was sought to add it back to single-lumen human cortical organoids to test to what extent it is also sufficient.

FIGS. 23A-23B demonstrate the manual injection of fluid into single-lumen organoids. FIG. 23A provides a schematic for manually injecting fluid into single-lumen organoids. A previous protocol was adapted for growing single-lumen neural organoids, which begins with neural induction of a monolayer of iPSCs, followed by cutting the monolayer into small squares that reorganize on a bed of GelTrex. To add internal pressure, fluid was manually injected into the organoid lumens prior to the development of rosettes and again every 3-5 days thereafter. Three different fluids were injected: a biomimetic hyaluronic acid solution, bioinert silicone oil, and hydroxyfluoroether. FIG. 23B shows the injection process of a fluid into the organoid. Brightfield imaging shows that oil-injected organoids grow over time. In addition, organoids can be injected with an aqueous solution of HA, which may be combined with other water-soluble signaling factors.

FIG. 24 shows pressure sustains biomimetic tissue architecture. After 35 days, uninjected control organoids had the typical multiple rosettes, each resembling an individual neural tube with the ventricular surface in magenta, and a neural stem cell marker in green, somewhat evenly expressed between rosettes. In contrast, a symmetric injected organoid more closely resembles the embryonic cortex. There is a single continuous ventricular zone of neural stem cells evenly expressing their marker SOX2, and a continuous ventricular surface with progenitor endfeet outlined by apical junctions. In addition, there is no core to develop hypoxia. These are the largest and oldest cortical organoids that have been reported with such a symmetric, apicobasally polarized, biomimetic tissue architecture. Staining for the radial glial scaffold that organizes radial migration, in green, similarly shows a more biomimetic, radially symmetric structure, with newborn neurons in magenta migrating outwards.

FIGS. 25A-25C demonstrates intraluminal pressure preserves progenitor stemness. FIG. 25A shows the assessment of the effect of intraluminal pressure on tissue over an extended period of time. The organoids were continuously injected and were harvested at various timepoints up to 3 months. Beginning at about 1.5 months, inflated organoid tissue does begin to break up into multiple rosettes around a single large lumen. These results showed that adding pressure alone to an organoid is sufficient to maintain tissue architecture for much longer, but not long enough to make a perfect cortex. However, a different and interesting result was found from this panel: unexpectedly, intraluminal pressure continues to strongly affect progenitor differentiation even after rosettes form. While in controls, SOX2+ neural stem cells become a minority over time, in inflated organoids almost all nuclei are SOX2-positive as late as 3 months. This suggests that the application of intraluminal pressure is changing the state of the stem cells so that they favor maintenance of stemness over differentiation into neurons. FIG. 25B shows that in addition to marking the apical epithelial surface, ZO-1 (magenta) is a temporal marker, expressed in early neuroepithelial stem cells, but not in later progenitors or neurons. By d73 in uninjected controls ZO-1 is not present, but is still strongly expressed at 3 mo in inflated organoids. FIG. 25C provides stains for relatively early radial glial progenitors in magenta; later cortical progenitors in red; and postmitotic neurons in green. Controls more through these stages to be primarily neurons by 90 days. However, pressure preserves injected organoids in an early progenitor state, with few neurons except in peripheral outgrowths that were not exposed to intraluminal pressure. This points at the existence of a regulatory mechanism whereby the pressure in the neural tube could be tuned to tell progenitors when to maintain their stemness or differentiate.

FIGS. 26A-26C demonstrate stemness preservation is reversible and quantifiable. FIG. 26A provides brightfield images showing an organoid that remained inflated until harvest at 2.5 months, compared to one that spontaneously ruptured during reinjection at 1.5 months and remained uninflated for an additional month. Immunostaining shows that the inflated organoid has little neurogenesis, as expected, while the collapsed organoid has abundant neurons and cortical progenitors, much closer to a never-inflated control, perhaps a little delayed with more radial glial signal remaining. FIG. 26B demonstrates the quantification of the effects of pressure on the organoids. Two adjacent inflated organoids were fused together into one shortly after injection. Then the left side ruptured on day 44 and was without pressure for 4 days before the fused organoid was harvested. There was an outer layer of nuclei on the left side that stopped being green neural stem cells (the depressurized side). Newborn neurons (stained in green) are abundant throughout this half while being absent from the pressurized tissue in the same fused organoid. The relative expression of several markers was quantified in pressurized vs depressurized regions of organoids between 2 and 3 months, confirming that pressure increases expression of early neural stem cell makers while inhibiting expression of later cortical progenitor and neuronal markers. FIG. 26C provides a closer look at the last stain (FIG. 26B) to show how neurogenesis is mainly happening in the depressurized side, as well as a little bit where cells have migrated outside the stretched epithelial shell and started to pile up in an unstructured layer on the side. Together these results show that pressures pro-stemness effects are reversible. Pressure inhibits progenitors from making neurons, but does not remove their potential to do so, only the timing of when this occurs. It also confirms, again, that these are not simply biochemical effects of the injected fluids, since the depressurized side is in the same well and still in contact with residual fluid.

FIG. 27 shows that by manually pressurizing single-rosette human cortical brain organoids biomimetic single-lumen tissue architecture can be stabilized for over a month. Even after this period, intraluminal pressure has an unexpected role in maintaining progenitor stemness and inhibiting neurogenic differentiation, and this effect can be reversed upon loss of pressure. Together, these results show that at least in organoids, intraluminal pressure is a mechanism for determining the timing of neurogenesis in developing cortical tissue. This could be a tunable and readily evolvable mechanism for controlling developmental timing in embryonic neural development across species. Organoids may be analyzed before and after intentional deflation to explore how pressure affects the maturation and neurogenic potential of cortical progenitors to gain hints about the nature of the timekeeping mechanism.

### DETAILED DESCRIPTION OF THE INVENTION

It is shown that regardless of the fluid used, lumen inflation dramatically improves organoid tissue architecture, extending the maintenance of single-lumen morphology from an average of two weeks to up to three months, while promoting continued neural stem cell division and inhibiting the birth of new neurons (neurogenesis). Experimental deflation of organoids reverses this effect, allowing rapid neuronal differentiation, which is characterized using single-cell RNA sequencing. Transcriptomic analysis of experimentally inflated or deflated organoids shows that these organoids match the cell types present in the original uninflated protocol, reveals improved segregation of neural progenitor and neuronal identity, and sheds light on the molecular pathways affected by inflation. These results demonstrate that intraluminal pressure is a potent regulator of the neurogenic switch in an in vitro model of human cortical development, with potential implications for understanding cortical evolution and neurodevelopmental disease.

Disclosed herein are methods of controlling neurogenic differentiation of an organoid. In some embodiments, the organoid is a neuroepithelial organoid. In some embodiments, the organoid is a cortical organoid. In certain aspects, the organoid has either a cortical or cortical hem lineage. In some embodiments, an organoid is generated from pluripotent stem cells, e.g., human induced pluripotent stem cells (hiPSCs). Organoids may be mammalian organoids, e.g., human or non-human organoids.

In some aspects, organoids are obtained using a protocol described in Tidball et al. 2023, incorporated herein by reference. In some embodiments, the Tidball protocol is modified. For example, the Tidball protocol may be modified to prolong the initial neural induction phase for up to 7 days and/or seeding at a lower density. In some embodiments, single-rosette organoids or single-lumen organoids are obtained, e.g., from hiPSCs. The inflated organoids may maintain tissue architecture that closely resembles that of the developing mammalian neocortex for up to 1.5 months. In some aspects, the inflated organoids have single ventricle-like structures reaching over a millimeter in diameter.

In some embodiments, the intraluminal pressure of an organoid, e.g., a single-rosette organoid, is modified. Modifying the intraluminal pressure of the organoid may result in controlling the neurogenic differentiation of the organoid. In addition, modifying the intraluminal pressure of the organoid may result in controlling stem cell proliferation. For example, the intraluminal pressure of the organoid may be increased by inflating the organoid. The organoid may be inflated by injecting, e.g., microinjecting, a fluid into a lumen of the organoid. In some embodiments, an organoid, such as an inflated organoid, is deflated. An inflated organoid may be deflated intentionally or unintentionally.

In some embodiments, the fluid injected into the lumen is a silicon oil, hydroxyfluoroether, or a solution of hyaluronic acid. In some embodiments, about 2 to about 3 nl of fluid was injected into the organoid. After injection, the organoid may be cultured for at least 24 hours. In some embodiments, injected or inflated organoids are cultured for at least 5, 10, 15, 20, 25, 30, 35, 40, 45, 50, 60, 65, 70, 75, 80, 85, 90, or more days. In some embodiments, the organoids were reinjected with fluid every 2 to 5 days. In certain aspects, the organoids were reinjected with additional fluid when the organoid shell thickness exceeded a predefined value, e.g., 100 μm until day 30 and 200 μm until day 60. After 60 days, an organoid may be reinjected with fluid when the primary shell tissue becomes slack rather than turgid. In some embodiments, fluid is injected into the lumen before rosettes appear in the organoid. In some embodiments, injected or inflated organoids were monitored to confirm they had a large, visible central lumen with no other macroscopically apparent lumens, there was no rupture and/or release of injected fluid, and the estimated combined size of any disorganized outgrowths, added together, did not exceed the size of the central symmetric shell.

In some embodiments, inflated organoids are deflated. For example, inflated organoids may become deflated by not reinjecting organoids with fluid over a period of time. Alternatively, inflated organoids may rupture during or after the process of inflation, e.g., microinjecting the organoid with fluid. By deflating an organoid, e.g., an inflated organoid, neural differentiation may restart.

Inflated organoids may exhibit one or more characteristics that distinguish them from organoids produced utilizing a similar protocol, but that are not inflated. For example, inflated organoids comprise less lumens per section and/or longer maximum stretches of apical surface. The inflated organoids exhibit differences from non-inflated organoids at 30 days post inflation. In some embodiments, the inflated organoids have a ventricular zone with a thickness of about 50, 60, 70, 80, 90, 100, or 110 μm, or more specifically about 80±20 μm. In some embodiments, the inflated organoids have an early marginal zone, e.g., having SOX2-negative nuclei, with a thickness of 10, 20, 30, 40, or 50 μm, or more specifically about 30±10 μm. The inflated organoids may exhibit significant improvements in the survival and extent of biomimetic neuroepithelial organization, e.g., for at least one month.

In some embodiments, inflated organoids continue to exhibit improvements in the formation and/or maintenance of the radial glial scaffold, e.g., as compared to uninflated controls at 50 days post inflation. For example, the inflated organoids comprise less lumen per section, exhibit longer stretches of neuroepithelial apical surface, and have a higher neuroepithelial index. In some embodiments, inflated organoids exhibit relatively dense and/or well-organized ventricular zones. In some aspects, the inflated organoids expressed SOX2 uniformly in the ventricular zones and/or exhibited a thin layer of SOX2-negative nuclei in a marginal zone. In some embodiments, ZO-1 is expressed at apical surfaces of the inflated organoids. In some embodiments, the inflated organoids have a ventricular zone with a thickness of about 80, 90, 100, 110, 120, 130, 140, 150, 160, 170, or 180 μm, or more specifically about 130±40 μm. In some embodiments, the inflated organoids have one or more zones (e.g., putative marginal, intermediate, and/or preplate zones) located between the ventricular zone and the surface of the inflated organoid, and those one or more zones have a combined thickness of 50, 60, 70, 80, 90, 100, 110, 120, 130, 140, 150, 160, 170, 180, 190, 200, 210, 220, 230, or 240 μm. In some embodiments, the inflated organoids exhibited a dense array of nestin-positive fibers oriented parallel to each other and/or perpendicular to the lumen surface, e.g., resembling the embryonic radial glial scaffold. In some embodiments, the inflated organoids exhibit DCX-positive cells accumulating at the basal surface.

In some embodiments, inflated organoids comprise a single-rosette neural organoid. In some embodiments, inflated organoids comprise a single-rosette neural organoids having a diameter of at least 250, 300, 350, 400, 450, 500, 550, 600, 650, 700, 750, 800, 850, 900, 950, or 1000 μm diameter. In some embodiments, inflated organoids exhibit delayed appearance of ectopic rosettes, e.g., of additional ectopic rosettes. In some embodiments, inflated organoids exhibit stable maintenance of the organoid, e.g., a single-rosette organoid, for up to 25, 30, 35, 40, 45, 50, 55, 60, 65, 70, 75, 80, 85, 90, or 95, or 100 days.

In some embodiments, inflated organoids exhibit biomimetic radial organization, e.g., uniform biomimetic radial organization. In some embodiments, inflated organoids comprise a single continuous ventricular zone. In some embodiments, inflated organoids comprise uniform radial glial scaffold, e.g., spanning the entire tissue. In some embodiments, inflated organoids exhibit a lack of hypoxia.

Inflated or injected organoids may exhibit decreased or delayed neuronal differentiation, e.g., as compared to control organoids (i.e., organoids that are not inflated). In some aspects, inflated organoids reside in a mitotic state. In some embodiments, inflated organoids exhibit or show a neuroepithelial identity over an extended period in culture, such as for 30, 45, 60, 75, or 90 days in culture. Inflation was shown to enhance and/or prolong the specificity of neuron and progenitor cell classes, thereby reducing overlap between these identities within individual cells.

In some embodiments, inflated organoids express SOX2, Ki67 and/or ZO-1. In some aspects, the expression of SOX2, Ki67 and/or ZO-1 is higher in inflated organoids than in non-inflated organoids after the same time period in culture. In some embodiments, inflated organoids exhibit increased expression of canonical markers of the G2/M phase (e.g., MKI67) and/or of the S phase (e.g., PCNA), e.g., as compared to uninflated or deflated organoids. In some embodiments, inflated organoids exhibit increased expression of one or more cell stress genes, such as genes related to hypoxia and glycolysis. More specifically, inflated progenitors exhibit increased expression of one or more cell stress genes. In some embodiments, inflated organoids exhibited increased expression of one or more metabolic genes relating to fatty acid metabolism, such as genes related to fatty acid metabolism and cholesterol homeostasis. More specifically, inflated progenitors exhibit increased expression of one or more metabolic genes.

In some embodiments, inflated organoids exhibit higher expression of genes associated with DNA replication and/or other housekeeping genes, e.g., as compared to uninflated organoids. In some embodiments, inflated projection neurons are strongly enriched in GO terms related to mature neuronal function. For example, inflated projection neurons are enriched in one or more the following GO terms: synaptic neurotransmitter release, transmembrane ion transport, and response to calcium signaling.

In some embodiments, neural differentiation is restarted upon deflation of an inflated organoid. For example, an inflated organoid that is deflated, e.g., intentionally or unintentionally, may resume neural differentiation after deflation. In some aspects, the loss of intraluminal pressure induces differentiation towards a neuron-like fate in cortical radial glia, e.g., at the expense of proliferation-related activity. In some embodiments, a deflated organoid may exhibit a decrease in expression of ZO1, SOX2, and nestin, e.g., as compared to an inflated organoid. In some embodiments, a deflated organoid exhibits increased expression of MAP2, e.g., as compared to an inflated organoid. In some embodiments, a deflated organoid exhibits increased expression of EMX1, e.g., as compared to an inflated organoid. In some embodiments, deflated organoids formed multiple, e.g., more than one, isolated rosettes. In some aspects, each rosette comprises an isolated ZO-1 positive focus. In some embodiments, deflated organoids exhibit increased expression of one or more genes associated with canonical Wnt and TGFβ signaling activity.

In some embodiments, deflated organoids exhibit higher expression of genes related to differentiation and function of mature neurons, e.g., as compared to inflated organoids. In some embodiments, deflated projection neurons are enriched in GO terms related to the proliferation and differentiation of neural stem cells. For example, deflated projection neurons are enriched in one or more the following GO terms: synaptic neurotransmitter release, transmembrane ion transport, and response to calcium signaling.

Inflated organoids may be used to model the morphogenesis of the cerebral cortex. In some embodiments, the inflated organoids model the morphogenesis of the developing cerebral cortex, e.g., the human cerebral cortex. In some embodiments, the inflated organoids model the intraluminal pressure in developing neural tubes. In some embodiments, inflated organoids are used to model cortical histogenesis. In some embodiments, the inflation and/or deflation of the organoids is adjusted to model that of the embryonic brain, e.g., the development of the embryonic brain. In some embodiments, the biological effects of embryonic cerebrospinal fluid (eCSF) is examined using organoids, e.g., inflating organoids with eCSF or an eCSF substitute. In some embodiments, inflated organoids are used to examine apicobasally-restricted signals on neural progenitors. In some embodiments, inflated organoids are used to examine the interaction of stem cell stress and tissue architecture. For example, the interaction of stem cell stress and tissue architecture and the resulting effect on the behavior of neural organoid cells during differentiation can be examined.

### EXAMPLES

### Example 1: Enhancing Tissue Architecture and Fidelity in Single-Lumen Human Brain Organoids Using Hydrostatic Pressure

### Summary:

There is widespread interest in modeling the human cerebral cortex in vitro to study neurological diseases that cause intense suffering, both in patients and lab animal models. Organoid models faithfully recapitulate early cortical differentiation at the single-cell level, but their lack of endogenous tissue architecture and bias towards earlier-born cell types currently limit their ability to replace animal models. Because the multiple ectopic lumens in cortical organoids resemble those in embryonic neural tubes with defective inflation, the inventors hypothesized that artificially inflating organoids will improve their morphogenesis. The inventors generated neuroepithelial organoids with a short-lived single initial lumen and pressurized these cavities with fluid for up to three months. In preliminary results, pressure dramatically improves tissue architecture, preserves biomimetic radial organization for longer than any existing protocol, and supports formation of single-rosette organoids over 1 mm in diameter. Inflated organoids show more uniform neural induction than controls, with a single continuous ventricular zone and uniform radial glial scaffold spanning the entire tissue, as in the embryonic cortex. Surprisingly, inflation also shows long-term effects on neural differentiation, demonstrating the importance of hydrostatic pressure for brain development and bioengineering. The inventors will characterize the resulting inflated organoids and uninflated controls using immunofluorescence, single-cell RNA sequencing, and spatial transcriptomics to understand how pressure can enhance organoid development from the molecular to tissue levels. This work introduces the most morphologically realistic cortical organoids to date, with potential applications for replacing animal models of hydrocephalus, congenital cortical malformations, and other neurodevelopmental disorders.

### Description:

The invention relates in some embodiments to a new technique for growing better-organized human brain organoids.

This work advances an alternative vision using bioengineering to better recapitulate cortical histogenesis, expanding the ability of cortical organoids to replace animals. In vivo, rosettes, like other radial glial scaffold anomalies, cause abnormal corticogenesis; thus, the inventors posit that assembling a cortex in vitro will require organoids with a stable single lumen. A recently-published protocol makes an important advance by allowing scalable generation of cortical organoids which initially form a single lumen; however, they subsequently develop multiple rosettes. Importantly, similar rosettes appear in chick, rodent, and human embryonic brains following experimental or spontaneous loss of eCSF pressure. Therefore, the inventors hypothesized that artificially inflating organoid lumens to mimic ventricle expansion will prolong biomimetic morphology, prevent hypoxia, and enhance cortical differentiation.

### Background and Preliminary Data

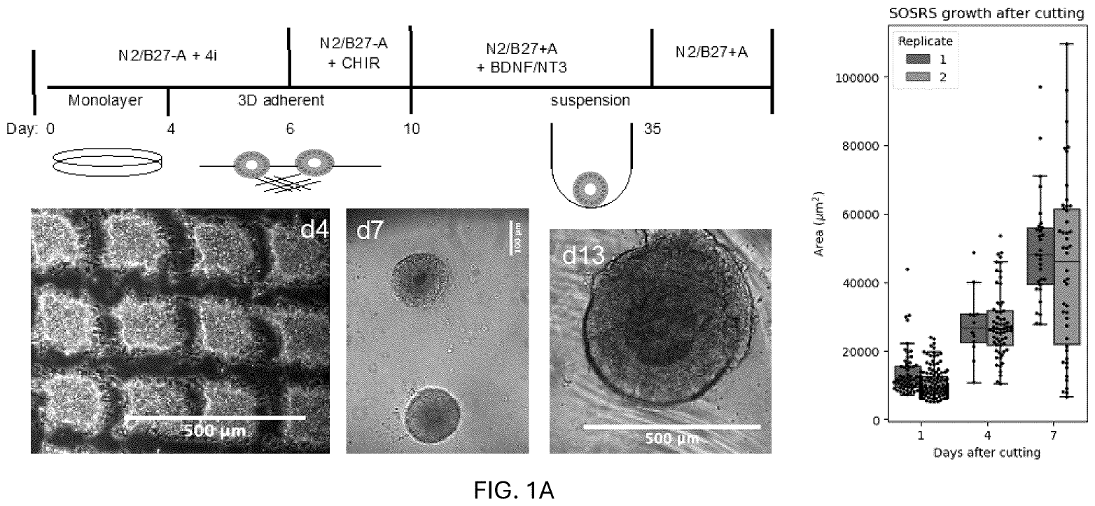

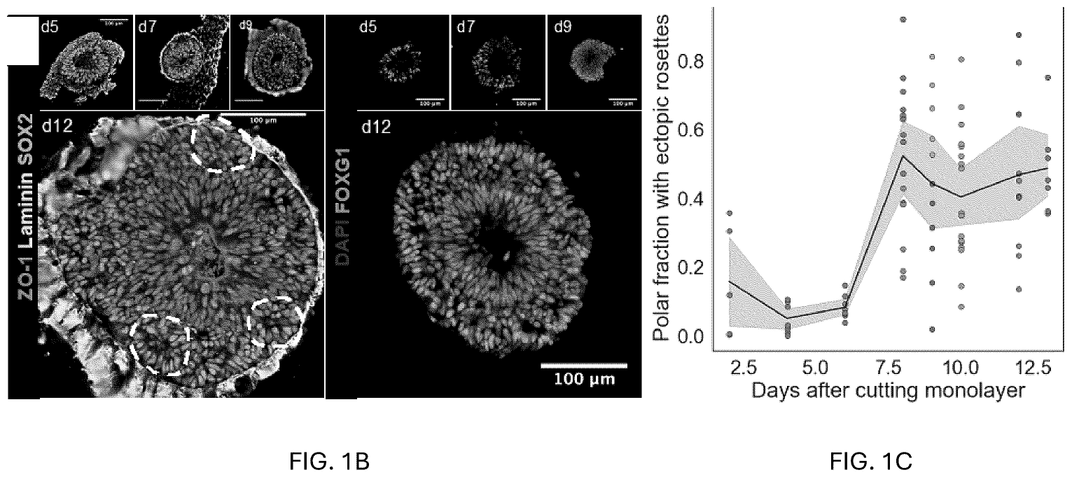

The inventors replicated single-rosette neuroepithelial organoids by inducing neural identity in an hiPSC monolayer and embedding cut pieces of monolayer in Geltrex with neurogenic media. By refining protocol timing, the inventors achieved high yields of healthy, uniform organoids that formed single rosettes two days after cutting (FIG. 1A). Immunofluorescence showed expression of neural stem cell marker SOX2, followed by embryonic forebrain marker FOXG1. Staining for ZO1, marking apical neuroepithelial surfaces, shows additional rosettes appearing within days, becoming dominant throughout the tissue within approximately eight days (FIGS. 1B-1C).

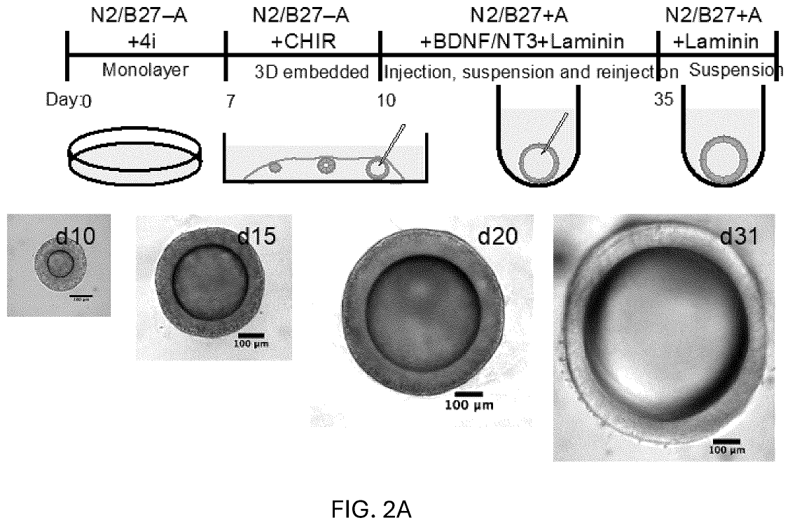

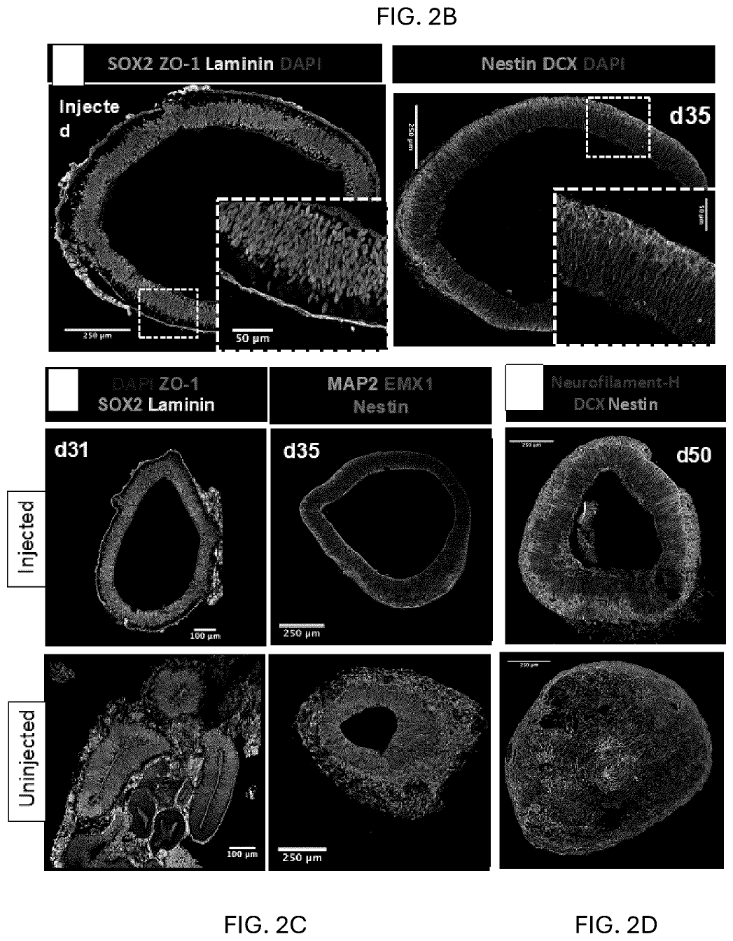

To test whether inflation stabilizes single-lumen morphology, the inventors microinjected fluid into organoid lumens, beginning two days after monolayer cutting and repeating every 3-5 days as needed, until harvesting for immunofluorescence between 1 and 3 months. Although some ruptured or produced unstructured outgrowths, others, termed inflated single-lumen organoids (iSLOs), maintained large single central lumens, while sham-injected controls lost organization (FIG. 2A). To assess whether physical or biochemical effects drove these effects, the inventors injected different batches with biomimetic hyaluronic acid solution or inert non-aqueous fluids; minimal differences were apparent, confirming that the observed effects are due primarily to hydrostatic pressure.

At 1 month, iSLOs and controls express SOX2 and ZO1, surrounded by laminin⁺ basement membrane. iSLOs showed symmetrical architecture with SOX2⁺ nuclei in a ventricular zone, a scaffold spanning the full tissue wall containing the radial glial marker nestin, and DCX⁺ newborn neurons migrating outwards (FIG. 2B). Notably, SOX2 signal was more uniform than in controls, where it varied between rosettes (FIG. 2C). Thus, iSLOs display uniform biomimetic architecture for at least three weeks longer than any currently published protocol, reaching diameters over 1 mm without superfluous rosettes or other anomalies. Although similar features appear in small areas within some conventional organoids, iSLOs are the first to sustain them throughout an entire organoid. This capability will enable future work to study early cortical radial migration in vitro and allow detection of subtle radial glial scaffold abnormalities that can have important clinical effects.

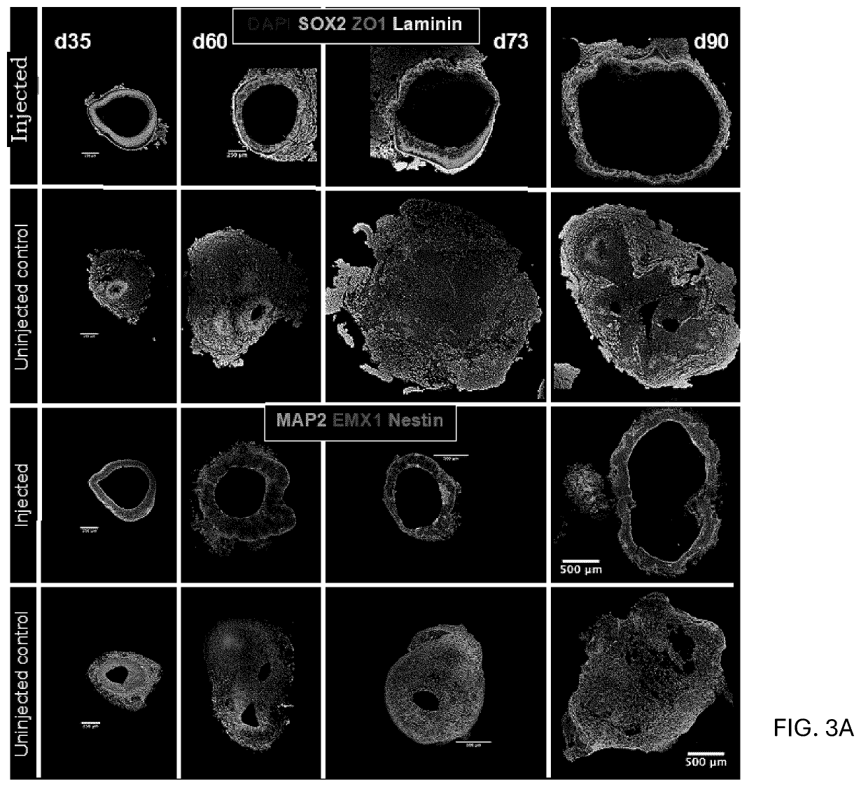

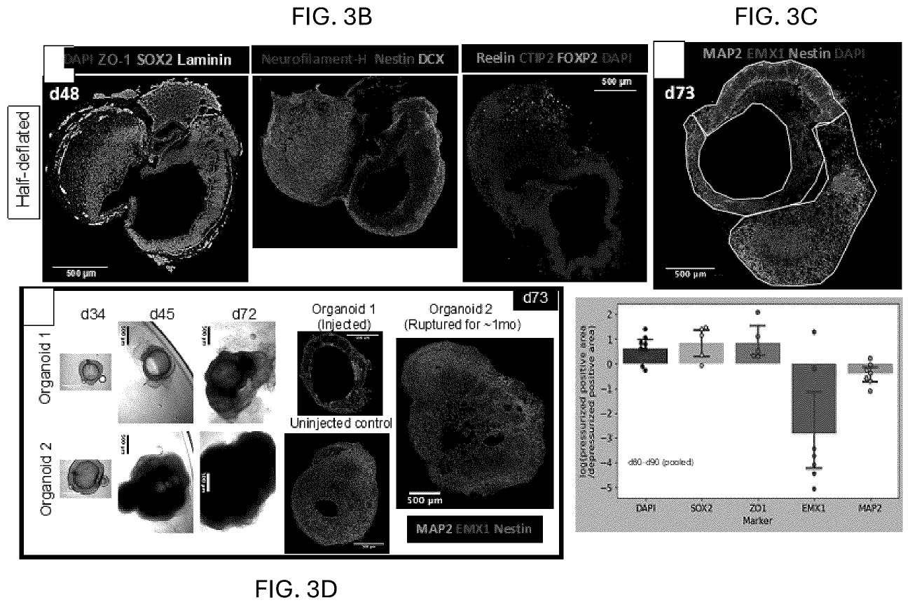

Although iSLO tissue architecture diverges from embryonic cortex at later stages, inflation continues to affect differentiation. By day 50, neurons overmigrate into an outer layer reminiscent of the cortical malformation cobblestone lissencephaly (FIG. 2D), and later form rosettes. Surprisingly, iSLO cells remain in a neuroepithelial stage marked by SOX2 and ZO1, unlike controls which progressively express the later stem cell markers EMX1 and nestin, and neuron marker MAP2 (FIG. 3A). Interestingly, within organoids where some tissue remained pressurized while another region deflated, depressurized cells underwent neurogenesis (FIG. 3B). Similarly, organoids that ruptured accidentally 30 days before staining show abundant neuronal differentiation (FIG. 3C). Together, these results confirm that hydrostatic pressure is a critical determinant of neurogenic differentiation, and that deflation strongly reverses pressure-induced inhibition to allow maturation.

These effects suggest that inflation and deflation represent powerful new tools for controlling the rate and timing of neurogenesis in cortical organoids, to more closely reflect embryonic development. Specifically, because the identity of progenitor offspring has been shown to be linked to developmental age in vivo, the inventors will test whether this protocol makes it possible to enhance the proportion of later-born neuroglial types by delaying neurogenesis.

### Experimental Aims

### Molecular Characterization of Biomimetically Organized In Vitro Cortical Progenitor Tissue

Neuronal specification and differentiation in brain organoids have been extensively studied by single-cell RNA sequencing (scRNA-seq). To situate iSLOs within this context, the inventors will analyze four iSLOs and two uninjected controls at 35 days by scRNA-seq (10× Genomics platform) and two iSLOs by spatial RNA sequencing (Slide-seq). The inventors will compare the resulting datasets with recent single-cell atlases of human embryonic cortex to quantify how inflation affects the recapitulation and progression of cellular identity and diversity.

Based on immunofluorescence results, the inventors expect iSLO neural differentiation to be more uniform and biomimetic than controls. Slide-seq will quantify gene expression as a function of apicobasal position, guiding future work on engineering organization in iSLOs. Following literature on mechanotransduction in embryonic neuroepithelia, the inventors will search for signatures of FAK-SMAD and Hippo pathway signaling in genes differentially expressed between iSLOs and controls.

### Describing the Long-Term Effects of Intraluminal Pressure on Organoid Differentiation

Intraventricular pressure falls in embryonic chick neural tube as neurogenesis begins, while induction of hydrocephalus in neonatal rats increases persistence of proliferative stem cells one month later. The results extend these findings in a human model, showing that intraventricular pressure strongly determines when neurogenesis can occur. The inventors aim to further characterize this effect and especially its reversibility, which could be important for basic developmental neurobiology and for bioengineering control over the timing of terminal differentiation in organoids, to better match human neurodevelopment.

The inventors will analyze organoids in three groups: inflated for 1 months or 3 months then intentionally deflated, and uninflated. At biweekly timepoints, the inventors will use dual EdU/BrdU incorporation to measure cell cycle length. The inventors will perform scRNA-seq on 2 organoids from each group at 35 days (described above), 2 and 4 months, for a total of 8 groups. Comparing to published single-cell analysis of human cortical tissue at different ages will answer key questions about how different periods of inflation affect neuroglial differentiation. The inventors will assess whether exposure to inflation affects the identity of neural stem cells at 1 and 2 months. The inventors will also compare postmitotic lineages at 3+1, 1+1, 0+1, 1+3, and 0+4 months (inflated+deflated). If progenitor maturation is “paused” during inflation, neurons after 1 month of non-inflation will be similar in identity and subtype composition, while if progenitors' competence “clock” continues to progress, those from 3+1 months will include a higher proportion of later-born cell types than 1+3 and 0+4, where depressurization allowed earlier terminal differentiation.

### Assessing Nutrient and Oxygen Stress in Inflated Organoids

Conventional organoids suffer from nutrient and oxygen stress in tissue ≥400 μm from the outer surface. Aggregating organoids around an inert scaffold can alleviate this problem, but at the cost of unnatural morphology. Because the iSLO tissue shell is <400 μm thick (FIG. 3), the inventors expect that inflation will offer similar benefits in a more biomimetic context. The inventors will search for genetic signatures of cell stress using the spatial transcriptomic and single-cell data above and validate results using molecular probes for hypoxia.

### Example 2

Work herein builds on prior published techniques, specifically those of Tidball, et al., Stem Cell Reports vol. 18:2498-2514 (Dec. 12, 2023), to adapt the protocols to produce the results described herein. In particular such modifications include:
More prolonged culture as a monolayer before cutting (7 days instead of 4) to advance differentiation further before forming a single rosette
Transferred cut monolayer squares to a layer of Geltrex® in a 3.5 cm culture dish instead of a deep 96-well plate to facilitate injection
The primary advance of the technique is the repeated manual injection of different fluids (silicone oil, hydroxyfluoroether, or a solution of hyaluronic acid in saline) to drive the inflation of the organoid and dramatically improve the quality and longevity of tissue architecture, as described in this detailed protocol. The advantage of hyaluronic acid is that it is an aqueous solution, so that in in the future, it can be combined with other bioactive water-soluble molecules to expose the ventricular zone of the organoid to ventricle-derived signals as have been reported in vivo but not the outer neurons.
Briefly, the inventors use a micromanipulator and glass capillary needle to inject organoid lumens in sterile conditions under a video microscope at day 10, before rosettes appear, and every 2-5 days thereafter, as needed to prevent organoid shells from becoming slack and folded.
Initial injection is performed while organoids are embedded in Geltrex®. Subsequently, organoids are isolated and moved to individual wells. Reinjections are performed by removing organoids from their individual wells with a wide-mouthed pipette tip, placing them on a low-attachment culture dish on the video microscope stage, and removing excess media so that the organoid is held in place by surface tension during reinjection, then returning organoids to their well.
The original protocol reports that without injection, organoids remain as single rosettes for less than 1 month, with a diameter of about 250 μm; the experiments have shown that additional rosettes appear within 12 days. With injection, organoids can retain a single lumen with no ectopic rosettes for 45 days, becoming over 1 mm in diameter with radial symmetry. FIG. 1C provides a chart showing the fraction of the periphery of fixed organoids that contains peripheral rosettes as a function of how many days have passed since organoids were formed from a cut monolayer.

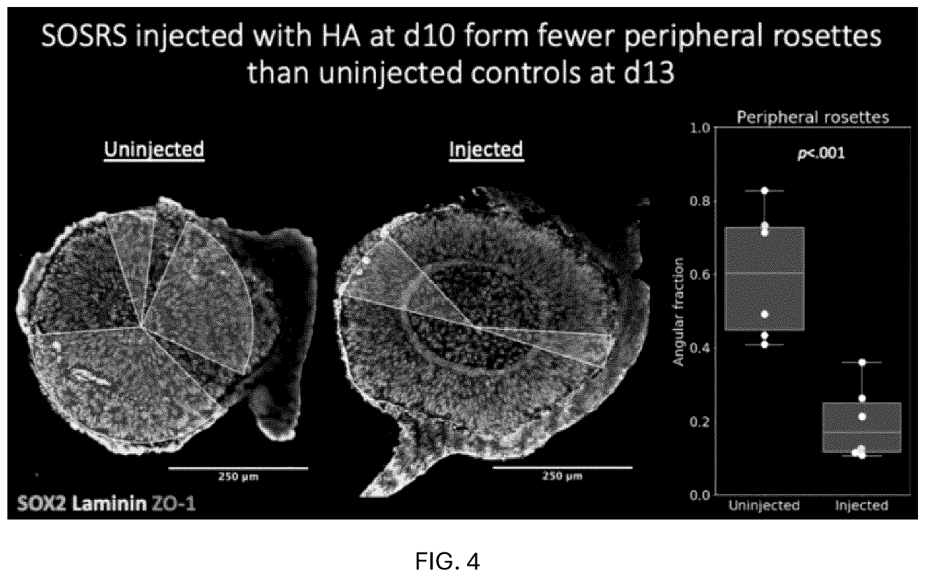

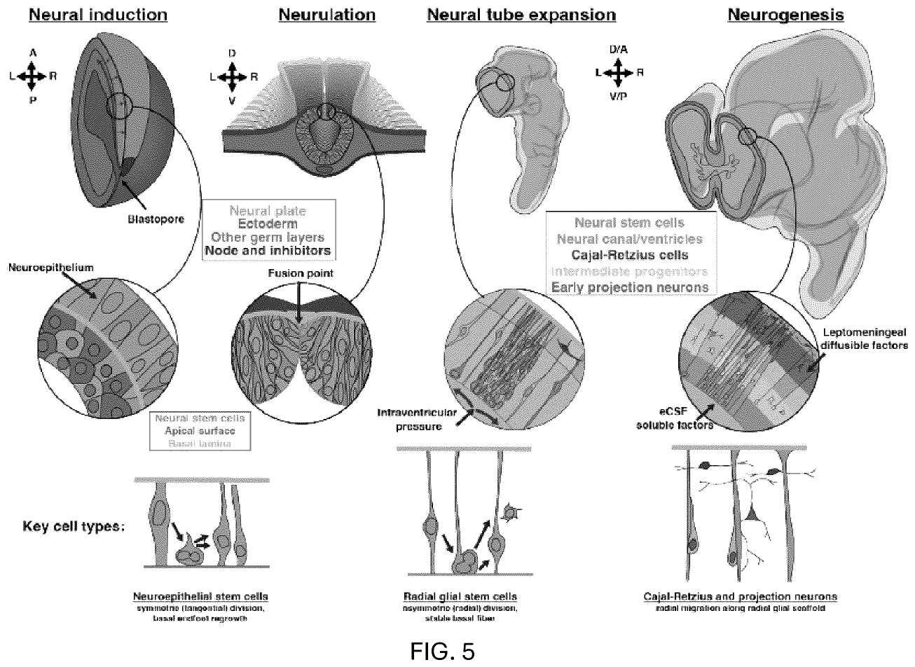

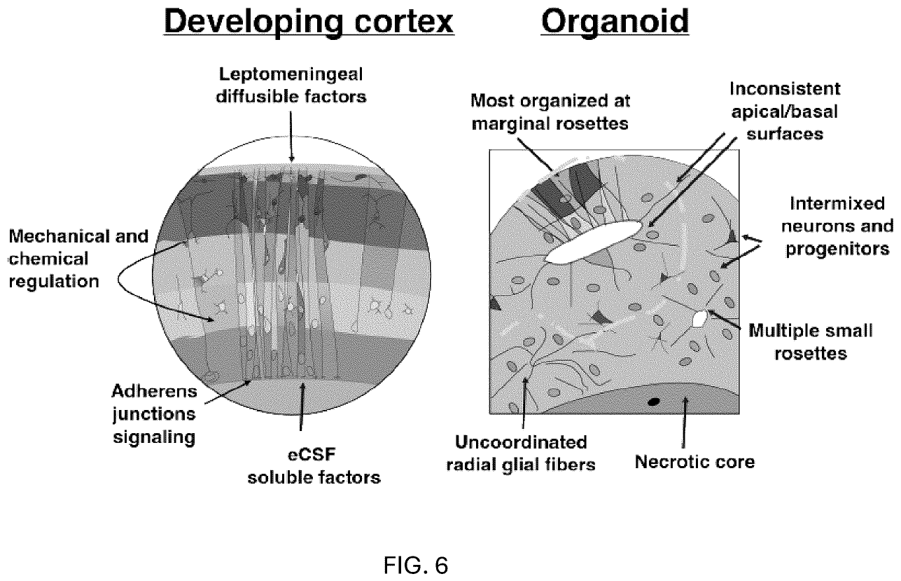

There is an example in FIG. 4 showing the results in two organoids. The average organoid has half its periphery filled with ectopic rosettes when fixed on day 8 after formation, although there continue to be very rare outliers with minimal rosette formation for a few days. In the uninjected organoid, many peripheral rosettes formed as in the previous batch. But the injected organoid has a much larger lumen and almost no rosettes. The inventors measured the angular fraction of the perimeter occupied by rosettes in each organoid, and it is evident that while the images the inventors chose are exaggerating the average effect a little, the difference was highly significant.

The original idea for this aim was to add dissolved basement membrane as a polarizing signal. These results show that a thick laminin+coating is not sufficient to prevent peripheral rosette formation, but it might be necessary, since areas where the outer laminin coating was compromised almost invariably become disorganized (cobblestone lissencephaly). Thus, laminin and swelling might work synergistically.

There are some cells inside the lumen here, which was sometimes but not usually seen in the last batch. Without wishing to be bound by theory, this may be because when monolayers are being cut and transferred to GelTrex®, the authors of the prior publication recommend dissociating gently for 60 seconds, but this seems longer than needed and sometimes causes some cells to delaminate and end up trapped inside the lumen after reorganization. Nonetheless, it demonstrates that nuclear density is decreased in 25 injected organoids, helping to visualize the swelling caused by HA injection.
Adding 0.5%-1% dissolved GelTrex® to culture medium after removing organoids from their embedded GelTrex®, to stabilize tissue architecture.

There are at least two additional benefits of inflation caused by the manual injection protocol described herein:
expression of neural stem cell markers is more even, likely due to more even exposure to the signaling factors in culture media and to unknown juxtacrine signaling effects by which progenitors influence their neighbors within the same rosette to share a similar identity.
Conventional organoids suffer from hypoxia and nutrient deficiencies in a necrotic core, due to the limited ability of oxygen and nutrients to penetrate into these non-vascularized tissues. Because inflated organoid cores consist of fluid instead of tissue, and the tissue shells are thinner than the oxygen diffusion limit, this problem is not observed in inflated organoids.

### Example 3: Inflation Controls the Timing of Neurogenic Onset in Single-Lumen Human Brain Organoids

### Introduction

### Brain Organoids as Models of Cortical Differentiation and Histogenesis.

Brain organoids are a reductive but powerful new model for exploring human cortical development, offering the opportunity to observe the emergence of all major forebrain-derived cell lineages in an experimentally-tractable system (Uzquiano, et al. 2022; Z. He, et al. 2024; Nasu et al. 2012). The accessibility of these 3D in vitro models facilitates direct interventions throughout the culture period, including physical manipulations such as slicing and electroporation (Qian et al. 2020; Kolodziejczyk et al. 2024), which could facilitate using organoids to investigate the biological effects of pressure. However, although organoids show high fidelity to human fetal brains at the molecular and cellular levels, they do not spontaneously inflate like the embryonic brain. Furthermore, organoids do not replicate the architecture of the endogenous brain: early organoids typically display multiple neuroepithelial rosettes, and as they mature, these rosettes break down into disorganized mixtures of neural cell types (Lancaster, et al. 2013; Velasco, et al. 2019; Chiaradia et al. 2023), with histology strikingly reminiscent of the depressurized embryonic brains described above. Recent work has sought to improve the recapitulation of cortical histogenesis in organoids by developing robust protocols for generating 3D neuroepithelial cysts and similar tissues that have a single lumen surrounded by biomimetic apical-in polarized neuroepithelial tissue, which can then be cultured to create cortical organoids (Pagliaro et al. 2025). However, this organization is short-lived: the latest timepoint at which biomimetic single-lumen organization was demonstrated to persist in organoids cultured using these approaches ranged from 11 (Abdel Fattah et al. 2021; Karzbrun, et al. 2021) to 21 (Xue et al. 2024; Jalilian and Shin 2023) or 22 (Tidball et al. 2023) days following initial differentiation. In light of the apparent promotion of rosette formation by depressurization in embryonic brains discussed above (Wilson 1972; Mittelbronn et al. 2008; Donkelaar et al. 2014), the inventors hypothesized that adding intraluminal pressure may be necessary to support normal histogenesis for longer timescales and at larger size in organoids.

### Goal: Investigate how Pressure Affects Developmental Timing.

The inventors sought to test the effects of biomimetic intraluminal pressure on tissue and cellular development in human brain organoids. The inventors hypothesized that intraventricular pressure would promote organized neuroepithelial growth, and also promote symmetric division of neuroepithelial stem cells at the expense of neurogenesis. To test this hypothesis, the inventors adapted a recently published model that enables scalable production of cortical organoids that initially have a single radially symmetric rosette (Tidball et al. 2023), making this protocol an ideal “single-rosette starting point” for further advances in facilitating biomimetic self-organization at later stages. Next, the inventors used microinjection to directly pressurize these single lumens with biosimilar or bioinert fluids.

### Summary of Results:

The inventors show that, regardless of the fluid used, lumen inflation dramatically improves organoid tissue architecture, extending the maintenance of single-lumen morphology from an average of two weeks to up to three months, while promoting continued neural stem cell division and inhibiting the birth of new neurons (neurogenesis). Experimental deflation of organoids reverses this effect, allowing rapid neuronal differentiation, which the inventors characterize using single-cell RNA sequencing. Transcriptomic analysis of experimentally inflated or deflated shows that these organoids match the cell types present in the original uninflated protocol, reveals improved segregation of neural progenitor and neuronal identity, and sheds light on the molecular pathways affected by inflation. These results demonstrate that intraluminal pressure is a potent regulator of the neurogenic switch in an in vitro model of human cortical development, with potential implications for understanding cortical evolution and neurodevelopmental disease.

### Methods

### Culture of Uninjected Self-Organizing Single-Rosette Cortical Organoids

For initial replication of self-organizing single-rosette cortical organoids in pilot experiments, the protocol from (Tidball et al. 2023) was followed as closely as possible. 3N media was prepared by combining 50 ml DMEM/F12 basal media, 50 ml Neurobasal media, 24 μl of a 10 mg/ml human insulin solution, 500 μl 100× non-essential amino acids, 500 μl 100× GlutaMax solution (equivalent to 200 mM glutamate), 90 μl β-mercaptoethanol 55 nM (Gibco 21985-023), 500 μl N2 supplement, 1 ml B-27 supplement with or without vitamin A depending on the stage of the protocol, and 1 ml penicillin/streptomycin solution. Aliquots of 1000×SB431542 (1000×, 10 mM in DMSO), 2000×DMH1 (4 mM in DMSO), 2000×XAV939 (4 mM in DMSO), 2000× cyclopamine (2 mM in sterile molecular biology-grade ethanol), 1000×CHIR99021 (3 mM in DMSO), 1000×NT3 (20 μg/ml in molecular biology-grade water with 0.1% BSA), and 5000×BDNF (100 μg/ml in 0.1% BSA) were stored at −80° C. for up to 3 months, and used within 3 days of being moved to 4° C. Cells were maintained in mTeSR+ media (StemCell technologies) and tested biweekly for mycoplasma contamination.

To collect these organoids for immunolabeling analysis of early tissue architecture, a wide-opening low-attachment P200 tip was used to gently break up ECM containing organoids, and this material was allowed to settle for 5 minutes in a 2 ml tube between collection, fixation with 4% paraformaldehyde in PBS solution, and three washes in PBS. Organoids were then embedded, sectioned, immunolabeled, and imaged as described below.

iSLCO Culture

Self-organizing single-rosette spheroids were prepared for injection using the procedure from (Tidball et al. 2023) with modifications. hiPSCs were cultured in mTeSR+ on 6 cm dishes pre-coated with 1% Geltrex to obtain healthy colonies with round edges and densely packed cells with large nucleoli. To seed wells, dishes with colonies just beginning to merge were used. Cells were passaged by incubating with Gentle Cell Dissociation Reagent for 4-5 min, followed by adding 2 ml stem cell media. Cells were detached using a cell lifter, dissociated to form clusters of 3-10 cells by trituration with a 5 ml pipette, and 300 μl of this solution was transferred to each of four wells of a 12-well plate that had been filled with room-temperature mTeSR+. iPSCs were fed daily by aspirating mTeSR+, washing gently and quickly with 1 ml PBS per well, and adding 2 ml fresh mTeSR+. The primary results reported here were generated using the male 11a human iPSC cell line from the Harvard Stem Cell Institute; authentication of this cell line is described in Quadrato et al. 2017. Karyotype analysis of this cell line was performed by WiCell Research Institute at the beginning of this project.

The date when cells reached 75% confluency was set as day 0 of the protocol. At this time, media was changed to 3N media without vitamin A (henceforth 3N without A) with 2 μM DMH1, 2 μM XAV939, and 10 μM SB431542 to begin differentiation. Media formulations and schedules throughout the protocol followed Tidball et al. 2023 except for a slight prolongation of the initial neural induction phase, as described here. Daily until monolayer cutting, 100% of the media was removed, wells were gently washed with warm DMEM/F12, and 2 ml fresh 3N without A with SB, XAV939, DMH1, and 1 μM cyclopamine (3N+4i) was added to each well. Monolayers that detached from the well and rolled up before day 8 were discarded. Monolayers that remained adherent, with at most slight rolling at dish edges, were cut and transferred to Geltrex on day 8. For each well to be transferred, seven 28 μl drops of Geltrex were placed on a 35 cm TC dish and a 200 μl pipette tip was used to spread them into a flat central surface covering most of the dish, and dishes were then incubated at 37° for exactly 20 min during monolayer cutting. 1.5 ml conditioned media was collected from each well and centrifuged for four minutes at 300 g to remove floating cells. The remaining media in each well was aspirated and washed very gently with 1 ml PBS to avoid detaching the monolayer. PBS was aspirated and replaced by 500 μl of conditioned media. Monolayers were cut twice using an EZpassage roller cutting tool to form a central square of evenly sized squares surrounded by a ring of strips and uncut monolayer. Conditioned media was aspirated and replaced with 500 μl Lonza L7 Passaging solution to each well. After 60 seconds, passaging solution was aspirated using a 2 ml aspirating tip along with all peripheral cells and monolayer strips not part of the central square. A P1000 tip was used to dislodge cell squares by spraying wells with 1 ml of reserved conditioned media, then 2 ml of fresh 3N+4i media. Using a 5 ml pipette, all 3 ml from a single well were gently mixed and added to a prepared Geltrex-coated 35 mm dish. Beginning on day 9, 1.5 ml media was then removed daily from each dish and replaced with 3N without A with 3 μM CHIR.

When SOSRS reached approximately 250 μm in diameter, which reliably occurred on day 11 (three days after transfer), brightfield microscopy was used to verify that almost all organoids had clearly visible lumens with crisp edges, that no organoids anywhere in the dish showed more than minimal cell death (appearing as a “halo” of rounded cells around individual organoids), and that plenty of organoids were spaced apart by approximately 1 mm to facilitate individual injection and transfer. Batches that did not pass these quality checks were discarded.

### Initial Injection

For injection of single-rosette organoids, custom glass needles and transfer pipettes were prepared. Nanoject glass capillaries (1.14 mm O.D, Drummond Scientific 3-000-203-G/X) were pulled in one step at 62.5° C. using a needle puller (Narishige, PC-10), starting approximately 1″ from the end and extending to a long point, at least 1″ long. Two needles were pulled from each capillary blank, one from each end. Needles were then ground for 25 seconds on a micropipette grinder (Narishige, EG-45) at a 45° angle to yield a sharp opening no more than 10 μm in O.D.

Needles were cleaned by spraying externally with 70% ethanol, attaching them using a small piece of flexible tubing to an aspirator in a sterile biosafety cabinet, and aspirating a small amount of sterile water. Needles were then filled with injection fluid. For silicon oil, needles were backfilled using a blunt Nanoject 30 Gauge Needle and 1 ml syringe and attached to the Nanoject micropipette directly. For hyaluronic acid, 20 μl of a 2 mg/ml solution of 50 kDa hyaluronic acid (HAworks HA-50k) or fluorescein hyaluronate (HAworks #HA-FITC-50k) in PBS with 0.1% dye solution for brightfield visualization was placed on a clean culture dish lid in the biosafety cabinet. A needle backfilled with silicon oil was loaded into the Nanoject micropipette and slowly (over the course of approximately 15 minutes) front-filled with HA solution, pausing as needed to allow equilibration of front-filled solution pressure as it slowly passed through the very small tip; excessively rapid front-filling will lead to cavitation of bubbles in the tip, making the needle unusable. For Fluo-Oil 7500 (hydroxyfluoroether; manufactured by Emulseo, purchased from Darwin Microfluidics #EU-FLUO-7500-20), 0.2 ml liquid was loaded into a 1 ml syringe and backfilled into a cleaned needle using a 1.5″×30 gauge straight blunt end needle (Fisher #8001114). Introduction of bubbles or contaminants into filled needles must be strictly avoided to allow smooth and successful delivery of injected fluid.

To inject organoids, a Nanoject II micropipette (Drummond), micromanipulator standard rubber vibration isolation pad were cleaned with 70% ethanol and set up in the biosafety cabinet along with a video stereoscope (EVOS XL core) such that the needle tip could be advanced to the center of the stereoscope focus area. A dish full of single rosette neuroepithelial cysts that passed the quality checks above was removed from the incubator and placed on the stereoscope stage. The researcher's non-dominant hand was then used to position individual organoids in the path of the needle, and the dominant hand was used to lower the tip into the center of the lumen, passing through the organoid shell at a point towards the side of the lumen. Once the needle tip was in position, the non-dominant hand or a foot pedal was used to inject fluid into the organoid, the tip was withdrawn, and the dish was moved to locate another organoid at the injection position. Initial injection volume was 2.3 nl, the minimum volume allowed by the micropipette. Injections were repeated for up to 30 minutes, after which dishes were returned to the incubator before another injection session if necessary. If gel or organoid material was observed to foul the needle tip, it was immediately replaced with a fresh tip to minimize disturbance to injected organoids.

Following injection of all symmetric, well-spaced organoids, 1.5 ml media in each dish was changed with 3N without A with CHIR and with 1% dissolved Geltrex and dishes with injected organoids were returned to the incubator, in order to allow spontaneous overnight failure of organoids damaged during initial injection.

Transfer into Individual Wells

24 hours after injection, organoids were transferred to individual wells for further culture. Organoids were inspected (using brightfield stereoscopy for silicon oil and Fluo-Oil injections or fluorescent stereoscopy for fluorescein hyaluronate injections) to ensure that at least 20% of injected organoids retained injected fluid. To transfer them, custom Pasteur pipettes were made. Borosilicate glass pipettes were melted and pulled using a Bunsen burner and forceps to form a 70° curvature close to the tip. This tip was then broken using forceps and ground using the needle grinder to form a flat opening with an outer diameter of 500 μm, measured using a stage micrometer slide and stereoscope. Finally, the grinder was used to bevel the edge of the opening from the outer to inner diameter at approximately 45° to form a sharpened edge. These pipettes were stored in a P1000 slide box and cleaned before each use by fitting them with a rubber bulb and repeatedly aspirating and ejecting first 70% ethanol, then distilled water, and finally DMEM/F12 basal media. To maintain cleanliness during use, pipettes were rested on a plastic pipette trough (VWR #10770-296) with a notch cut into the longest surface. To transfer organoids, a 96-well U-bottom tray (Sigma Aldrich, #CLS7007-24EA) was pre-filled with 100 μl of 3N without A with 1% Geltrex, 3 μM CHIR99021, BDNF (20 ng/ml), and NT3 (20 ng/mL) per well. A dish containing injected organoids was placed on the in-hood stereoscope stage with the lid removed. The custom pipette was held in the researcher's dominant hand, rested on the stereoscope stage for stability, while the bulb was held in the non-dominant hand. With the bulb lightly depressed and the tip guided by the stereoscope, the pipette was lowered over a single injected organoid that had retained its injected fluid; the bulb was then slowly released to aspirate the organoid along with approximately 50 μl of Geltrex and conditioned media. The contents were then transferred to a prepared 96-well tray well. After the tray was filled, wells were examined and any wells with multiple or zero organoids were corrected. Finally, an additional 100 μl of room-temperature 3N without A with 1% Geltrex was added to each well and trays were returned to the incubator. Uninjected control organoids were transferred from a separate dish kept in reserve and selected according to the same features as organoids to be injected, to minimize bias that could arise from inadvertently selecting organoids that failed to retain their injected fluid to use as controls.

### Extended Culture and Reinjection

Organoids were fed by exchanging 100 μl of media with fresh 3N with vitamin A with 1% Geltrex, beginning on the second day after transfer to wells (day 14) until day 20. Organoids were subsequently fed daily to minimize metabolic stress as they grew. On day 35, surviving inflated organoids and uninflated controls were transferred to individual wells in 1 ml of 3N with A with 1% Geltrex but without BDNF and NT3, following the Tidball et al. 2023 protocol.

Each organoid was imaged every other day using a brightfield stereoscope, and the shell thickness was measured using Leica LAS X imaging software. Organoids were reinjected when their shell thickness exceeded a critical value. For most experiments, this value was set at 100 μm until day 30 and 200 μm until day 60, and thereafter organoids were reinjected when the primary shell tissue appeared slack rather than turgid. For the experimental deflation experiments described herein, this threshold was set according to a more conservative schedule inspired by the thickness of the growing human embryonic cortical anlage (Bayer and Altman 2006; Ze ̆cevic 1993). In these experiments, the threshold used began at 100 μm on day 10 (initial injection day) and was raised to 200 μm on day 16, 300 μm on day 21, and 375 μm at day 27 and beyond.

To reinject organoids, a wide-opening P200 low-attachment tip (Rainin) was used to transfer organoids from a 96-well onto the center of a clean culture dish lid on the in-hood stereoscope stage, then to aspirate all but a thin film of media to hold the organoid in place using surface tension. The pipette was set aside, resting on a plastic reservoir, and a glass needle was then used to reinject organoids as before. Organoids were injected until they appeared taut and near-spherical, and the shell was stretched to below the appropriate thickness limit; excessive injection can cause organoid rupture. The manual pipette was then used to resuspend the organoid in its original culture media and return it to its well. The culture dish lid was replaced whenever gel or tissue residue was observed.

Organoids were scored as remaining in the inflated single-lumen cortical organoid category if all of the following criteria were met in brightfield images: they had a large, visible central lumen with no other macroscopically apparent lumens, they did not rupture and release their injected fluid (a frequent occurrence during handling and reinjection), and the estimated combined size of any disorganized outgrowths, added together, did not exceed the size of the central symmetric shell.

### Cryosectioning and Immunolabeling

Immunohistochemistry was performed as described (Velasco et al. 2019) with slight modifications. Samples were fixed in 4% paraformaldehyde (Electron Microscopy Services), cryoprotected in 30% sucrose solution in PBS, embedded in a warmed solution of 10% bovine gelatin and 7.5% sucrose in PBS, frozen using a slurry of ethanol in dry ice, and cryosectioned to a thickness of 14 μm thickness. To prevent damage to the soft gelatin blocks, cryosectioning was performed at unusually low temperatures ranging from −31 to −26° C. Sections were washed with 0.1% Tween-20 (Sigma) in phosphate buffered saline (PBS) (Gibco), blocked for 1 h at room temperature with 7.5% donkey serum (Sigma) and 0.3% Triton X-100 (Sigma) in PBS, then incubated overnight at 4° C. with primary antibodies diluted in 2.5% donkey serum and 0.1% Triton X-100 in PBS.

### Multiplexed Single-Cell RNA Sequencing

For the single-cell transcriptomic experiment, inflated organoids at day 42 were randomly assigned to ongoing reinjection or to be deflated by placing them on a tissue culture dish and using a P1000 tip to remove all surrounding media, pressing the organoid flat by surface tension and leading to organoid rupture. Organoids were then returned to culture with or without reinjection according to the threshold schedule described above. On day 49, individual brain organoids were dissociated into a single-cell suspension using the Worthington Papain Dissociation System kit (Worthington Biochemical), following a detailed protocol for organoid dissociation (Velasco et al. 2019). Dissociated cells were resuspended in warm ice-cold PBS containing 0.04% BSA (Sigma, PN-B8667), counted them with a Countess II automatic cytometer (Thermo Fisher Scientific), and adjusted to a final concentration of 1,300 cells/ml for a target recovery of 40,000 cells. Before library construction, samples derived from individual organoids were incubated with one of four Cell

Multiplexing Oligos to allow samples to be sequenced together and computationally separated after sequencing. Cells were loaded onto a Chromium Single Cell 30 Chip (10× Genomics, PN-120236), and processed through the Chromium Controller to generate single-cell GEM (Gel Beads in Emulsion) libraries. scRNA-seq libraries were prepared with the Chromium Single Cell 30 Library and Gel Bead Kit v3.1 (10× Genomics, PN-1000121). Libraries were pooled with libraries from other from different samples based on molar concentrations and sequenced on a NovaSeq 6000 instrument (Illumina).

scRNAseq Processing and Annotation

CellRanger Multi was used to align scRNA-seq reads to the GRCh38 human reference genome and to demultiplex tagged cells from individual organoids. CMO assignments from this pipeline were used with default settings, and unassigned and multiplet cells were excluded from analysis.

Data was analyzed using the Seurat R package v5.0.3 (Hao et al. 2021) using R v4.3.3. Cells with between 500 and 20,000 reads, a minimum of 200 unique features, and less than 15% mitochondrial genes were kept for downstream analysis. For analysis of differentially expressed genes and gene modules, expression data from the deflated and reinjected channels were separately scaled and normalized using Seurat's SCTransform function. Cell cycling differences between G2/M and S phase were regressed out in separate objects along with percentage of ribosomal and mitochondrial genes. Seurat objects representing deflated and reinjected conditions were clustered and annotated separately to preserve biological differences in cell type composition. Objects were then merged and integrated for downstream analysis, beginning with a second SCTransform processing step to regress ribosomal and mitochondrial genes, cell cycle differences, and the number of RNA reads and features in each cell. FindConservedMarkers was used to identify genes that marked the clusters annotated as cortical progenitors across both conditions; these included many canonical neural stem cell markers including SOX2, HES1, GLI3, SHROOM3, and FOXP1. FindMarkers was then used to identify genes upregulated or downregulated between these cells in each injection condition.

To identify common pathways or biological processes involved in these genes, enrichGO was used to identify GO terms and Seurat's AddModuleScore function was used to assign module scores relative to background expression of randomly selected non-module genes. To identify candidate gene sets in an unbiased manner, the inventors used the Hallmark and Canonical Pathway gene sets from the Human Molecular Signatures Database (MSigDB) (Subramanian et al. 2005; Liberzon et al. 2011; Liberzon et al. 2015). In addition, the inventors tested specific gene sets to test for expression of targets of specific mechanotransduction pathways, using the ChIP Enrichment Analysis (ChEA) dataset that compiles genes identified as epigenetically regulated by specific signals on the basis of low- and high-throughput functional chromatin expression profiling studies (Lachmann et al. 2010). For a final list of 30 gene sets, the inventors used Seurat's AddModuleScores function to calculate the average expression of each gene set in each cell, relative to the expression of control genes selected at random from each cell (Tirosh et al. 2016). Using these scores, the inventors calculated the effect size and significance of the change in module expression in cells of each identity cluster between deflated and inflated organoids and applied a Bonferroni correction to the significance threshold to account for the total number of tests performed.

### Nucleus Segmentation and Marker Analysis

To automatically quantify the proportion of nuclei positive and negative for immunolabelled nuclear markers across time, 20× images of sectioned organoids were collected using a Zeiss ApoTome system. A z-stack of 3-7 images spaced 1 μm apart was collected for each image, and was deconvolved, stitched, orthogonally projected, and exported in uncompressed tiff format using ZEN Pro software. At least 3 sections were collected from each organoid to be analyzed. A custom Python script was used to read in images and perform nuclear seg-mentation of the DAPI channel using CellPose (Stringer et al. 2020) with the pretrained “nuclei” model. Images were loaded and processed using an approach reported by Haofei Ge (available at github.com/MouseLand/cellpose/issues/1026), which was necessary to allow cluster-enabled script performance to equal that observed in the slower GUI. Mean intensity of the immunofluorescence signal in each channel corresponding to a nuclear marker was analyzed using the ndimage function from the SciPy package. Means were automatically thresholded as positive or negative by computing the Otsu threshold from scikit-image for the nuclear mean measurements from each section individually and from all nuclei in that channel collected on the same slide, then designating nuclei as positive for that marker if their mean value was greater than the average of these thresholds. For significance testing, a hierarchical mixed linear effects model from the statsmodels package was used to model nuclear marker expression as an additive linear function of age and injection group (separately comparing uninjected vs. HFE-injected and uninjected vs. HA-injected), with technical variance between individual sections of the same organoids as an additional level of variance.

### Results

### Single-Rosette Structure is Rapidly Lost in Uninflated Organoids

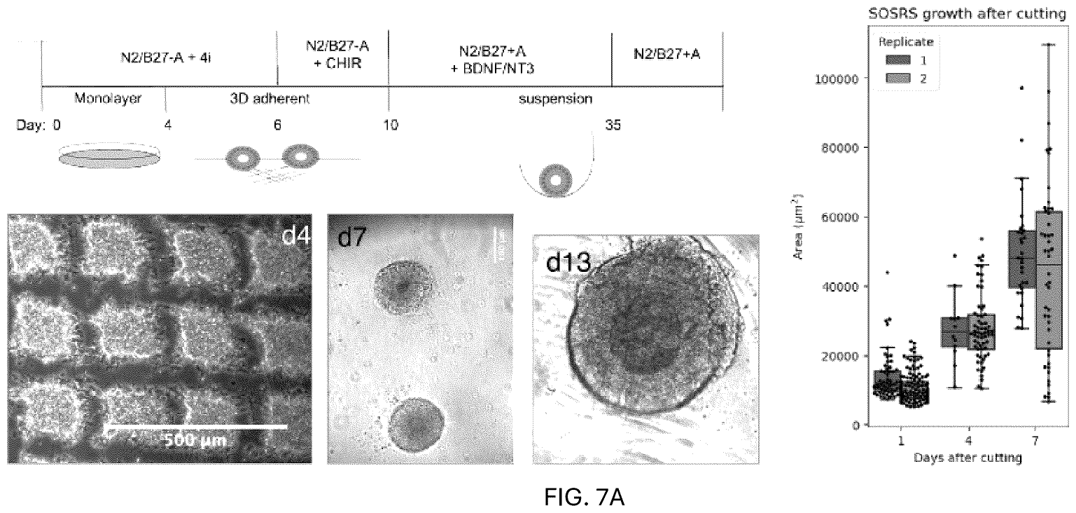

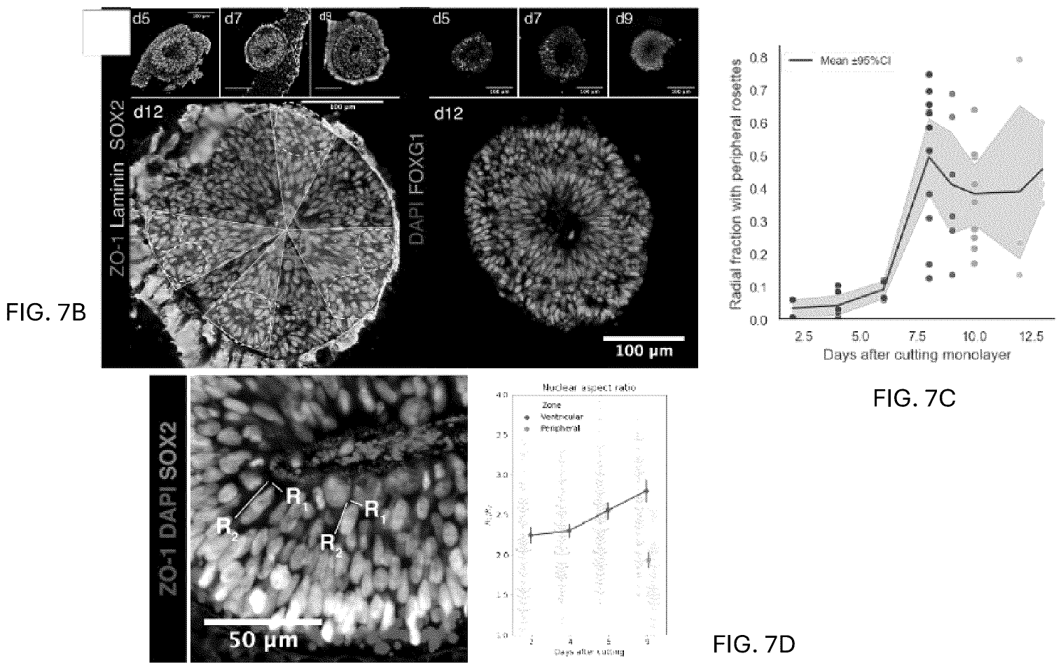

To study the emergence of tissue architecture in human brain organoids, the inventors adapted a recently published protocol for reproducible, accessible, and scalable formation of unilocular neuroepithelial cysts from human pluripotent stem cells (Tidball et al. 2023). In this protocol, hPSCs undergo neural induction as a monolayer before being cut into uniform pieces and transferred to a bed of Geltrex for reorganization. This approach yields single-lumen organoids with over 84% efficiency at two days after monolayer cutting. However, this organization was reported to be less homogenous at one month, and progressively lost at later timepoints (Tidball et al. 2023). The inventors replicated this protocol and confirmed that cut pieces formed single rosettes with high efficiency (FIG. 7A). To characterize the stability of organoid architecture in this protocol in greater detail, the inventors collected organoids 2 to 13 days after transfer to Geltrex for analysis by immunohistochemistry (IHC). Each rosette showed a central surface marked by the apical junction marker ZO-1, with the radial glial intermediate filament nestin organized in a radial pattern around this surface. Nuclear expression of neural progenitor marker SOX2 and forebrain progenitor marker FOXG1 indicated that these rosettes represented early forebrain progenitors. The inventors observed that nuclei surrounding the central lumen became increasingly densely packed and elongated in the days following organoid formation, followed by delamination of SOX2-positive neural stem cells from the central lumen and reorganization to form additional rosettes around the periphery of the organoid (FIG. 7B). The inventors measured the radial fraction of each section in which the central neuroepithelial architecture was interrupted by these ectopic rosettes, finding a large increase in this mean value between days 6 and 8 on Geltrex (FIG. 7C), indicating that this protocol does not consistently recapitulate stages of cortical histogenesis after initial apicobasal polarization of the neuroepithelium after the first days after transfer.

While organoids grew several times larger in diameter across this time period, most lumens showed minimal expansion. Notably, in the embryonic mouse ventricular zone, actomyosin-dependent apical constriction creates inward tension, and experimentally induced apical overcrowding of nuclei has been shown to cause neural stem cells to delaminate from the ventricular surface, resulting in heterotopic clusters of neural progenitors undergoing mitosis basal to the ventricular zone (Okamoto et al. 2013; Chenn and Walsh 2002; Lavado et al. 2018; Saito et al. 2018; Miyata 2023), similar to those observed here. Consistent with this possibility, microscopic nuclear morphometry in uninjected single-rosette organoids indicated that nuclei were increasingly compressed, with a mean aspect ratio growing to 2.6 in the ventricular zone; after peripheral rosettes appear, nuclei in these rosettes became much less elongated, with a mean aspect ratio below 2 suggesting that their delamination from the apical surface relieved their physical crowding (FIG. 7D). Therefore, these results provided further motivation for testing the effects of intraluminal pressure on the preservation of neuroepithelial tissue architecture.

### Injection of Single-Lumen Cortical Organoids Prolongs Single-Lumen Organization

The inventors modified the protocol developed by Tidball, Niu and colleagues (Tidball et al. 2023) to induce pressure and expansion in the lumens of single-rosette cortical organoids for up to three months. First, the inventors extended the initial 2D patterning period from four to seven days, seeding at a slightly lower density to prevent “rolling-up” before this timepoint. It was reasoned that this longer interval more closely matched the duration of neural induction between gastrulation and neurulation in humans (Stiles and Jernigan 2010) and could yield slightly more mature neuroepithelial stem cells with lower proliferative capacity and structural plasticity, thereby improving rosette stability.

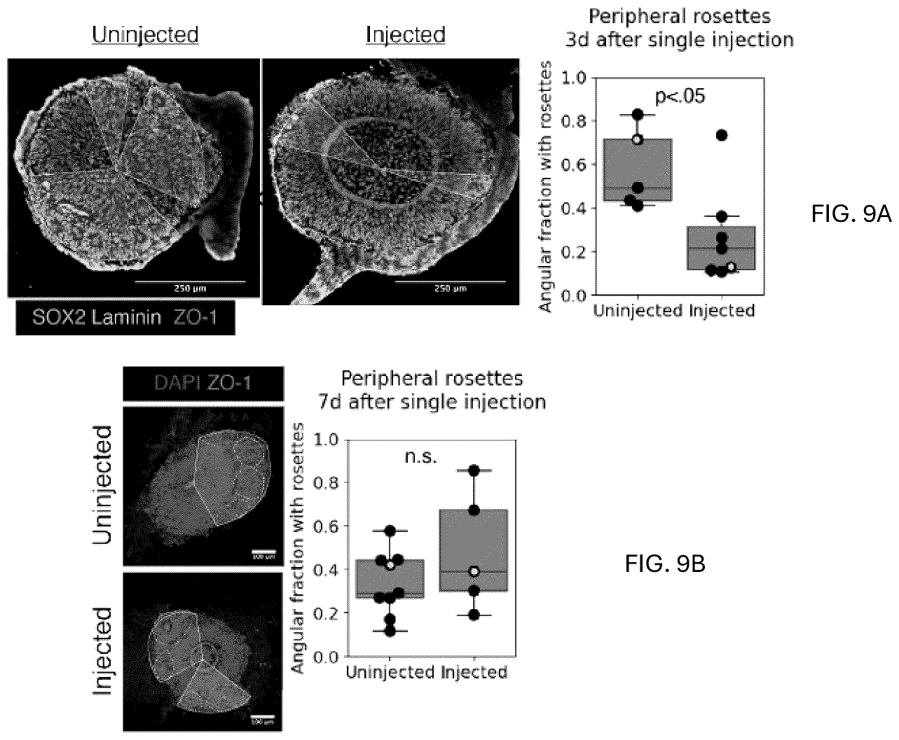

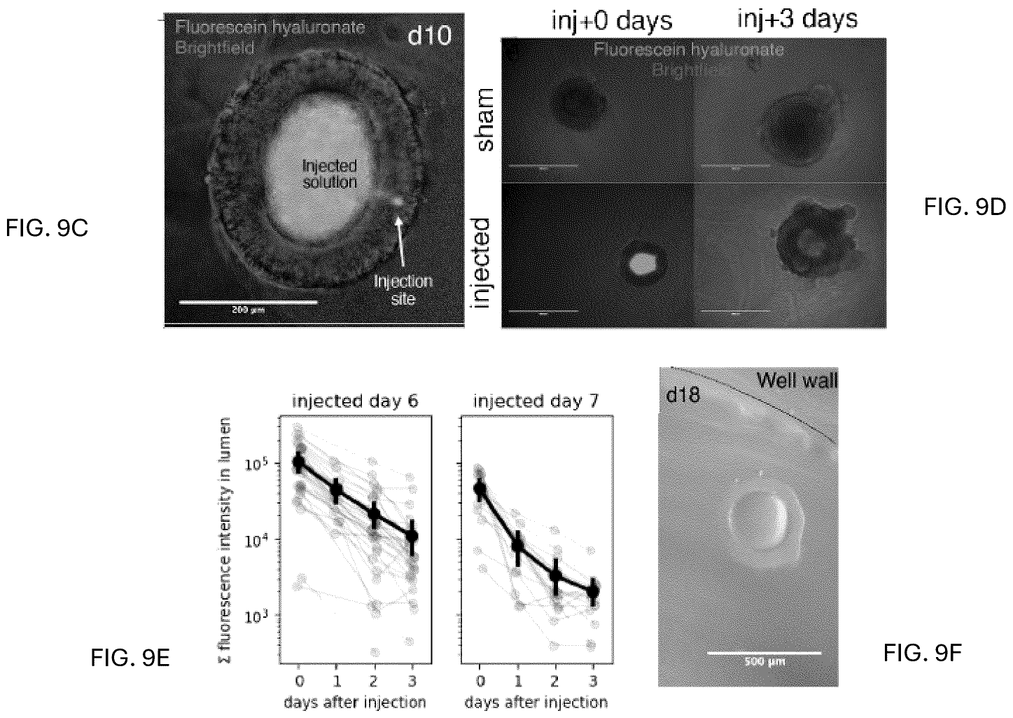

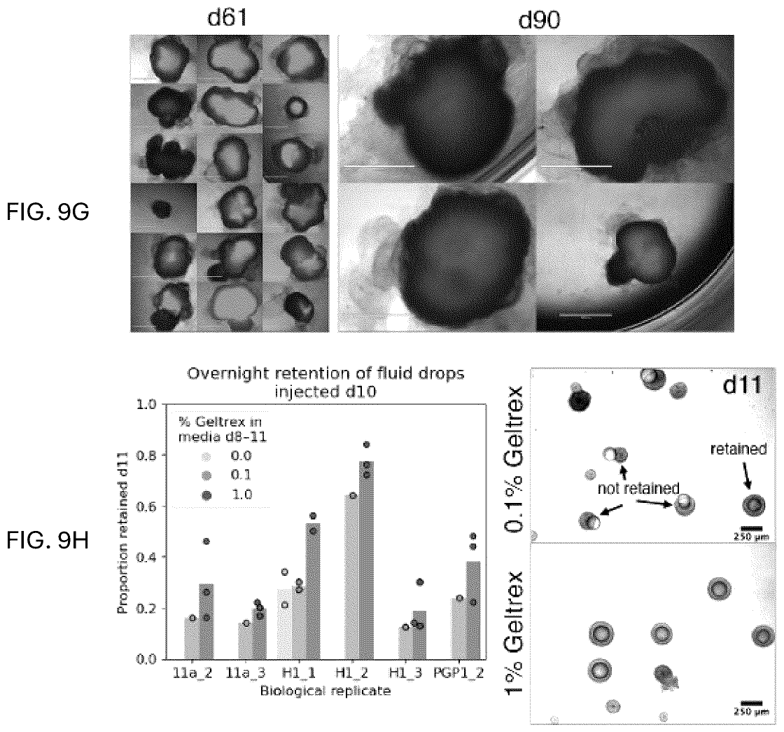

Next, the inventors used microinjection to individually pressurize organoid lumens. Three days after monolayers were cut and replated on a thick Geltrex bed, when pieces showed uniform spherical morphology (FIG. 9H), the inventors used a micropipette (Nanoject II, Drummond Scientific) and pulled glass needle to manually inject organoid lumens with fluid to imitate the expansion of the embryonic neural tube under positive pressure. In pilot experiments, the inventors first tried one-time injection of hyaluronic acid (HA) solution into organoid lumens, mimicking the endogenous secretion of HA along with other osmotically active macromolecules in the early embryonic neural canal (Alonso et al. 1998). This treatment reduced rosette formation after 3 days (FIG. 9A), but this effect was not observed at 7 days (FIG. 9B). Injection of fluorescein hyaluronate solution to visualize retention (FIG. 9C) confirmed that the fluorescent signal (proportional to retained fluid) in each organoid decayed rapidly, with a half-life of approximately 1 day (FIGS. 9D-9E). Therefore, the inventors adopted a strategy of repeated injection. Organoids were examined every other day using a stereoscope, and reinjected whenever the shell wall exceeded a predetermined maximum thickness informed by the thickness of human embryonic neuroepithelium. This threshold began at 100 μm and was raised over time to match the thickness of the developing human pallium cerebral wall. To control for the possible biochemical effects of specific injected fluids, this strategy was repeated using different injecta, including a simple biomimetic eCSF substitute consisting of hyaluronic acid and dye in saline solution, silicon oil, and Fluo-Oil 7500 (HFE; also known as hydroxyfluoroether-7500, HFE7500, and Novec™ 7500 Emulseo), an inert, denser-than-water fluid impermeable to most organic molecules that has been used to encapsulate living cells in microfluidic applications and in which most organic chemicals are insoluble (Baret 2012; Prastowo et al. 2016; Xia et al. 2016). Silicon oil allowed easy visualization of inflation due to its contrasting index of refraction, but led to flotation as reinjection gradually increased the volume fraction of organoid lumens (FIG. 9F). In contrast, HFE- and HA-injected organoids were denser than the culture medium; organoids injected with a solution of fluorescent HA (fluorescein hyaluronate) also allowed the use of epifluorescence stereoscopy to visualize the inflated core through the shell, which grew opaque at later ages. Therefore, HA and HFE were chosen for longer-term experiments. As a final minor change to the protocol, addition of 1% Geltrex dissolved in culture media between days 8 and 11 was found to significantly reduce organoid rupture and loss of injected fluid drops over the first 24 h of injection (p<0.005) (FIG. 9H); 0.1% Geltrex was also added throughout the remaining culture period, consistent with evidence that an externally deposited layer of ECM facilitates neuroepithelial polarization in organoids (Martins-Costa et al. 2023).

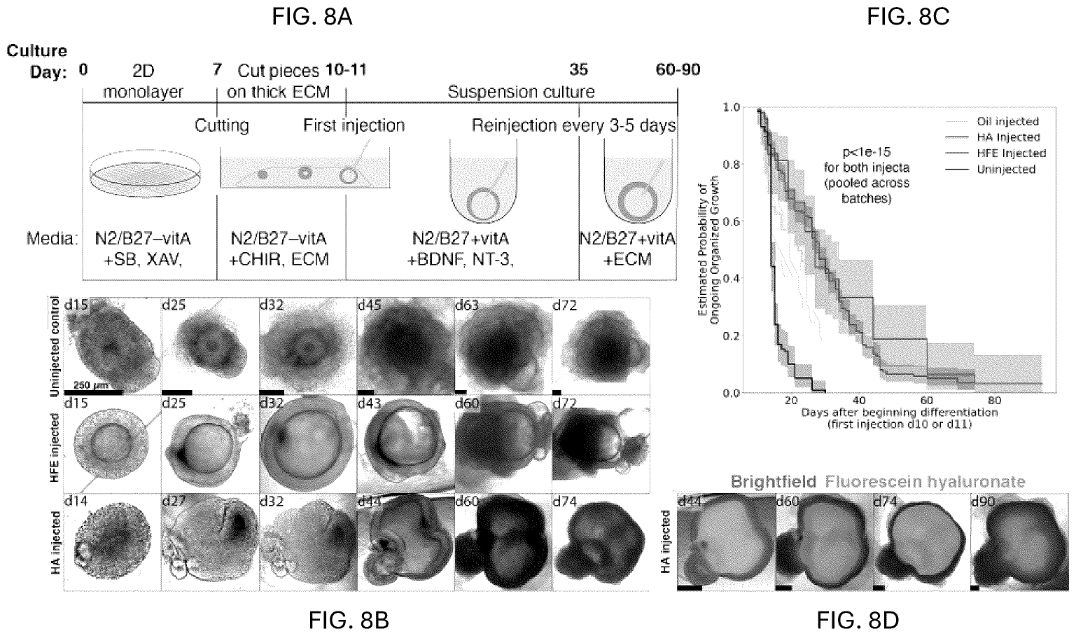

Monitoring of organoids using brightfield microscopy showed that, although many ruptured during manual handling and reinjection or eventually developed ectopic outgrowths, many injected organoids retained single-lumen organization as they grew (FIG. 8B) with 43% ( 308/710) remaining organized until at least 30 days after differentiation and 2% ( 14/585) until at least 60 days (FIG. 8C). In contrast, no uninjected controls out of 125 analyzed retained unilocular gross morphology beyond day 30. These constructs, which were termed inflated single-lumen cortical organoids (iSLCOs), could be reinjected for up to 3 months, the latest timepoint collected (FIG. 8D)

Inflation Allows Growth of Thick Neuroepithelia with Biomimetic Polarized Architecture

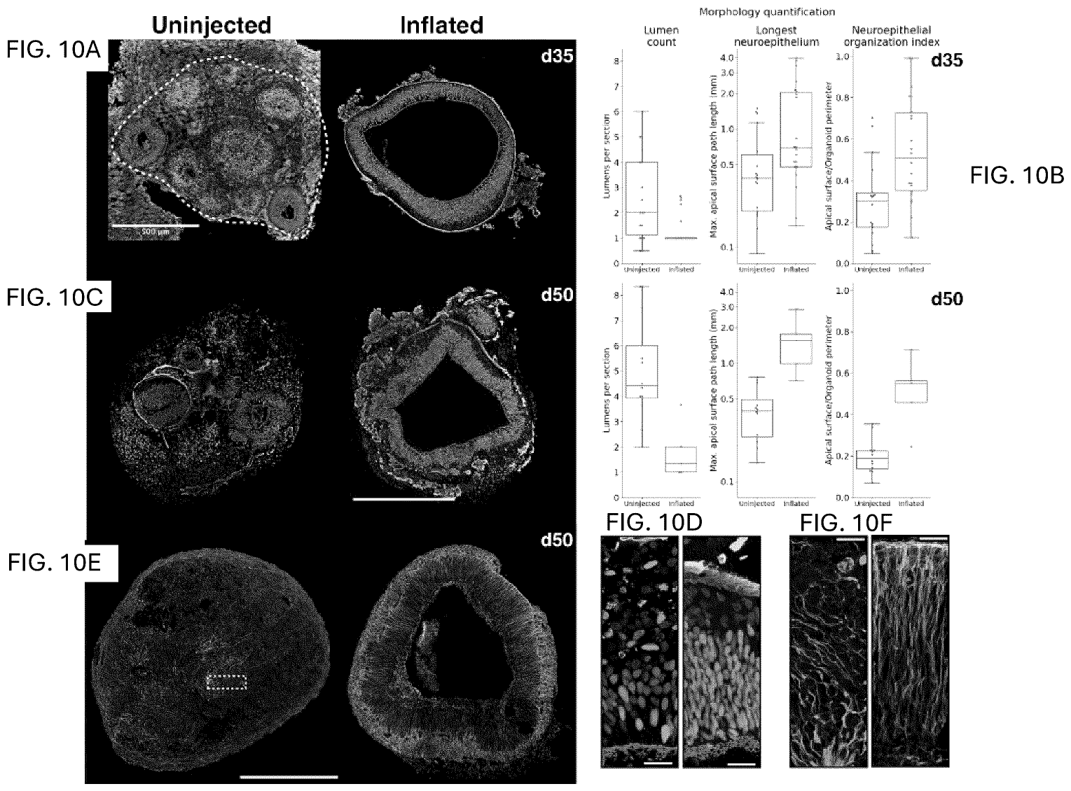

Immunolabeling of successfully re-injected organoids at day 35 of culture (24 days after first injection) showed extensive to total biomimetic pseudostratified neuroepithelial organization, with a single ZO-1-positive surface lining a large central lumen and a single layer of basement membrane at the outer surface of the organoid (FIG. 10A). To quantify these effects, n=16 iSLCOs and n=14 control organoids were sectioned and immunolabeled at 35 days of culture, and the perimeter of each organoid was manually traced along with the path length of each ZO1-positive apical surface, for at least 3 sections from each organoid. On a per-organoid basis, iSLCOs had fewer lumens per section (1.3 vs 2.6; p=0.01) and longer maximum stretches of apical surface (1.5 vs. 0.5; p=0.01). To characterize the overall neuroepithelial organization of the organoids, the neuroepithelial index was defined as the ratio of the sum length of apical surface in each section to the basal perimeter of the section. For sections occupied by uniformly polarized epithelial sheets, this index will be slightly less than one, whereas in those containing rosettes with small foci and more extra-rosette tissue, it will be lower. The inventors confirmed that this index was higher in iSLCOs than in control organoids (p<0.005). In iSLCOs, the thickness of the ventricular zone was approximately 80±20 μm, while an early marginal zone containing SOX2-negative nuclei between the VZ and basement membrane was approximately 30±10 μm thick; these measurements are close to those reported in human embryos at 35-42 days post conception, namely 100-140 μm in the ventricular zone and 25-40 μm in marginal zones (Ze ̆cevic 1993). These results show qualitatively and quantitatively that exogenous pressurization of single-rosette brain organoid lumens enables significant improvements in the survival and extent of biomimetic neuroepithelial organization after one month.

As they continue to mature, iSLCOs continue to build on this improved apicobasal surface polarization and display robust improvements in the formation and maintenance of the radial glial scaffold. At day 50, comparing n=5 iSLCOs with n=12 uninjected controls, iSLCOs again had significantly fewer lumens per section (p<0.01), longer stretches of neuroepithelial apical surface (p<0.001), and a higher neuroepithelial index (p<0.001) (FIG. 10D). Qualitatively, control organoids showed increasingly diffuse and uneven expression of SOX2 as their multiple rosettes began to intermix with non-stem cells (FIG. 10D). In contrast, iSLCOs had relatively dense, well-organized ventricular zones with uniform expression of SOX2 restricted to these zones and a thin layer of SOX2-negative nuclei in a marginal zone, comparable to the human neuroepithelium between Carnegie stages 16 and 22 (42-54 dpc; Eze et al. 2021). The thickness of the ventricular zone was approximately 130±40 μm, matching the range reported for the human cerebral VZ at 49-56 days post conception (90-170 μm; Ze ̆cevic 1993). The putative marginal, intermediate, and preplate zones were not easily resolved, but the total distance from the clearly defined SOX2-positive ventricular zone to the surface of iSLCOs ranged from 60-230 μm, again consistent with 7-week human embryonic telencephalic cortex, which has intermediate zone of 65-225 μm and marginal zone of 15-40 μm (Ze ̆cevic 1993; Bayer and Altman 2008).

When multiple rosettes were present in a single organoid, expression of SOX2 often varied appreciably between rosettes, consistent with more detailed recent analyses (Chiaradia et al. 2023; Singh 2024). Qualitatively, SOX2 expression around the single central lumen of single-rosette organoids appeared relatively uniform. Notably, ZO-1 continued to be strongly present at apical surfaces, and at high resolution could be observed to form a honeycomb structure outlining every apical cortex (FIG. 10D). This could indicate a molecular identity comparable to the early stages of neural expansion, at which ZO-1 immunoreactivity is abundant in human neuroepithelium, rather than the end of this range at which it becomes absent (Eze et al. 2021).

Confirming the degradation of rosette organization in control rosettes at day 50, all control organoids showed poor organization of nestin-positive radial glial processes, which formed disordered networks of fibers centered around rosette foci. In contrast, iSLCOs showed a robust, dense array of nestin-positive fibers oriented parallel to each other and perpendicular to the lumen surface, resembling the embryonic radial glial scaffold (FIG. 10E). Cells positive for the newborn neuroblast marker DCX were abundant throughout control organoids, while in iSLCOs DCX-positive cells could be observed extending processes along the radial glial scaffold and accumulating at the basal surface (FIG. 10F). Neurofilament-H, an intermediate filament marking more mature neurons, was sparse at this timepoint but concentrated at the periphery of the organoids. Although ectopic rosettes were occasionally present at the organoid periphery, most of the tissue comprised well-organized cortical anlage. However, the basal membrane was often discontinuous, and newborn neuroblasts that appeared to migrate basally from the ventricular zone increasingly migrated through breaches in this layer to spread over the outer surface of iSLCOs, in a manner reminiscent of the cortical malformation cobblestone lissencephaly and animal models involving deficiency of as well as developing cortical basement membrane (Inoue et al. 2008).

### Inflation Promotes the Neural Stem Cell State and Inhibits Neuronal Differentiation

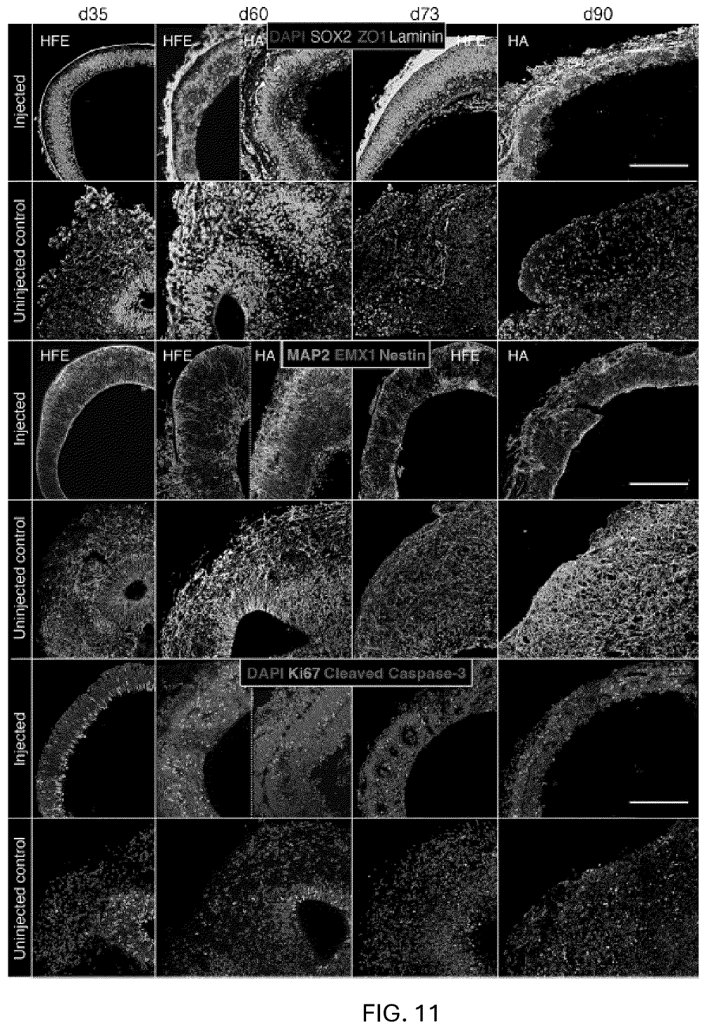

After day 50, while some iSLCOs continued to retain a pressurized lumen after d50, dropout from the iSLCO category due to spontaneous or accidental rupture continued, while organoids became increasingly opaque (FIG. 8D). To characterize the effects of ongoing pressure at these ages, iSLCOs from two batches, one HFE-injected and one HA-injected along with uninjected controls, were collected for sectioning and immunofluorescence analysis at 60, 75, and 90 days of differentiation. At these timepoints, ectopic rosettes similar to those observed between three and ten weeks in conventional organoids (Velasco, et al. 2019) appeared in the shell walls of all analyzed organoids, interspersed in some cases with retained stretches of persisting apicobasal organization (FIG. 11). However, these timepoints also revealed strong and continuing effects of injection on neuronal differentiation. To quantify nuclear marker expression over time, the inventors used Cellpose (Stringer et al. 2020) to segment nuclei in 20× images of immunolabelled organoids, and measured signal intensity in other channels using scikit-image (see Methods).

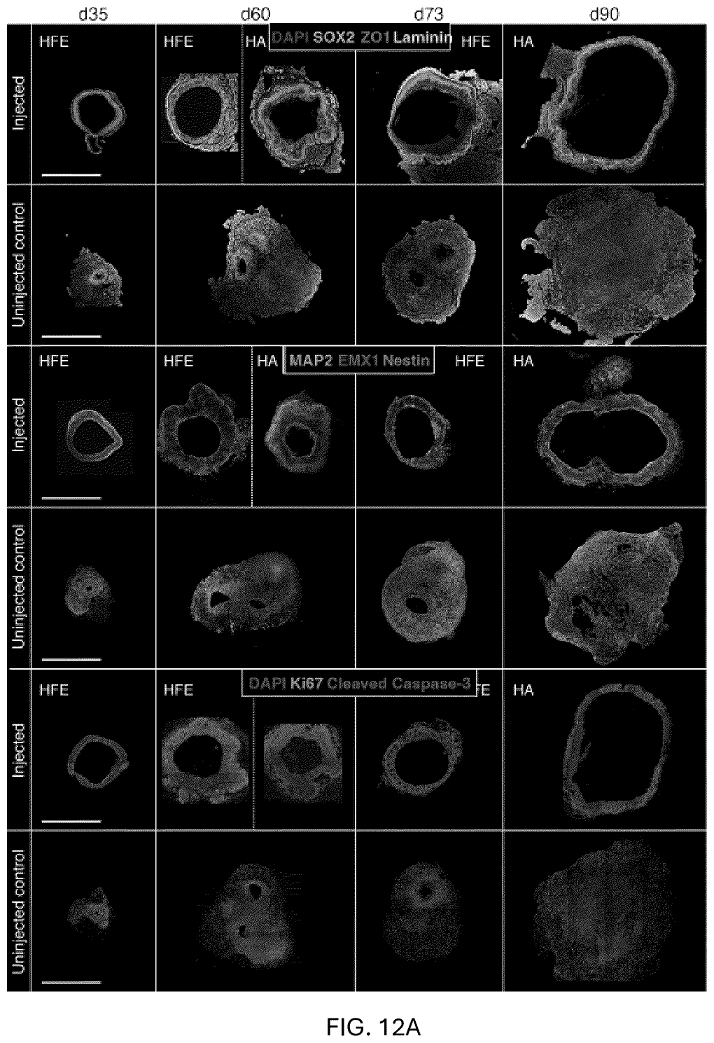

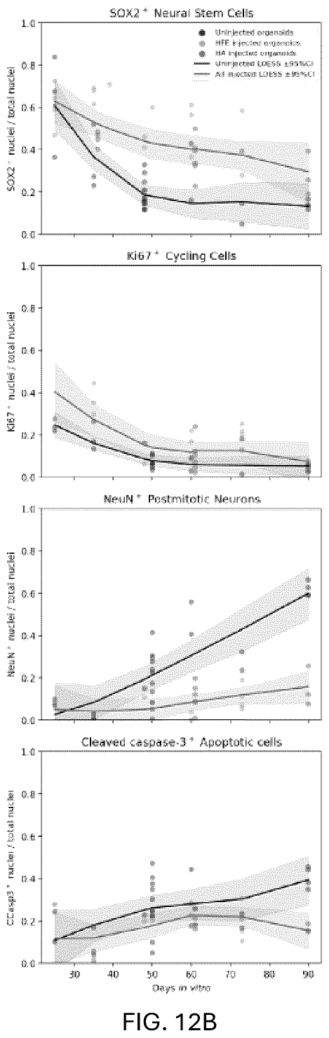

In control organoids, the proportion of nuclei expressing the early neural progenitor marker SOX2 decreased to approximately 20% of nuclei by day 50, remaining similarly sparse throughout the period examined. In contrast, iSLCOs injected with either HA or HFE showed a slower decay in SOX2-positive nuclei, with a mean remaining above 40% at 75 days of culture (FIG. 12B). A similar trend was observed for actively cycling cells, marked by Ki67; these cells represented a higher proportion of iSLCO nuclei at all timepoints through day 73. In contrast, differentiation of postmitotic neurons, detected by cell soma immunolabeling for the canonical neural marker NeuN, climbed steadily higher in uninjected controls, reaching over 60% of all nuclei at day 90; in iSLCOs, the increase in these cells was also consistent over time but much slower, remaining below 20% at day 90. This striking difference complements the observed changes in neural stem cell proportion, showing that pressure acts to alter the balance between maintenance of the stem cell pool and production of differentiated neurons in this in vitro model. Finally, reactivity for the cleaved form of caspase-3, an established marker for cells undergoing apoptosis (Uzquiano, et al. 2022), was abundant especially in organoid cores as organoids grew; however, it was consistently lower in inflated organoids, whose cores consisted of injected fluid rather than stressed tissue. To statistically test these effects, a median of three sections from each organoid (with a range of one to seven, depending on the availability of immunolabeled sections unobscured by folding, bubbles, or contamination) were imaged and automatically analyzed to annotate each nucleus as positive or negative for each marker. A hierarchical mixed linear effects model was used to account for variation between injection groups (uninjected vs. HA-injected and uninjected vs. HFE-injected); between different organoids; and between sections of the same organoid. The coefficients of these models representing the effects of injection with either HA or HFE were nonzero with high significance (p<0.001) for all of these comparisons, except for the difference in Ki67+ cells between uninjected and HA-injected organoids, which was not significant; however, in this and all other cases the difference between all pooled inflated samples and uninjected controls was significant at the p<0.01 level. The slope of the linear curve fit to mean positive proportion for each marker was also significantly different from 0 for each marker, showing that these effects of inflation could be detected across dynamically differentiating organoid groups. Because significant effects in the same direction are observed in almost all categories when injecting a simple biomimetic aqueous eCSF substitute or an inert non-aqueous fluid, and the difference between the HFE-injected and HA-injected groups was not significant, these effects can be inferred to result from the influence of stretching on the organized neuroepithelium rather than the specific biochemical composition of the fluid used.

Qualitatively, control organoids transitioned from expression of neuroepithelial markers SOX2 and ZO1 to express higher levels of the dorsal pallium progenitor marker EMX1 at 60 days of culture, followed by postmitotic neuron marker MAP2, which was highly abundant at days 75 and 90 of culture. In contrast, organoids that remained in the iSLCO group continued to show a neuroepithelial identity, as indicated by continued strong expression of SOX2, Ki67, and the neuroepithelial-specific apical junction marker ZO-1, with only few cells expressing more mature markers (FIG. 12A), although other than those described above, these markers were not quantified. Together, this IHC analysis shows that biomimetically organized organoids with continued intraluminal pressure undergo less neuronal differentiation than uninflated controls.

Spontaneous Loss of Intraluminal Pressure is Associated with Neurogenic Differentiation

To further explore the effect of inflation on differentiation, the inventors next asked whether artificial pressurization was primarily delaying neural differentiation, or alternatively changing the fate potential of neuroepithelial progenitors to make them incapable of generating neurons. The inventors therefore examined different cases in which pressurization was lost during culture, in order to assess whether neural stem cells in which neurogenesis was inhibited by inflation nonetheless retained cortical neurogenic potential that could be activated by subsequent deflation. First, the inventors examined injected organoids in which accidents during manipulation and reinjection caused spontaneous rupture of the shell wall. Expression of the dorsal pallium progenitor marker EMX1 as well as the postmitotic neuron marker MAP2 were strong in these organoids, comparable to uninjected controls, indicating that temporary inflation did not interfere with cortical neural differentiation.

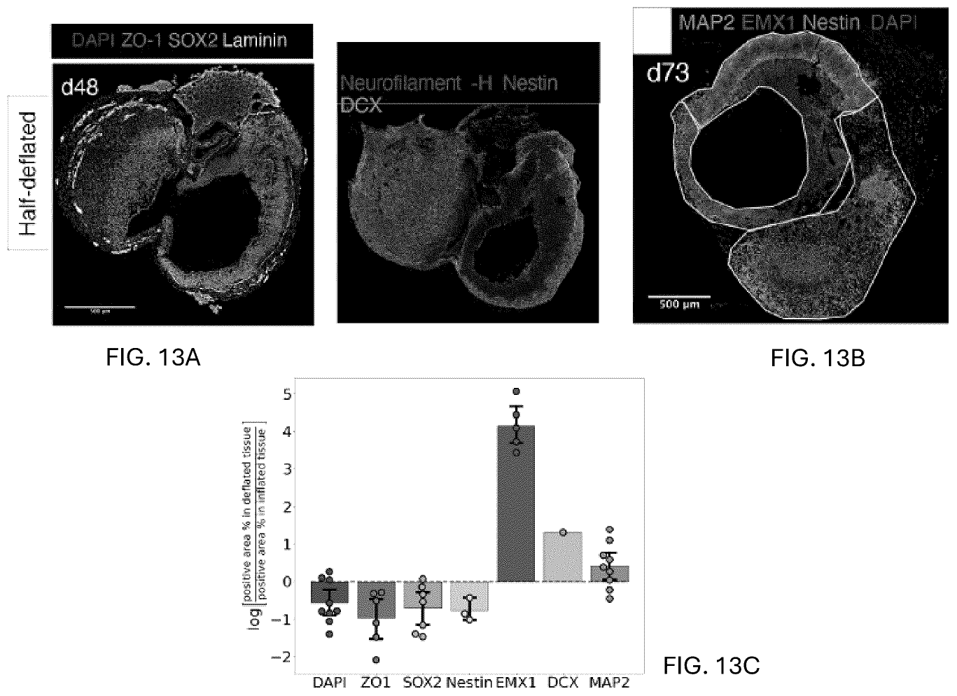

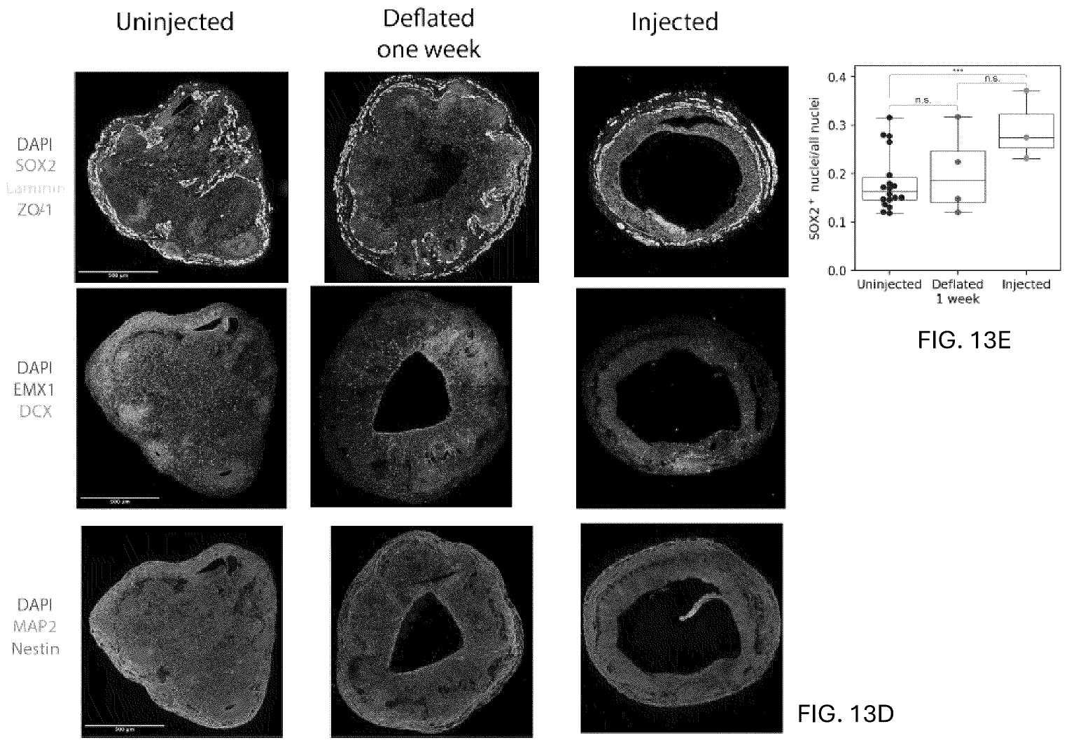

Separately, the inventors examined individual organoids that contained both inflated and deflated tissue due to anomalies in the inflation process. These serendipitous examples facilitated paired comparisons between the two types of tissue, with minimal confounding from any organoid-specific variation during culture or immunolabelling. In one approach, the inventors considered organoids that were formed by fusion of two injected single-lumen organoids that were placed into the same well after initial injection (FIG. 13A). For three such organoids from different batches, the inventors continued to inject both lumens until one spontaneously ruptured, then processed organoids for sectioning and immunofluorescence one week later, comparing the half of the fused organoid that visibly deflated with the intact half that continued to be stretched by internal fluid in brightfield images. In other cases, organoids retained overall organized single-lumen morphology, but had an isolated mass of tissue that grew from a locally disrupted region of the organoid wall, such as the wound left by an imperfect injection, and continued to grow without being exposed to the stretching force experienced by the main shell. The inventors similarly processed and immunolabelled such organoids one to three weeks after this outgrowth first appeared (n=3). For all these organoids containing both stretched and unstretched regions, the inventors sought to quantify whether the local loss of inflation allowed subsequent acquisition of neuronal identity.

For these analyses, the inventors manually traced the pressurized and non-pressurized regions of each organoid. The inventors then used ImageJ to manually set a single threshold for positive labelling across both halves of the image, and calculated the area with above-threshold signal intensity in each region of a single organoid (FIG. 13B). The effect of stretch was calculated by dividing the positive area fraction in the stretched tissue by the positive area fraction in the unstretched tissue. Half-inflated and outgrown organoids were pooled for statistical analysis. The results of this analysis (FIG. 13C) showed that deflation led to a significant reduction in nuclear density (DAPI signal), with a paired t-test p<0.05, consistent with growth of neuropil (non-nuclear tissue comprised primarily of cell processes) associated with neuronal differentiation. Deflated tissue also showed a significant reduction in expression of stem cell markers ZO1, SOX2, and nestin compared with adjacent still-inflated tissue (p<0.05). In contrast, neuronal marker MAP2 was well expressed in these organoids, with an insignificant trend towards higher thresholded area in the deflated tissue (p=0.057). In the only one of these organoids that was processed and immunolabelled for newborn neuron marker DCX, thresholded area was also strikingly higher on the deflated side. The cortical progenitor marker EMX1 was strongly increased in deflated tissue compared with adjacent inflated controls (p<0.01), consistent with differences in its expression between iSLCOs and uninjected control (FIG. 12A). This delayed expression was unexpected due to the early expression of this marker in vivo; for example, EMX1 is expressed in dorsal, but not roof-plate, pallium of the telencephalon shortly after neurulation in mice (Simeone et al. 1992) and chick (Frowein et al. 2006), as well as at timepoints as early as 23 days in conventional cortical organoids (Uzquiano et al. 2022). Although IHC labelling and quantification can be imprecise, the internal control used in this experimental design rules out the possibility that this variation is due to failure of antibody binding or technical errors during labelling and imaging. This data therefore indicates that inflation has the effect of delaying expression of EMX1, perhaps by maintaining stem cells in an early (pre-neurulation) neuroepithelial-like state, but this effect is reversed to allow robust EMX1 expression upon relaxation of pressure. Across all of these markers, loss of pressure-induced in-plane epithelial tension was followed by reductions in expression of neural stem cell markers and increases in neuronal markers. This shows that neuroepithelial stem cells were able to resume their neural differentiation trajectories following deflation, demonstrating that the inhibition of neurogenesis in single-rosette organoids by intraluminal inflation represents a delay in developmental timing rather than any irreversible alteration of cell fate.

### Immunohistochemical and Transcriptomic Analysis of Experimentally Deflated Organoids

The experiments described above rely on comparisons between uninflated controls and the subset of injected organoids that retained their inflated phenotype to advanced ages. Because only a small proportion of injected organoids remained in this category (FIG. 8), the inventors considered the possibility that this subset was selected for organoids that spontaneously progressed more slowly, resulting in increased retention of injected fluid, perhaps due to the greater integrity of apical junctions in young neuroepithelia (Aaku-Saraste et al. 1996; Veeraval et al. 2020). Because of this possibility, the causal direction between loss of shell integrity and rapid neurogenic differentiation could not be conclusively established by the above experiments.

To isolate the effect of pressure from any such selection effects, the inventors compared organoids that had been deliberately deflated at a determined timepoint with organoids from the same batch that continued to be inflated past this timepoint, as well as control organoids that were never inflated. For this, the inventors generated a cohort of inflated organoids and intentionally deflated a randomly-selected subset at culture day 42 (deflated group) while continuing to pressurize a second subset (reinjected group). The inventors then compared “deflated” and “reinjected” organoids one week later using IHC and single-cell RNA sequencing. To reduce the likelihood of accidental rupture, which would confound this analysis, these organoids were injected according to a more conservative schedule: whereas organoids in previous batches were reinjected whenever the shell thickness exceeded 100 μm until day 30 and 200 μm until day 60, this batch used a moving reinjection threshold intended to match the growth in thickness of the human cerebral wall, rising from 100 μm at initial injection to 500 μm at d49 (see Methods). The inventors applied this model to specifically examine how continued inflation affects organoid tissue architecture, differentiation, and molecular activity.

IHC analysis of injected, control, and experimentally deflated organoids one week after deflation confirmed a clear effect of intraluminal inflation on organoid histology. Deflated organoids formed many isolated rosettes, each having a small and isolated ZO-1-positive focus, in clear contrast to the continued unilocular tissue architecture of inflated organoids (FIG. 13D). At the cellular level, the more conservative reinjection schedule yielded inflated organoids with slightly higher expression of DCX and MAP2 than observed in previous batches, suggesting that the effects of inflation may be dose-dependent. Nonetheless, subtle differences could be observed between these inflated organoids and those in both the uninjected and deflated groups. The mean proportion of SOX2+nuclei was 10 percentage points lower in analyzed uninjected organoids (n=18) compared with iSLCOs (n=3) (p<0.05), and also 8 percentage points lower in experimentally deflated organoids (n=4), although the latter difference was not significant in this small sample (FIG. 13E; p=0.14). However, qualitative observations of increased DCX abundance and decreased nestin and ZO-1 in deflated relative to inflated organoids are consistent with a causative effect of inflation on inhibiting the differentiation of radial glial stem cells into newborn neurons in this model.

To explore the molecular effects of intraluminal pressure on progenitor development, proliferation, and differentiation, four deflated and four injected organoids were collected at day 49, one week after experimental deflation, for transcriptional profiling by single-cell RNA sequencing. This experimental design aimed to capture the differences in progenitor cell biology induced by pressure across the cerebral wall and reversed upon its loss, as most likely occurs during normal embryonic development upon establishment of communication between the ventricular system and subarachnoid space (Gato et al. 2020).

Following preprocessing with the CellRanger Multi pipeline and exclusion of unassigned, multiplet, and low-genomic-mRNA cells, the inventors recovered 40,043 cells, with 26,865 from deflated organoids, 13,178 from reinjected organoids, and 2234-10,542 cells assigned to each organoid. The inventors first verified that overall differentiation of these organoids was consistent with the results reported in the original protocol (Tidball et al. 2023). The organoids contained overwhelmingly cells with dorsal forebrain-like identities, with EMX2 expressed throughout. There was a clear gradient from cells expressing universal markers of neural stem cells (VIM, FABP7) to those expressing markers of newborn (DCX) and more fully differentiated neurons (STMN2).

The inventors found strong representation of the core cortical excitatory lineage from radial glia through the early-born cortical projection neurons expected at this age. This branch of the dataset expressed FOXG1 throughout, and contained abundant cortical progenitors positive for VIM, FABP7, and EMX2, along with more specific cortical stem cell markers PAX6, SFRP1, and LHX2. EMX1 expression was found throughout the sample and enriched in the cortical lineage, consistent with its expression pattern in developing mouse brain (Simeone et al. 1992), although at relatively low levels, consistent with its low level of detection in immunolabeled injected organoids as described above. Subpopulations of these cortical progenitors expressed CRYAB and HOPX along with other markers of ventral and outer radial glia, as well as markers of actively cycling cells in G2/M phase (MKI67) and S phase (PCNA). The inventors also detected a relatively small population of neuronal intermediate progenitor cells expressing EOMES, along with migrating neuron markers such as NHLH1. Finally, this branch terminated in developing excitatory deep-layer projection neurons, expressing NEUROD6 and NEUROD2 along with markers for neurons of layers 6 (FOXP2 and TLE4), 5 (BCL11B), and 4 (FEZF2).

In addition to this branch representing the cortical excitatory projection neuron lineage, and consistent with the original publication (Tidball et al. 2023), the inventors found a large population of putative roof plate and cortical hem cells. This is likely due to the use of the artificial WNT agonist included in the original protocol to promote growth and reduce death, which has been previously observed to imitate roof-plate signaling to induce cortical hem identities (Tidball et al. 2023; Amin et al. 2023). A large population of these cells expressed radial glial markers (VIM, FABP7) along with RSPO2 and LMX1A, which are strongly and specifically expressed in the cortical hem and roof plate in mice (Watanabe et al. 2023; Iskusnykh et al. 2023). Automated label transfer using the Human Neural Organoid Cell Atlas (He et al. 2024) suggested diencephalon and midbrain identities for these cells. However, they were negative for all diencephalon and midbrain markers identified by Braun et al.

2023, such as GSX2 and EN2, except for those known to be associated with cortical hem. The inventors therefore speculated that these assignments can be explained by the limited sample dissection resolution that was possible in the original underlying human embryonic atlas, which did not distinguish cortical hem from the closely physically adjacent diencephalonl. Further supporting a cortical hem identity, these progenitors were associated with three groups of more differentiated cells that derive from this lineage in vivo. The first was a small population of cells expressing TTX, a specification marker for the choroid plexus, which originates in the telencephalon from the medial most region of the cortical hem (Saunders et al. 2023). The other two branches terminated in cells with highly abundant expression of RELN along with generic neuron markers STMN2 and NEUROD6, which the inventors assigned as Cajal-Retzius cells. One of these branches expressed canonical markers of medial-derived CR cells including LHX1 and TP73 (Elorriaga et al. 2023), while the other, more strongly continuous with the cortical hem progenitors, expressed RELN along with GRM1 (mGluR1), which marks a less-well-understood population of Cajal-Retzius cells known as “lot” cells, whose origins have been controversial (Ruiz-Reig et al. 2017). This result supports a cortical hem-like origin for at least some of these cells during human development.

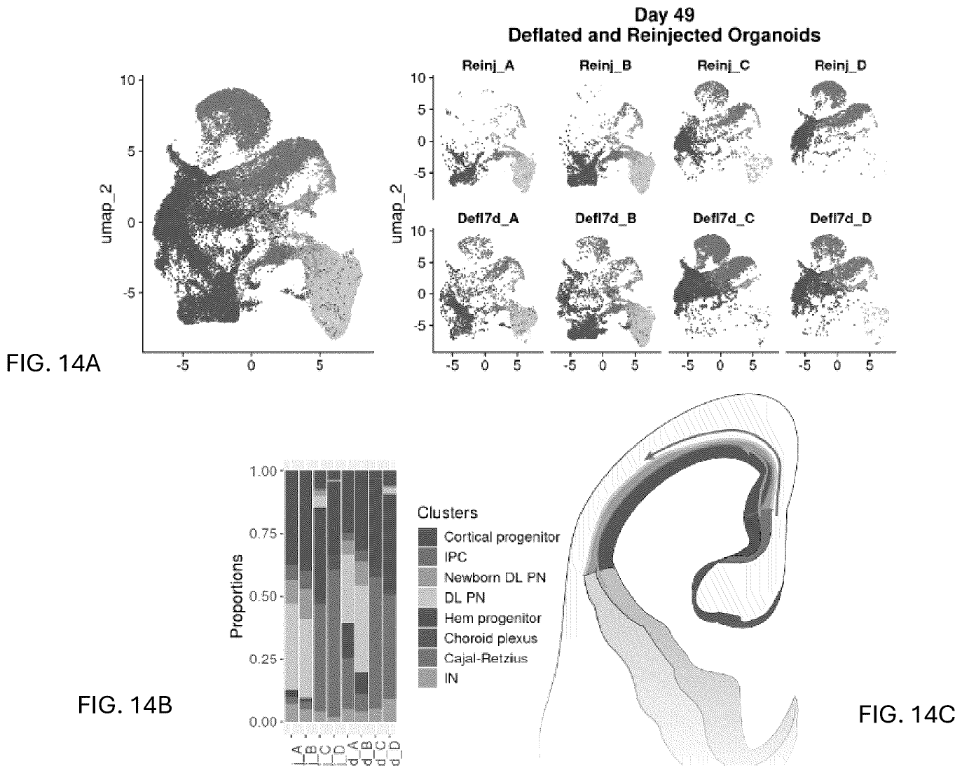

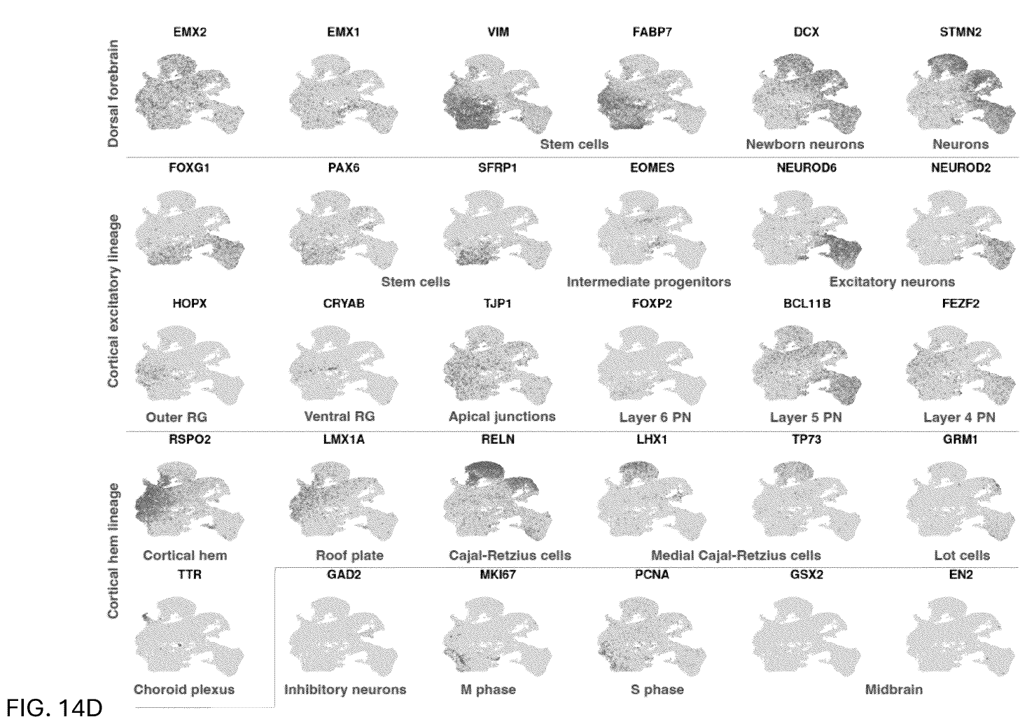

In the publication that first developed the single-lumen organoid protocol adapted, these two lineages—cortical and cortical hem—were found to be present in consistent proportions in each organoid, beginning at the earliest sequenced timepoints at one month. Notably, these organoids showed compromised unilocular architecture at this timepoint, with initial single rosettes breaking into multiple germinal zones as organoids continued to grow (Tidball et al. 2023). The inventors observed above that the persisting single continuous germinal zones found in iSLCOs appeared to express transcription factors such as SOX2 more evenly around the entire single-rosette organoids compared with uninjected controls that formed multiple rosettes (FIG. 10), an observation consistent with recent reports of greater progenitor homogeneity within individual rosettes of multi-rosette organoids (Chiaradia et al. 2023; Singh 2024). The inventors therefore hypothesized that iSLCOs that were preserved as single rosettes for longer would show less variability in trajectory choice within a single organoid. To test this prediction, the inventors examined cell type proportions in the eight organoids sequenced, following demultiplexing of labels assigned to each sample using the 3′ CellPlex Kit for Cell Multiplexing (10× genomics). Consistent with the prediction, the inventors found that each organoid was dominated by either the cortical or cortical hem lineage, and that this compositional unevenness was stronger in the continuously inflated organoids than in those that were deflated on day 42 (FIG. 14). Two organoids in each group consisted of than 80% cells from the cortical hem lineage, while the remaining two consisted of more than 80% cells from the cortical lineage, except for one of the deflated cortical organoids which contained closer to 60%. These results add support for the hypothesis that maintenance of single-lumen morphology facilitates generation of organoids with more homogenous intra-organoid identity classes. The inventors next compared individual cell type proportions between the two primarily-cortical organoids from each biological group. The inventors found that deflated cortical organoids contained a slightly lower proportion of cortical RGs, IPCs, and newborn deep-layer projection neurons and a higher proportion of mature deep-layer projection neurons, consistent with results obtained from IHCC quantification in the full timecourses, but these differences were not significant (FDR>0.05)).

### Molecular Mechanisms of Inflation-Driven Biological Differences.

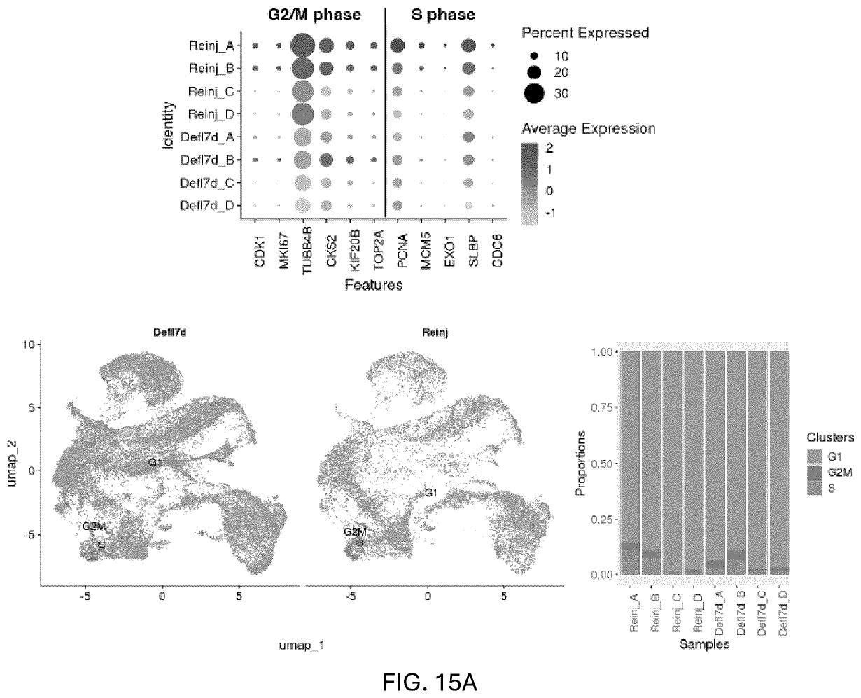

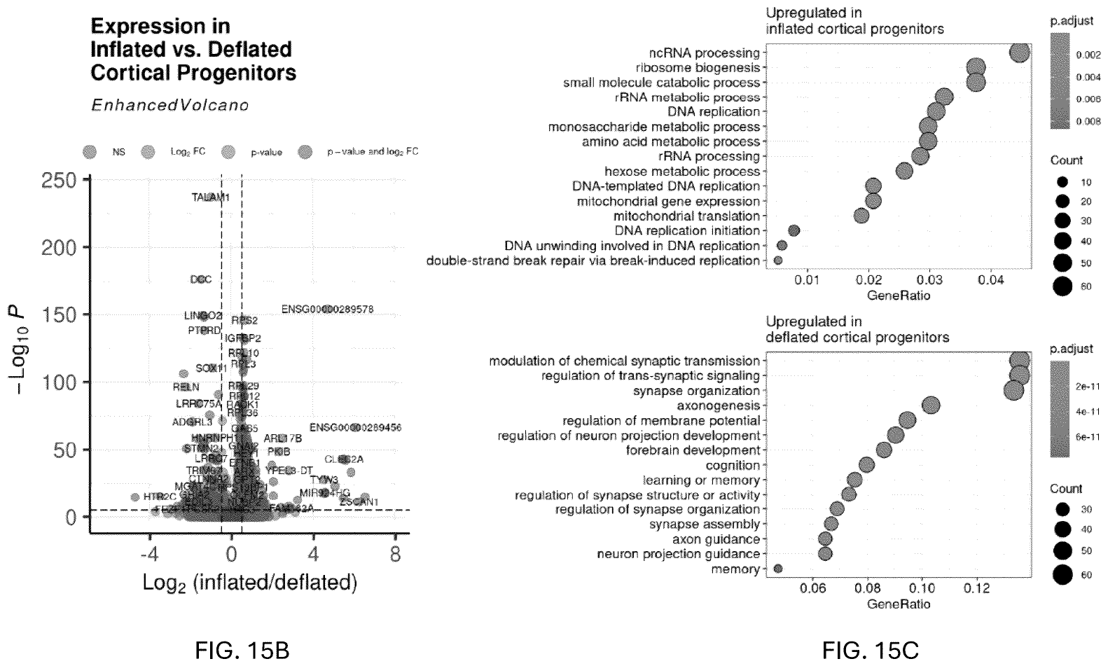

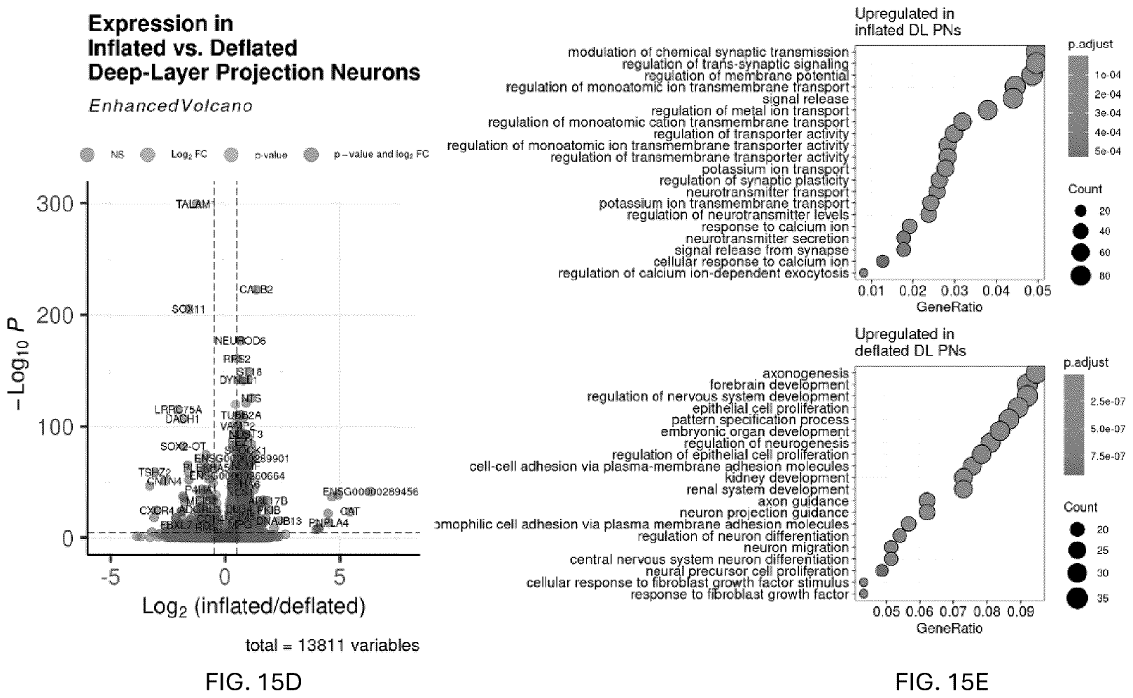

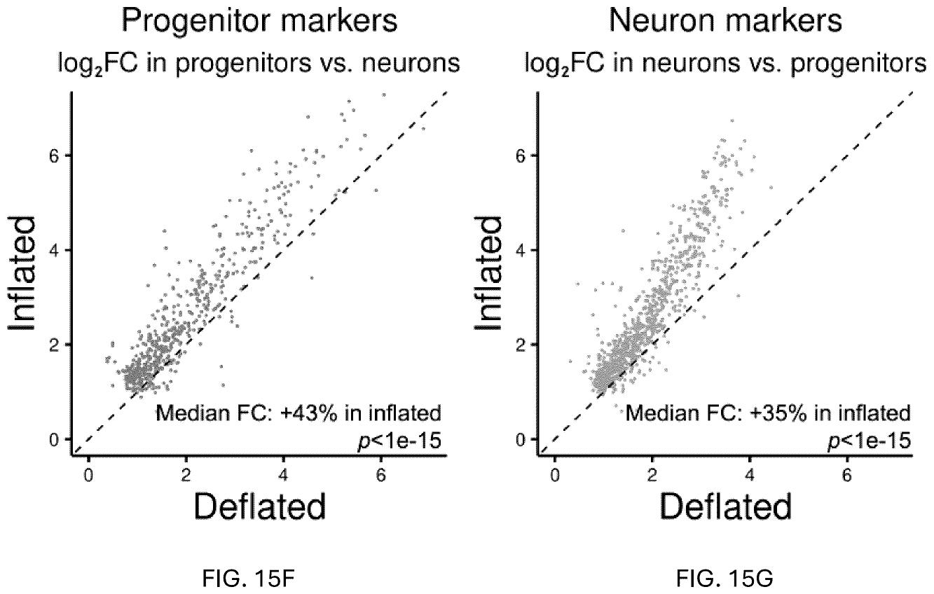

The inventors next sought to determine whether the previously observed effects of pressure on cell cycling could be detected in this dataset. To provide a direct comparison to earlier work counting Ki67-positive nuclei (FIG. 12) the inventors compared expression levels of a list of canonical markers of the G2/M phase of the cell cycle (including MKI67) and of S phase (including PCNA). All of these markers were significantly upregulated in reinjected organoids (FIG. 15A; p<0.05 for PCNA, p<0.0001 for all others, Wilcoxon non-parametric test). To estimate the proportions of actively cycling cells, cortical and cortical hem progenitors were subclustered, similar markers were used to identify subclusters containing cycling cells, and the proportion of cycling cells as a fraction of all organoid cells was compared between inflated and deflated groups. A larger fraction of cells in reinjected organoids (7.3% vs 5.7% in deflated organoids) were actively cycling, although this change was not statistically significant when comparing at the level of organoids (FIG. 15A). Overall, this evidence supports the findings described above, showing that ongoing intraluminal inflation promotes residency in a mitotic state, and slightly extends them to show that this effect includes both M and S phases of division.

Interestingly, the use of sample multiplexing and the high purity of cortical vs. cortical hem identity in individual organoids, as discussed above, made it clear that cycling cells were overwhelmingly found in the two organoids from each condition that retained a cortical identity. This finding suggests that, at this age, organoid cortical hem progenitors are primarily differentiating or quiescent rather than actively proliferating like a subset of their pallium-like progenitor counterparts.

Next, the inventors sought to describe the molecular effects of pressure on neural differentiation in organoids. The inventors used Seurat to inspect genes differentially expressed between inflated and deflated cells of specific cell types, and applied gene ontology analysis to identify shared pathways affected by the loss of intraluminal inflation. Consistent with the observed effects on mitosis, inflated cortical progenitors were relatively enriched for genes associated with DNA replication and other housekeeping genes, a pattern that was unchanged when ribosomal and mitochondrial genes were removed from the enrichment list. In contrast, deflated cortical RG had higher expression of genes related to the differentiation and function of mature neurons, including GO terms related to synaptic signaling, neurite and axon growth, and high-level functional terms such as “cognition” and “memory”. These comparisons offer direct evidence that loss of intraluminal pressure induces differentiation towards a neuron-like fate in cortical radial glia, at the expense of proliferation-related activity. To confirm that these effects were not driven by irregularities in cell type assignment between conditions, the merged object was clustered, and clusters corresponding to core cortical progenitors were split into deflated and inflated subsets to repeat DEG analysis, giving almost identical results.

While progenitors showed signs of accelerated maturation in deflated organoids, the opposite effect was observed when inspecting DEGs in postmitotic projection neuron clusters. Inflated projection neurons were strongly enriched in GO terms related to mature neuronal function, including synaptic neurotransmitter release, transmembrane ion transport, and response to calcium signaling. Conversely, deflated PNs were enriched in terms related to the proliferation and differentiation of neural stem cells, including “neural precursor cell proliferation”, “regulation of epithelial cell proliferation” “regulation of neurogenesis”, “regulation of neuron differentiation”, and “forebrain development”. In summary, neurons and neuronal progenitors were more distinct from each other in inflated organoids, with less neuronal activity in progenitors and less progenitor activity in neurons, relative to organoids that were in the process of losing their biomimetic organization after only a week of deflation. Thus, inflation enhanced or prolonged the specificity of neuron and progenitor cell classes, reducing biological overlap between these identities within individual cells.

Finally, the inventors sought to identify molecular signaling pathways responsible for the changes the inventors saw in this data, and by extension for the aligned overall trends the inventors previously observed throughout the brightfield and immunohistochemical results. To do this, the inventors used gene set enrichment analysis to examine genes that were differentially expressed within cell clusters across inflation treatments (see Methods).

Across cell types and individual organoids, the “YAP1_ChEA” module was upregulated in inflated relative to deflated conditions with high significance, although the effect size was small (0.2<Cohen's D<0.5) in some cell types, including cortical progenitors. This gene set comprises 2212 genes identified in functional studies whose chromatin accessibility is altered by YAP/TAZ signaling; smaller gene sets containing dozens of genes involved in YAP/TAZ signaling did not show a comparable effect. Another mechanotransduction pathway, representing focal adhesion-induced upregulation of cell proliferation through focal adhesion kinase activation of the PI3K-Akt-mTOR-signaling pathway, was disproportionately upregulated in stem and intermediate progenitor cells; this finding supports the observation of Desmond et al. 2014 that an increase in intraluminal pressure shifts phosphorylated FAK to the apical surface of chick embryonic brains (FIG. 15C). Because both of these signaling pathways are known to promote mitosis, they represent complementary possible mechanisms for the prolongation of neural stemness that the inventors observe.

The inventors also found that inflated progenitors, but not neuronal cells, more strongly expressed hallmark genes of cell stress (hypoxia and glycolysis), amplifying an underlying trend towards greater expression of these modules in progenitors (FIG. 15C). The inventors speculate that these differences are explained by the breakdown of biomimetic tissue organization in deflated organoids (FIG. 10). Because progenitors in deflated organoids became intermingled with neuronal cells throughout the full thickness of the collapsing organoid, they are exposed both to hypoxic conditions deep in the organoid and to more superficial conditions. In contrast, stem cells in inflated organoids not only experienced increased mitotic activity, increasing cell stress, but remained deeply buried at the core of the organoid (FIG. 13), where single-cell transcriptomics (Uzquiano et al. 2022) and immunolabeling data (Qian et al. 2020, FIG. 12) have shown progressively increasing cell stress as organoids grow beyond the oxygen diffusion limit.

In addition to these signals, unbiased screening consistently identified increased expression of metabolic genes relating to fatty acid metabolism and its regulation in progenitors subjected to inflation (FIG. 15C). Specifically, hallmark pathways of fatty acid metabolism and cholesterol homeostasis were often upregulated in cortical progenitors and other cells of inflated organoids, including in systematic pairwise comparisons between organoids in each group. Finally, small changes (Cohen's D below 0.5) in canonical Wnt and TGBβ signaling activity were observed in some cell types, generally showing increases in the activity of these signaling pathways in deflated organoids.

### Discussion

### Summary of Results: Intraluminal Fluid Pressure Regulates Morphogenesis and Neurogenesis in Organoids.

Although human cortical organoids are a powerful model for experimental investigation of human neural development, their recapitulation of in vivo neuroglial differentiation trajectories has not been matched by a similar degree of fidelity to the morphology of the developing cortex (Pagliaro et al. 2025). The inventors report a novel variation on organoid culture that artificially recreates the pressure-driven inflation of the telencephalic vesicles, which is necessary in embryonic brains to prevent the development of organoid-like ab normal tissue architecture. This protocol yields larger, older, and more biomimetic single-lumen cortical organoids than have previously been reported. These inflated single-lumen cortical organoids (iSLCOs) maintain tissue architecture closely resembling that of the developing mammalian neocortex for up to 1.5 months, with single ventricle-like structures reaching over a millimeter in diameter without disruption by ectopic rosettes. The inventors characterize the progression of neural differentiation in these models and find that ongoing inflation strongly inhibits the neurogenic differentiation of neural stem cells in favor of increasing mitotic proliferation. Importantly, these organoids still demonstrate rapid acquisition of neuronal identity following release of intraluminal pressure. The inventors provide single-cell transcriptomic data that further illuminates the effects of inflation and deflation on cell cycling, cell type composition, and specific molecular pathways likely to be important in transducing tangential stretch into altered progenitor behavior. These results demonstrate that intraluminal inflation is a biologically and technically important feature missing from existing organoid protocols and provide a proof-of-principle approach for improving organoid fidelity to the embryonic brain by restoring this key physical process.

### Intraluminal Inflation as a Tool for Organoid Bioengineering.

The improved architecture and biological effects observed in the inflated organoids point to a new avenue for future organoid bioengineering efforts. The inventors propose that intraluminal inflation can be applied to emulate the structural development of the human cortex in vitro by emulating the physical forces that drive early neural tube expansion. This approach could be particularly useful to investigate biological phenomena that depend on normal biomimetic organization. For example, research on the mechanisms and molecular guidance of radial migration along the radial glial scaffold, a complex process that underlies diverse clinically important defects of corticogenesis, depends on the availability of experimental models that robustly produce such scaffolds (Ferent et al. 2020). This research could be facilitated by the use of organoids such as those the inventors report here, with well-organized, highly anisotropic radial glial scaffolds (FIG. 10). The large-scale and often organoid-wide biomimetic organization in these organoids contrasts with the more sporadic development of impressively well-organized stretches in isolated regions of conventional cerebral organoids (Lancaster et al. 2017), and could allow more sensitive detection of deviations from this organization. In addition, the observed reduction of apoptotic nuclei at the latest timepoint and across the course of differentiation indicates that biomimetic inflation helps to address one of the major limitations of current organoid models. Inflation might partially replace the necrotic core typical of 3D in vitro models, which has been shown to be enriched for metabolically stressed cells and cells with low similarity to endogenous fetal cell types, with an experimentally tunable bioinert or biomimetic fluid, enhancing the cellular as well as structural similarity of these models to the embryonic brain.

Many important differences remain between iSLOs and embryonic brains, including the lack of vascularization. Notably, the inventors found that hallmark genes of cell stress (hypoxia and glycolysis), were relatively upregulated in inflated progenitors, but downregulated in inflated neuronal cells, more strongly expressed amplifying an underlying trend towards greater expression of these modules in progenitors (FIG. 15C). The inventors speculate that these differences can be explained by the breakdown of biomimetic tissue organization in deflated organoids (FIG. 10). Because progenitors in deflated organoids became intermingled with neuronal cells throughout the full thickness of the collapsing organoid, they are exposed to hypoxic conditions deep in the organoid along with more superficial conditions. In contrast, stem cells in inflated organoids not only experienced increased mitotic activity but remained deeply buried at the core of the organoid (FIG. 13). Conversely, neurons in the inflated organoids migrated appropriately to a dedicated mantle layer on the organoid surface, leading to reduced hypoxic and glycolytic stressed compared with neurons in the deflated organoids that were distributed throughout the organoid. To maximize the long-term value of this approach, future bioengineering approaches could explore ways to actively turn over the fluid inside a biomimetically polarized cyst or tube, alleviating the nutrient and oxygen stress experienced by these progenitors realistically concentrated at the deepest part of the organoid.

The interaction of stem cell stress and tissue architecture in the iSLCO model provides new insights into the behavior of neural organoid cells during differentiation. The single-cell differential gene expression analysis suggests that these better-organized organoids, with clear separation of a single germinal zone from a mantle containing postmitotic neurons, also yields cells with greater separation of activities normally distinct in in vivo cells. Relative to deflated organoids that more closely resemble conventional organoids, inflated neural progenitor express fewer genes associated with later neuronal development and function such as neurite outgrowth and synaptogenesis. Conversely, inflated organoid neurons at only 49 days of differentiation more highly express genes associated with mature neuronal functions such as neurotransmitter release and action potential discharge, while expressing fewer genes associated with neural progenitor proliferation and differentiation (FIG. 15B). These observations are consistent with past results showing reduced cell type specificity along with cell stress in at least some brain organoid cells (Bhaduri et al. 2020). This phenotype was found to be reduced by xenotransplantation of organoids into animal brains (Bhaduri et al. 2020), an approach that has gained popularity in recent years (Pagliaro et al. 2025). However, the results indicate that cell type specificity can alternatively be enhanced by improving the architectural similarity of organoid tissue to that of the embryonic brain, while retaining many of the advantages in accessibility and experimental control that come with in vitro culture. The immunohistochemical analysis indicates that apoptosis was overall reduced by replacing organoid cores with fluid, but in the 49-day sequencing experiment glycolysis and hypoxia signatures were slightly higher in progenitors of inflated organoids that showed enhanced cell type specification. Accordingly, the spatial intermingling of progenitors and neurons in organoids, leading to perturbation of the distinct neural stem cell and cortical plate niches, may play a larger role than cell stress in generating unwanted organoid-specific phenotypes.

In this study, the inventors highlight the mechanical effects of organoid injection, showing similar biological results from using either fluorinated oil (HFE) or a simple eCSF substitute composed of hyaluronic acid in aqueous solution (FIG. 12).

The effectiveness of the latter approach, with observed retention of injected macromolecules for multiple days (FIG. 8 and FIG. 9E), suggests that in the future this approach could be used to repeatedly expose human neural progenitors to water-soluble factors found in the eCSF, or a cocktail or progression of such factors. Early eCSF is abundant in such signaling factors, has strong effects on progenitor biology that depend on specifically being exposed to the primary cilia that project into the ventricular cavity (Lehtinen et al. 2011), is temporospatially dynamic (Saunders et al. 2023), and can be challenging to manipulate in vivo (Jang and Lehtinen 2022). In addition, diffusion from injected intraventricular fluid through a biomimetically organized, apicobasally polarized shell could facilitate the generation of apicobasal concentration gradients of molecules of interest, which are thought to be important in radial migration and progenitor and neuronal identity (Ferent, et al. 2020). Therefore, this functionality in iSLCOs could be a valuable addition to the tools available for experimentally studying the biological effects of eCSF and other apicobasally-restricted signals on human neural progenitors.

The apparent alteration of developmental tempo in iSLCOs by addition of biomimetic pressure raises the question of how the timing of neural development in human embryos compares with that in standard organoid models. Although the sequence of key differentiation events in both cases has been shown to be conserved (Gordon et al. 2021; Uzquiano et al. 2022), surprisingly little work has systematically compared the timing of these milestones from a quantitative or scaling perspective. One study did perform such a meta-analysis, using stage-specific genes from human fetal references to reanalyze published data from organoids of different ages and assign them to a matching fetal age. This paper found a highly significant correlation between organoid and fetal age with r=0.89 (Tanaka et al. 2020). The reanalysis of the data shown in this report suggests that this linear regression predicted organoids' “fetal age” as 50 days plus 0.55 times the organoid age in days; this equation suggests that organoid differentiation is accelerated at early stages, perhaps due to the use of small molecules to rapidly pattern neural fates, followed by slower development over time and a failure to reach fully mature stages. Consistent with this model, a more comprehensive analysis using many more organoid protocols and updated fetal data recently found that organoids between 1 and 3 months matched strongly with first-trimester fetal data, but older organoids up to 10 months were evenly divided between molecular signatures of first- and second-trimester fetal brain, with under 5% of cells at even the latest timepoints matching third-trimester data (He et al. 2023). Overall, these comparisons can be interpreted as indicating that organoids initially mature faster than embryonic tissue, but later fail to form late-born cell types appropriate for their temporal age. The pressure mechanism the inventors report provides a straightforward explanation for this pattern. The inventors observe that pressure delays neurogenesis, while deflation accelerates it relative to inflated control tissue, potentially including the inflated embryonic brain. Therefore, the inventors propose that the absence of normal intraventricular pressure in organoids from the earliest ages causes progenitors to undergo premature differentiation, leading to rapid depletion of the progenitor pool before progenitors acquire competence to produce later-born cell types. Subsequently, aberrant tissue organization interferes with the structured cellular crosstalk that promotes the maturation of both stem cells and their neuronal progeny in vivo Di Bella et al. 2024 MakingCortex, Stoufflet et al. 2023 ShapingCrosstalk, leading to deviation from the temporal progression of normal corticogenesis.

Notably, in the iSLCO model, the thickness of the ventricular zone and mantle at day 50 of culture closely matched that of the human embryonic telencephalon at the same age (Ze cevic 1993; Bayer and Altman 2008). Similarly, at this timepoint iSLCOs continued to contain abundant SOX2+ neural stem cells (over 40%) and few differentiated neurons (under 10%); these proportions closely matched those found the dorsal forebrain lineage of human embryos at the matching timepoint seven weeks after conception, which was found to be over 40% SOX2+ radial glial stem cells in a recent single-cell atlas (Braun et al. 2023). In contrast, uninjected controls at this timepoint had under 20% stem cells and over 20% differentiated neurons, suggestive of accelerated development relative to the human embryo. However, at ten weeks of development—after the medial aperture has likely opened—this radial glial proportion in embryos drops to just over 20% while neurons reach almost 40%; in the study, these figures more closely match deflated controls at day 73, rather than the iSLOs, which remained above 40% SOX2-positive at this timepoint (FIG. 12). While these observations are relatively superficial, they provide supporting evidence for the proposal that timing inflation and deflation of organoids to match that of the embryonic brain could be an effective strategy for temporally aligning the sequence of neural development in organoids with in vivo human brains. Further research should test these hypotheses by investigating whether inflating iSLCOs past the time when abundant neurogenesis begins in conventional organoids, then allowing gradual loss of pressure over additional months of culture, yields organoids with more mature molecular signatures and neuroglial populations.

### Biomimetic Physical Forces in Self-Organization of Cortical Organoid Tissue Architecture.

The controlled experiments, analyzing injected single-lumen organoids alongside uninjected as well as experimentally deflated organoids from the same batch, directly demonstrate that intraluminal inflation strongly promotes the preservation of tissue architecture. This result adds to a growing literature investigating the effects of physical forces on cortical development using in vitro models. Recent work has explored the effect of embedding brain organoids in matrices with varying viscoelastic properties on their growth and development, finding an optimal stiffness for neuroepithelial cyst morphogenesis (Ranga et al. 2017); two papers using slightly different techniques for encapsulating suspension-cultured organoids with alginate hydrogels reported either reduced organoid and rosette size (Camps et al. 2022) or increased progenitor number and rosette size along with accelerated maturation (Tang et al. 2023). To directly isolate the effects of mechanical force on neural stem cell identity and differentiation in organoids, a recent study acutely compressed 30-day cerebral organoids by 40-60% of their original height. This mechanical strain increased immunolabelled SOX2 signal in organoid rosettes 24 hours later, and reduced the rate of differentiation into neurons of progeny born at this time. Single-cell analysis after 24 hours identified changes in metabolic features, but little difference in differentiation trajectories was detected after this brief interval. Similar compression of 60-day organoids did not affect SOX2 expression (Lampersperger et al. 2025). While this work represents an important advance in understanding the role of mechanical forces in early brain development, acute compression of disorganized organoid tissue likely differs in important ways from the chronic tensile loading of a realistically organized neuroepithelium. Notably, adherens-junction-mediated cell-cell signaling like that which preserves NSC identity (Jianing Zhang et al. 2010), is mechanotransduced in epithelia exposed to tensile strain, with very different effects at the cellular level compared with cells under compressive loading (Campàs et al. 2023). Therefore, recapitulation of long-term biomimetic tensile strain on organized neuroepithelial tissue represents an important advance for in vitro investigation of mechanobiology in cortical development.

Several mechanisms could contribute to the identified effect of pressure on the self-organization of normal, unilocular histology in cortical organoids. One strong possibility is that the failure of organoids to spontaneously inflate results in physical overcrowding of nuclei at the apical surface as neuroepithelial stem cells proliferate, forcing them to delaminate from the lumen surface and reorganize in ectopic rosettes. This possibility is supported by a series of results obtained from studying the early mouse ventricular zone, showing that actomyosin contractility at the apical surface pulls NESCs towards each other, (Okamoto et al. 2013), resulting in high compression of nuclei at the apical surface of the VZ as measured by atomic force microscopy (Nagasaka et al. 2016), and that this compression contributes to the expulsion of newborn neurons from the VZ (Shinoda et al. 2018). This compression force appears to be capable of driving stem cell delamination to the basal layer when apical overcrowding is induced by shortening apical processes (Okamoto et al. 2013), or inducing excessive rapid progenitor division (Chenn and Walsh 2002; Lavado et al. 2018; Saito et al. 2018; Miyata 2023), as in other epithelia (Pinheiro and Bellaïche 2018). The inventors find that nuclei in uninjected single-rosette organoids are increasingly compressed, and that delamination occurs at a threshold value of nuclear aspect ratio close to the maximum value which preceded neuroepithelial buckling in an earlier organoid model of cortical folding (Karzbrun et al. 2018), suggesting that it may represent an approximate limit for nuclear compression in neuroepithelial tissue. Therefore, the inventors speculate that inflation prevents the formation of ectopic rosettes largely by spreading nuclei apart at the apical surface, preventing overcrowding-induced delamination of neuroepithelial stem cells with structural plasticity that gives them rosette-forming potential. A complementary possibility is that epithelial stretch could induce remodeling at apical junctions, as has been observed in tight and adherens junctions of many epithelia (Cavanaugh et al. 2012; Pinheiro and Bellaïche 2018). Such remodeling could potentially explain the persistence of ZO-1 at inflated apical surfaces (FIG. 10); the rapid disassembly of neuroepithelial structure following one week of inflation, which is less readily explained by nuclear compression (FIG. 13); and even downstream effects on cell identity, which can be triggered by stretch-induced changes in adherens junction signaling (Campàs et al. 2023). However, ZO-1-positive tight junctions are generally reduced and made less continuous by tissue stretching in other model epithelial systems (Varadarajan et al. 2019), while in this model the presence of these junctions is increased in the presence of inflation-induced stretch. This indicates that, if this effect contributed to the stabilization of tissue architecture by intraluminal inflation, different mechanisms are likely involved.

In addition to these physical effects of inflation in in vivo and in vitro neuroepithelial histogenesis, it has been shown that the developing mammalian brain experiences elastic compression from the overlying calvarial tissue, contributing to its morphogenesis (Miyata 2023). This model incompletely imitates this effect. Basement membrane is incorporated in iSLCOs through incorporation of basement membrane deposited by dissolved extracellular matrix in the culture media, similar to other approaches in organoids (Kadoshima et al. 2013; Martins-Costa et al. 2023). Even when a higher concentration of 2% Geltrex was tested, the resulting layer often became discontinuous and overgrown by migrating neuroblasts at later ages, appearing to provide minimal if any compressive force (FIG. 10). Therefore, the lack of external compression from a continuous pial membrane in this project represents a limitation in face validity, and may also represent a practical limitation on the longevity and stability of radial glial organization. Recent bioengineering progress in this area have attempted to replicate biomimetic external compression by embedding brain organoids in an elastic hydrogel shell (Camps et al. 2022; Tang et al. 2023), or, in an approach particularly complementary to that developed in this project, by coating uninflated single-lumen neuroepithelial cysts with a layer of meningeal fibroblasts to actively maintain an external basement membrane while secreting basally localized signaling factors (Jalilian and Shin 2023). These approaches could potentially be productively applied in combination with lumen inflation for further investigation of the interplay of realistic mechanical forces from inside and outside the cerebral wall during cortical morphogenesis.

### Possible Mechanisms and In Vivo Relevance of Prolongation of Neural Stemness by Intra-Luminal Pressure.

The single-cell data identifies the pro-mitosis effect of pressure that has previously been described in embryo models (M. Alonso et al. 2000; Desmond et al. 2005), in addition to signatures consistent with the cell type composition effects the inventors observe using immunolabeling analysis (FIGS. 12 and 15). The molecular mechanisms by which pressure affects brain histology and differentiation remain an important open question that the data do not conclusively resolve. However, the data the inventors have generated, comprising 40,000 cells exposed to different inflation treatments and differing only in one week of intraluminal pressure exposure, provides important indications of possible pathways involved in this effect. Because the role the inventors propose for intraluminal pressure in regulating the timing of differentiation is novel, minimal literature has explicitly discussed possible molecular mechanisms for transducing the physical forces of lumen inflation into altered behavior of neural progenitors. However, many pathways that are known to play a role in cortical development have also been reported to respond to mechanical regulation. Below, candidate molecular pathways are discussed that could effectuate this mechanobiological process.

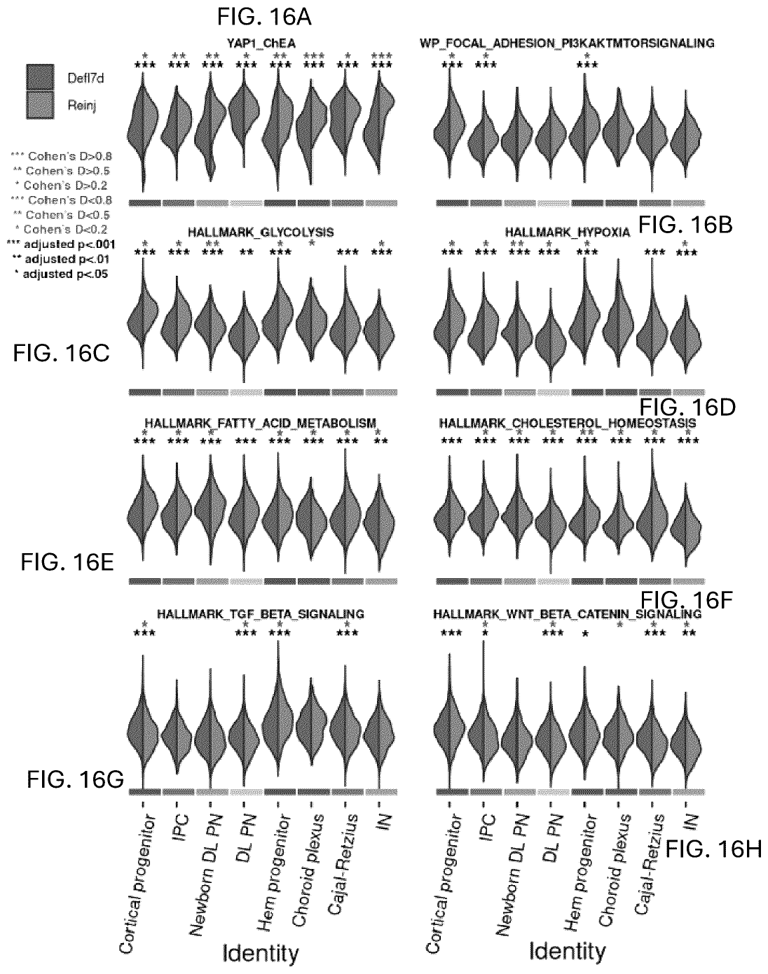

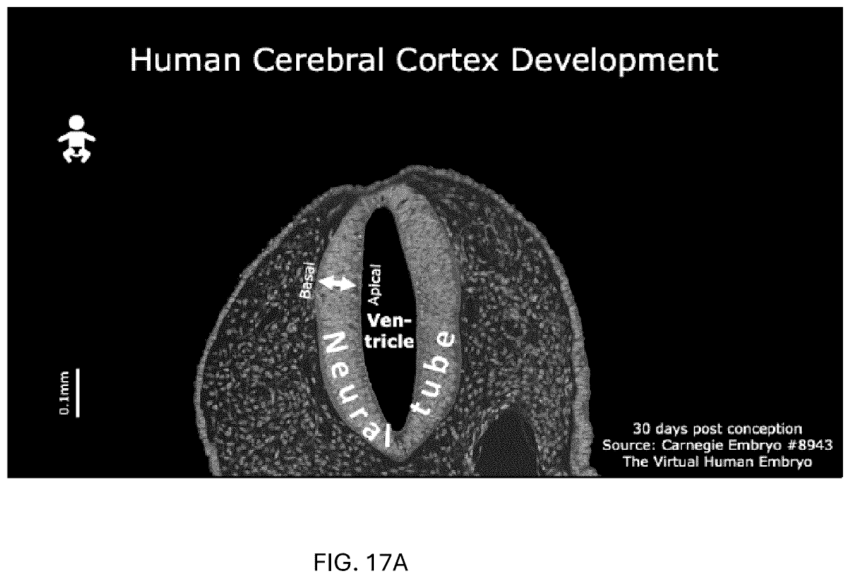

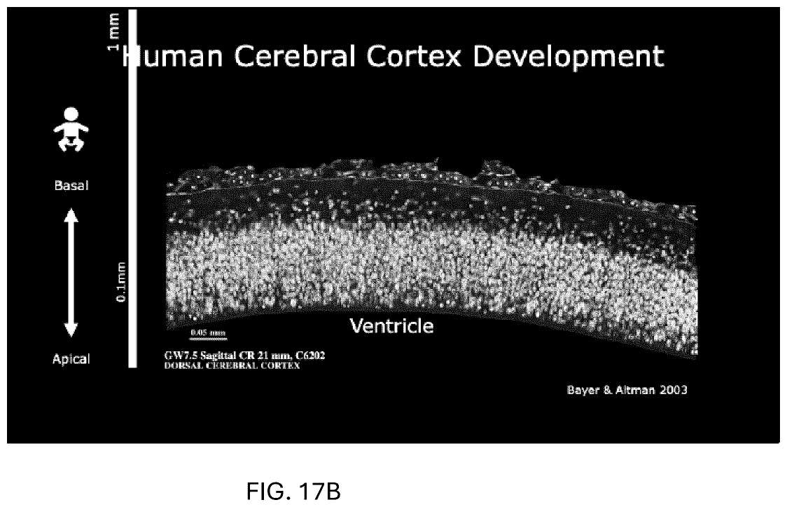

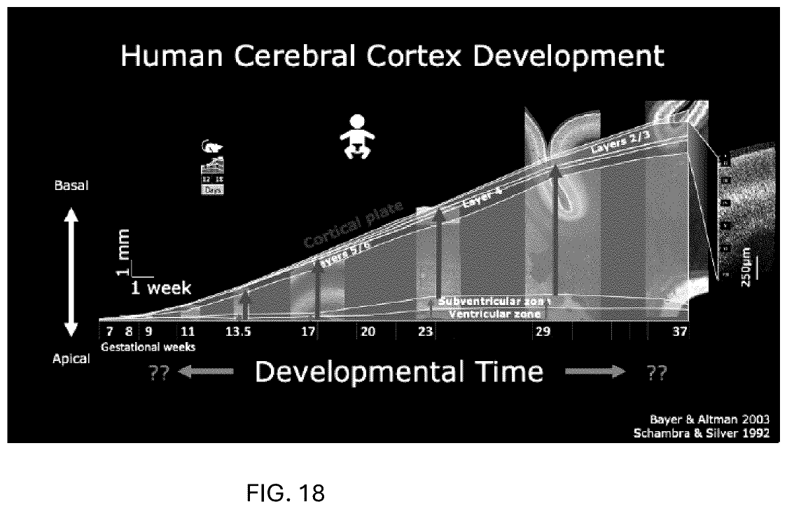

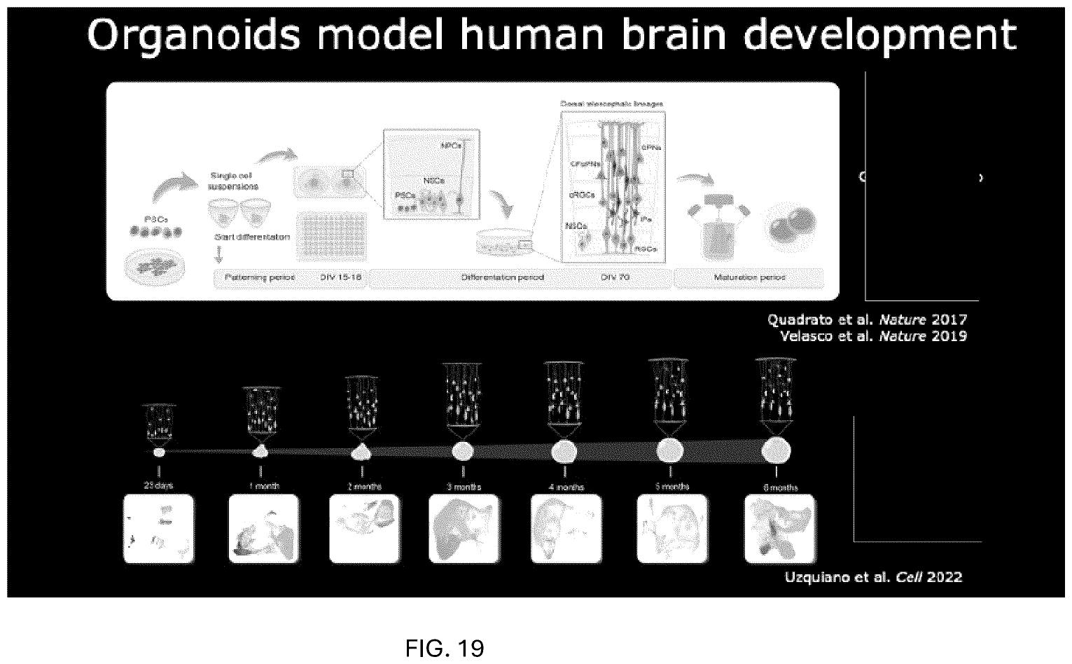

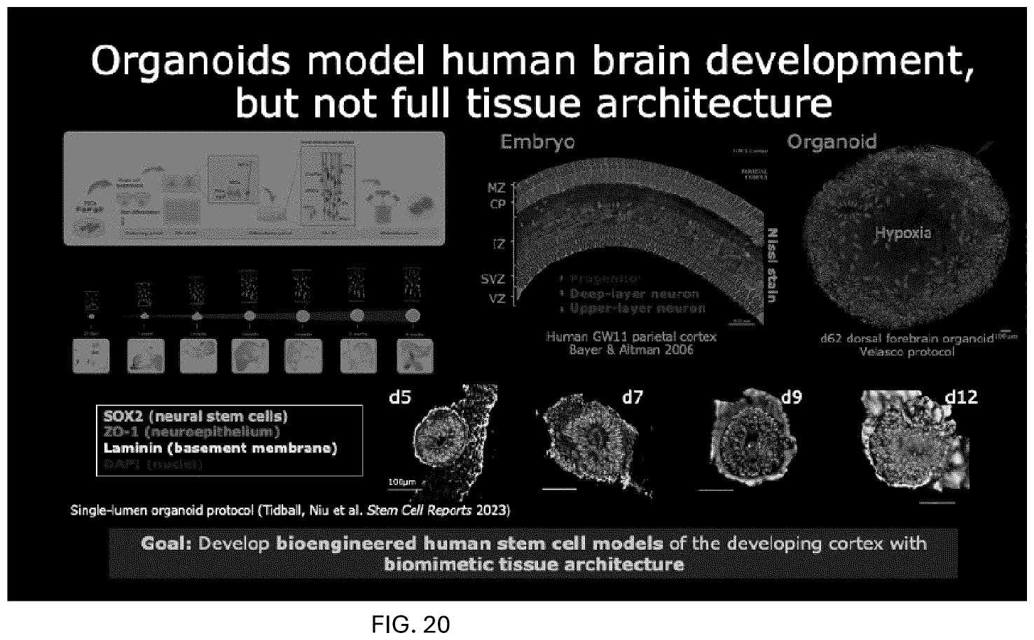

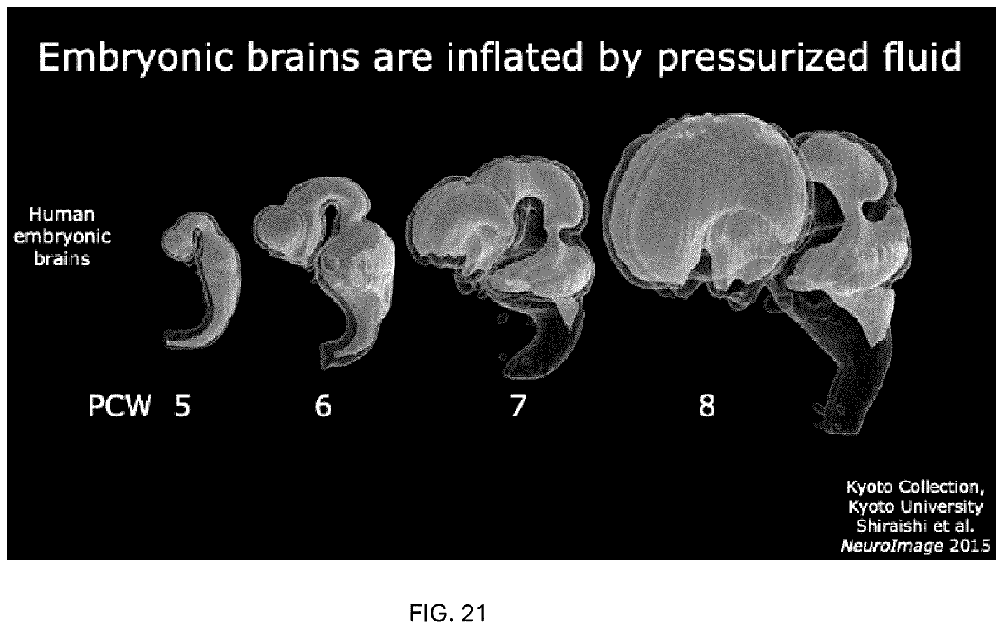

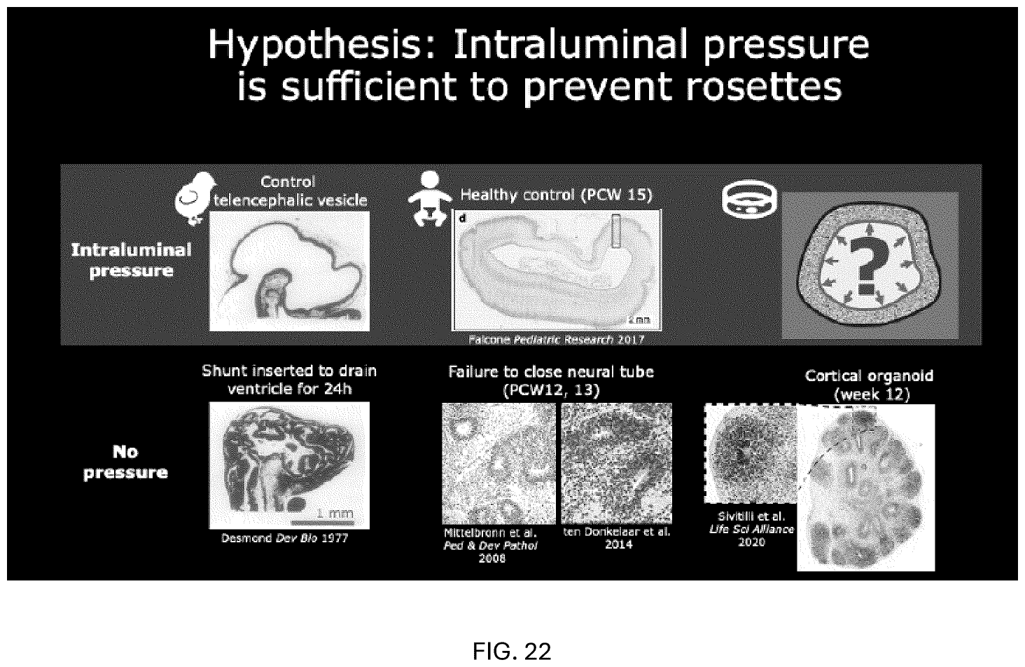

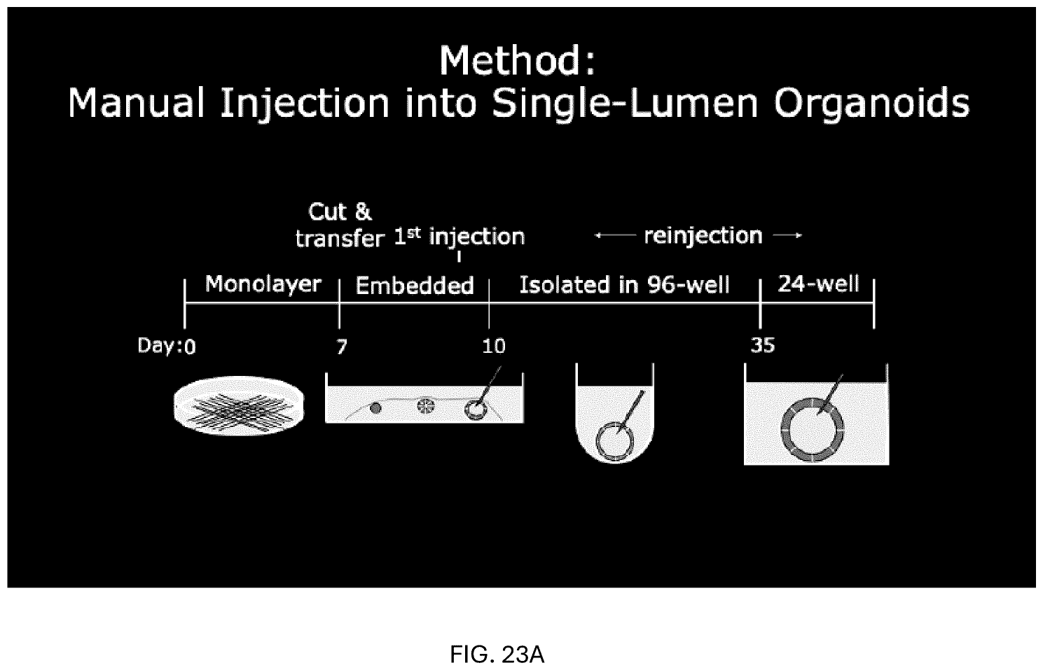

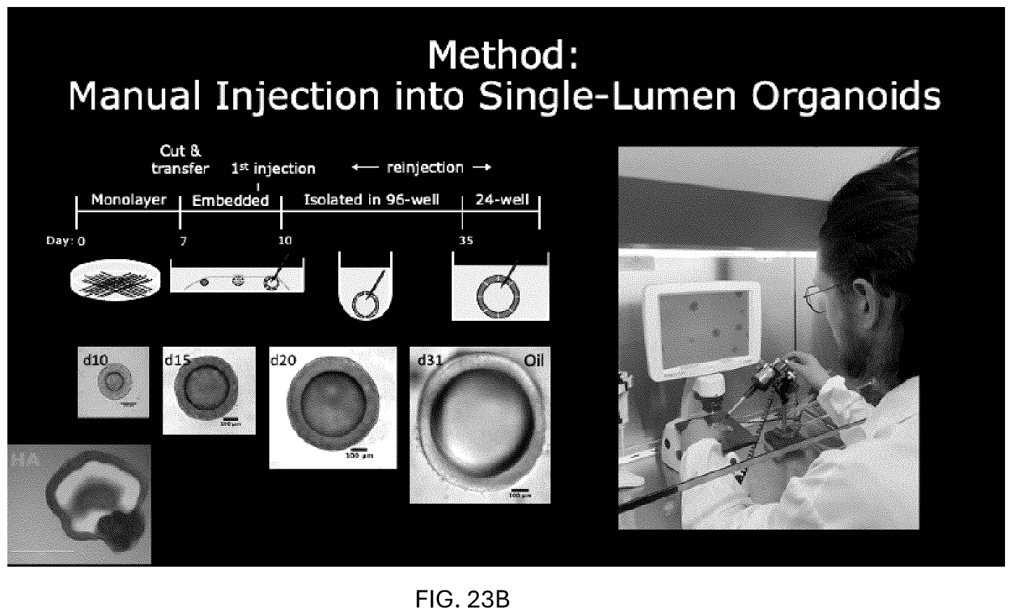

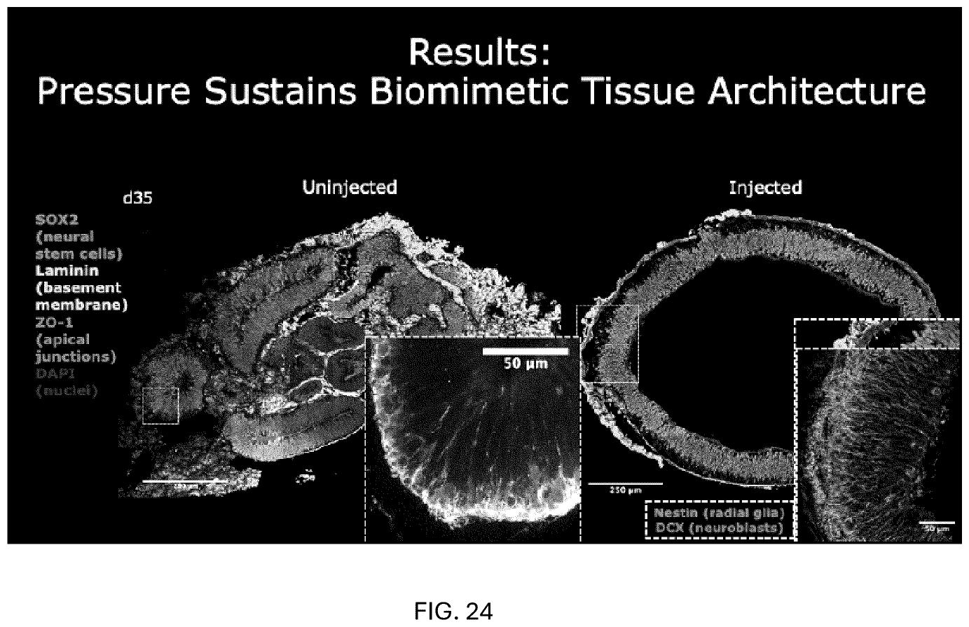

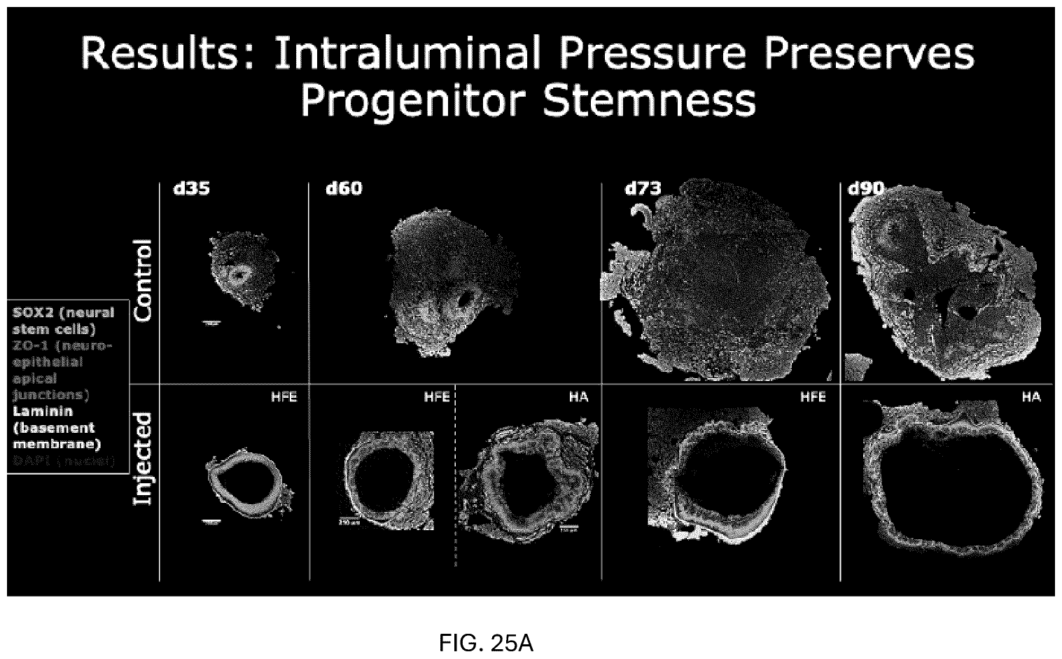

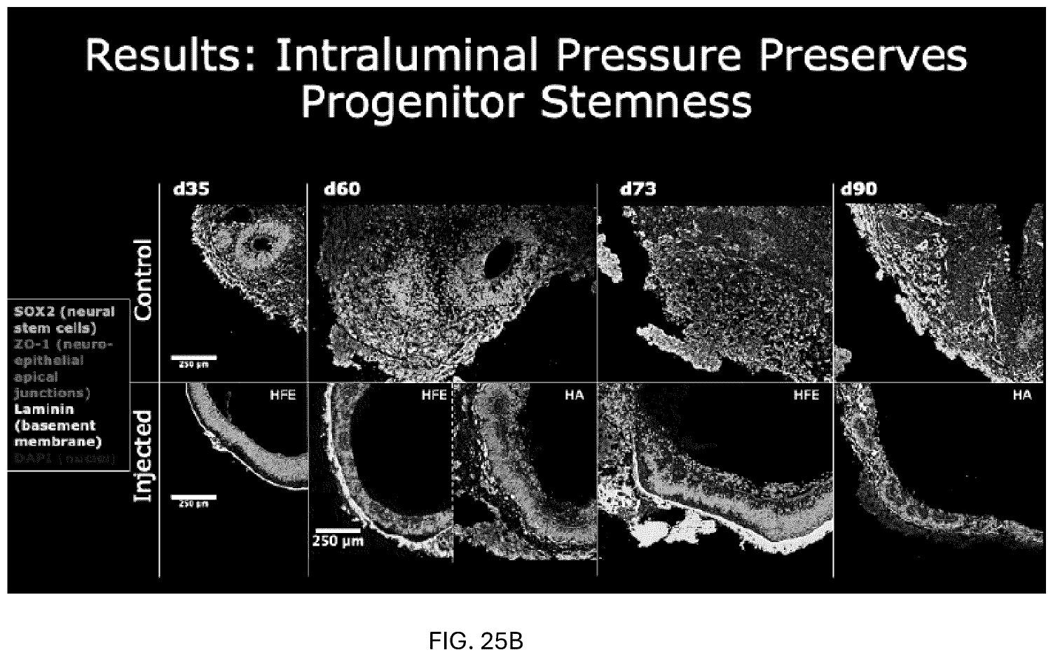

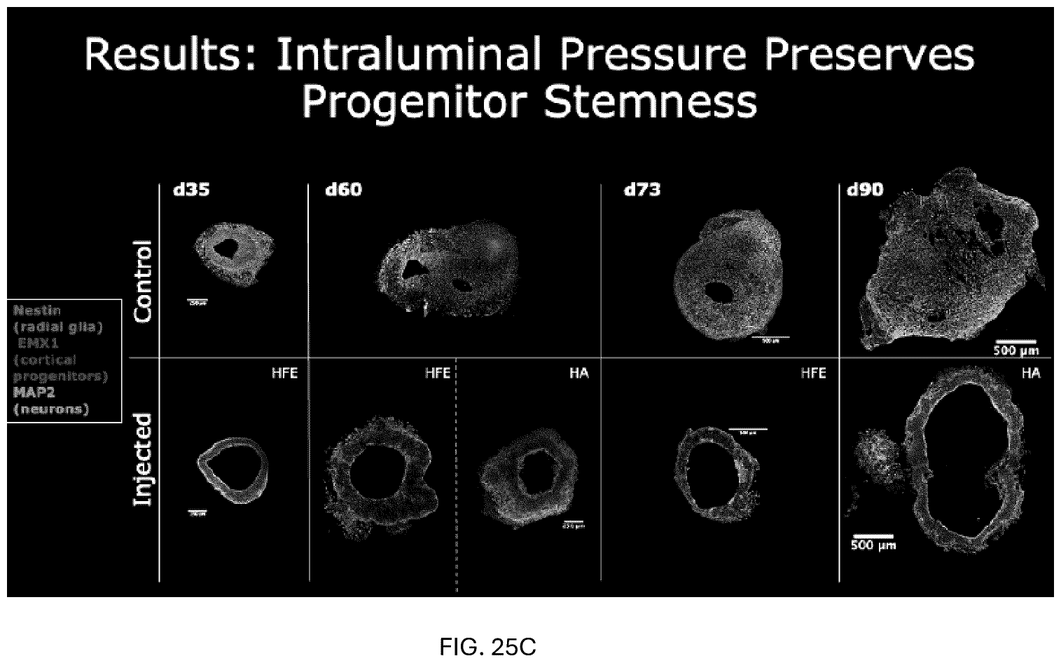

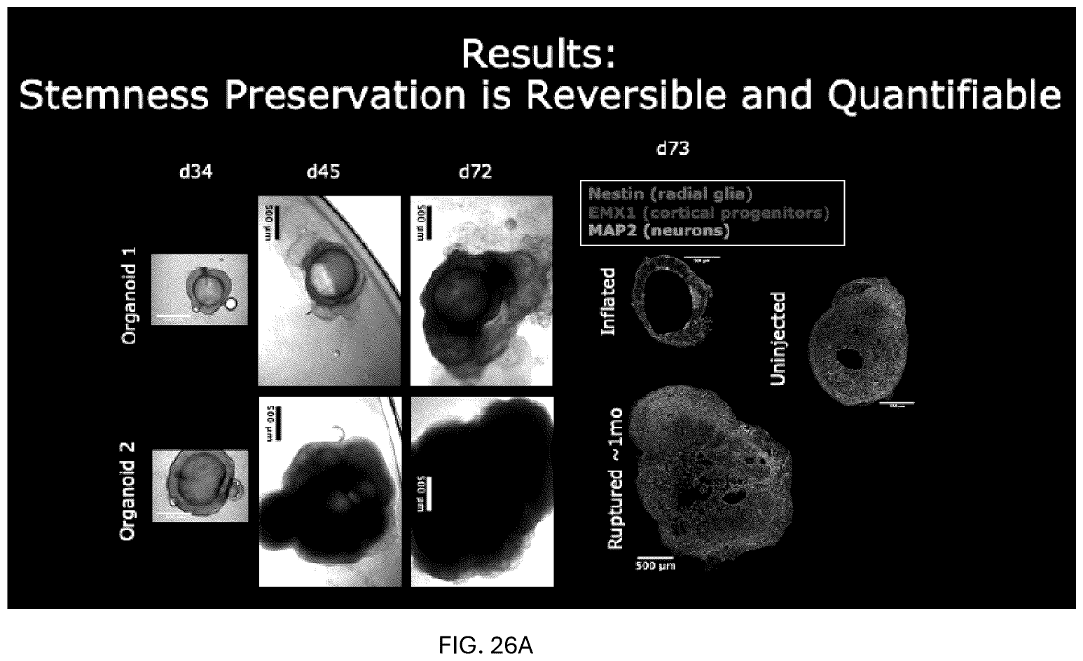

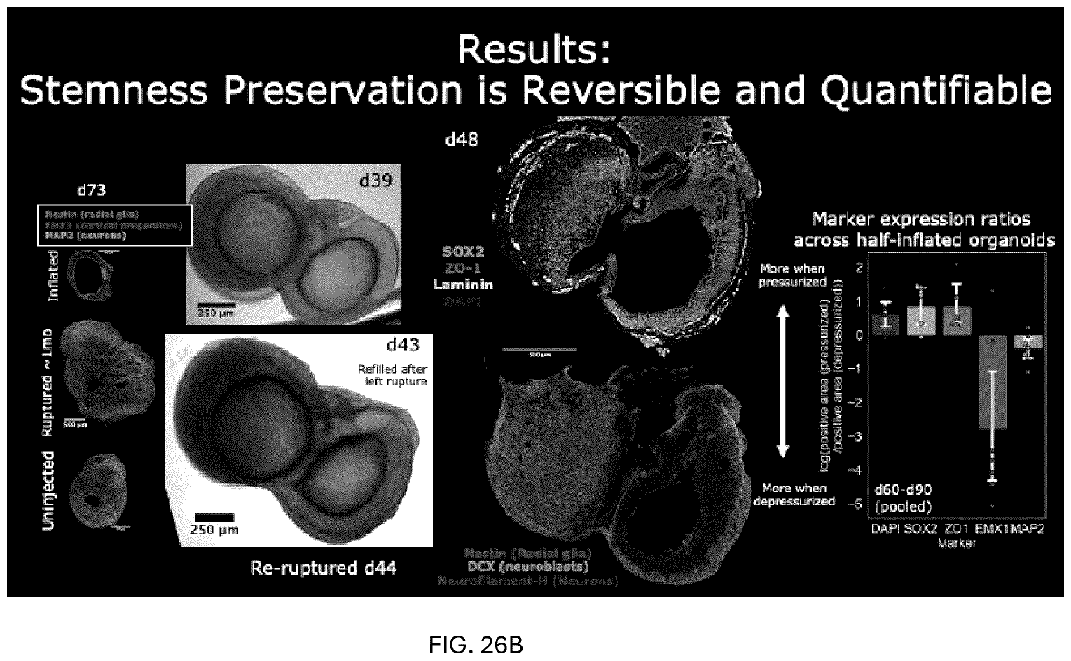

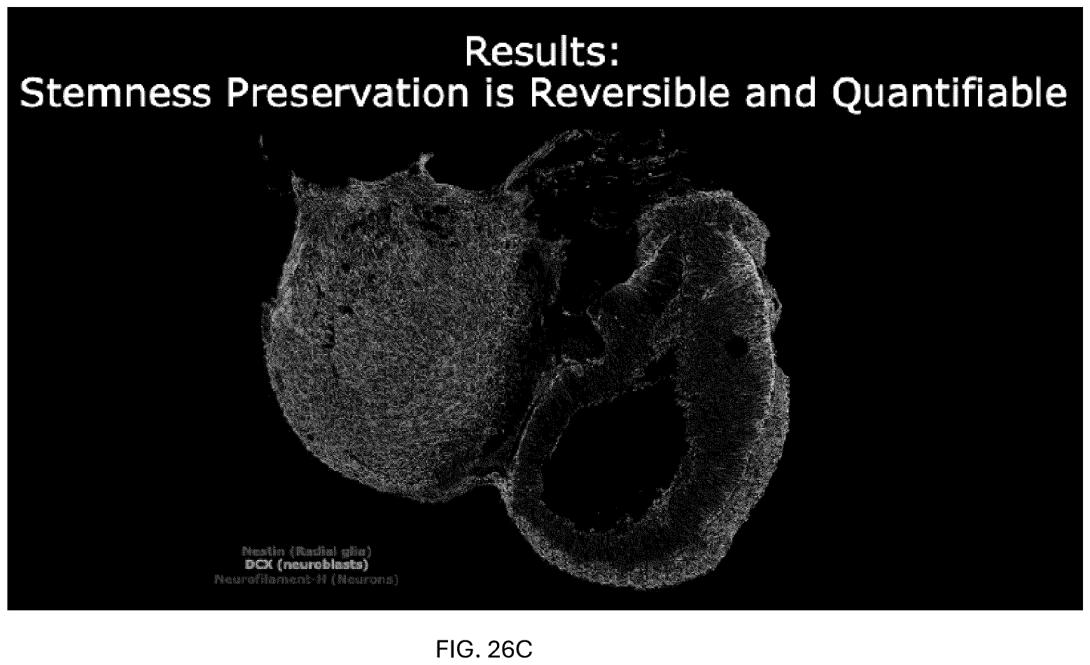

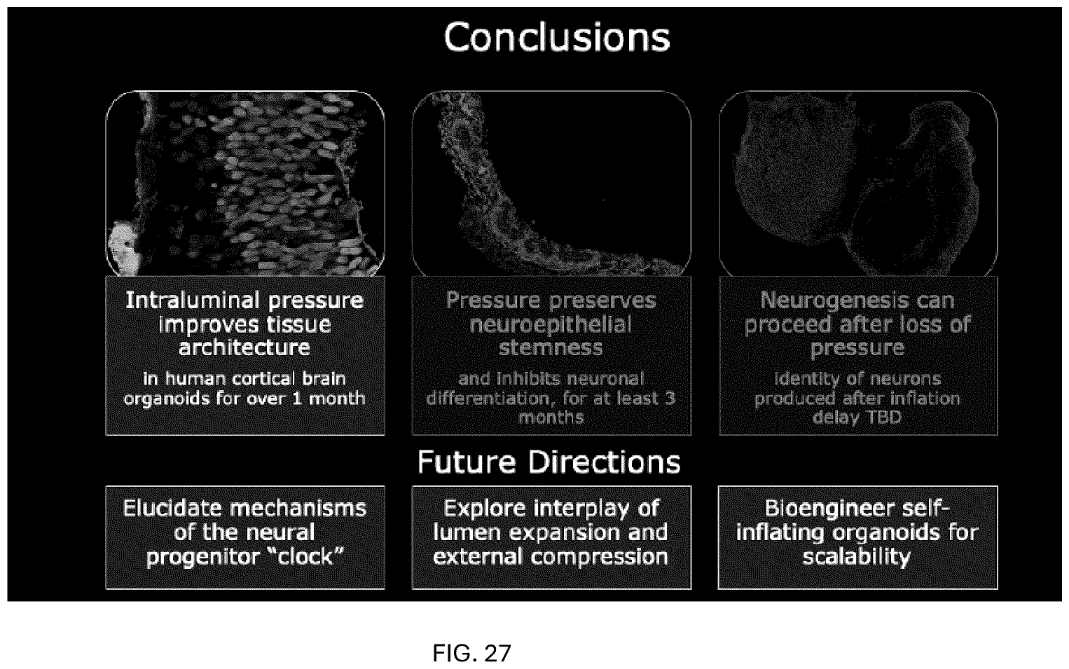

To the inventors knowledge, the only pathway that has specifically been proposed to transduce force from intraluminal pressure into increased mitosis in neuroepithelial tissue is focal adhesion kinase (FAK) signaling. A 2014 paper used immunolabeling and Western blotting to find that the active, phosphorylated form of FAK changed localization from the basal to apical surface of embryonic chick brains exposed to experimental hyperpressure and proposed that this mechanism could explain the increase in mitosis previously observed in hyperpressure experiments (Desmond et al. 2014). This explanation was referenced but not experimentally validated in a later paper that built a computational model of chick brain morphogenesis that included a term for pressure-induced growth (Garcia et al. 2019). Consistent with this proposal, the inventors find that the pathway comprising focal adhesion activation through PI3K and AKT to MAPK signaling is enriched in reinjected compared with deflated organoids, and this increase is disproportionately concentrated in progenitor populations (FIG. 16B).

The redundant Hippo pathway effectors YAP and TAZ have a well-established effect on neurogenic timing during brain development: extensive evidence shows that YAP/TAZ overexpression inhibits neuronal differentiation of NESCs, while YAP/TAZ inhibition leads to premature differentiation and disorganization of apical progenitors as seen in conventional uninjected organoids (Lavado et al. 2021; Terry et al. 2022; Namoto et al. 2024). Notably, the YAP/TAZ signaling pathway is directly activated by mechanical stretch in many epithelial tissues (Aragona et al. 2013). These features make Hippo signaling a logical candidate mechanism for mechanotransduction of intraluminal inflation into the proliferative effects described herein. Consistent with this hypothesis, the inventors found that the YAP_ChEA module score was strongly upregulated across cell types in reinjected compared with deflated organoids (FIG. 16A), although it should be noted that tests using smaller modules of YAP target genes did not show a consistent effect. YAP-targeted genes were even more strongly upregulated in neurons than in progenitors, possibly reflecting these cell's greater elaboration of fibers aligned in the tangential direction and therefore strongly responding to stretch. The functional importance of this signaling, if any, is unclear; in vivo, although YAP is difficult to detect in neurons by immunolabeling, there is growing evidence that it nonetheless plays an active role in regulating processes such as neurite morphogenesis (Terry and Seonhee Kim 2022). In sum, the known functions of YAP/TAZ signaling and support in the single-cell data for its selective activity in inflated organoids make this a leading candidate to explain the effects of inflation on neural stem cell self-renewal and differentiation, worthy of further experimental exploration and validation.

Fatty acid, cholesterol, and lipid biosynthesis-related pathways were frequently identified during gene set enrichment analysis of inflation effects on cortical progenitors in the dataset (FIGS. 16E-16F). Fatty acid metabolism has been shown to be a limiting factor in neural progenitor proliferation; deficiencies in lipid biosynthesis have been shown to result in microcephaly and intellectual disability (Bowers et al. 2020; Namba et al. 2021). Specifically, reduction in cholesterol biosynthesis can disrupt progenitor proliferation and result in premature neurogenesis in a mouse model (Driver et al. 2016). Furthermore, a human-specific mutation that increases fatty acid synthesis in basal radial glia is necessary for the full human-specific expansion of neocortical progenitor and neuron count, especially in the frontal lobes (Pinson et al. 2022). It is possible that the changes the inventors observe are downstream of increased division in inflated organoids, necessitating more rapid membrane production. However, there is also evidence that plasma membrane tension and cytoskeletal mechanisms can directly transduce mechanical signals, such as changes in ECM stiffness, into altered lipid biosynthesis (Romani et al. 2020; Le Roux et al. 2019). Intriguingly, it has recently been shown that the mechanically gated channel Piezo1 promotes neural stem cell expansion and growth in mouse embryos prior to neurogenesis, by maintaining high levels of expression of the cholesterol synthesis pathway (Nourse et al. 2022). However, in the data, only 0.4% of cells had even a single count of PIEZO1 detected, consistent with very low expression of this transcript prior to six months in a recent cortical organoid atlas (Uzquiano et al. 2022). Therefore, it is unlikely that this specific mechanism is responsible for upregulating cholesterol biosynthesis in response to mechanical force. In addition, fatty acid biosynthesis modules were only weakly changed between cell clusters in injected or deflated groups, with Cohen's D between −0.5 and 0.5 for all clusters, suggesting that this process is not likely to be central to the observed phenotype. Nonetheless, the data are consistent with at least partial limitation of symmetric neural stem cell proliferation in non-pressurized organoids by the availability of fatty acids. These results also do not rule out a potential mechanism in which mechanical stretching of neuroepithelia stimulates production of these molecules to facilitate neural progenitor division and prevent premature differentiation.

In the case of Wnt/β-catenin signaling, the slight increases in activity seen in deflated progenitors (FIG. 16) may be due to the release of β-catenin from adherens junctions, as a result of the loss of tangential tension in the neuroepithelium (Campàs, Noordstra, and Yap 2023). However, the small and inconsistent size of this effect indicates that it is unlikely to be a primary driver of the phenotypes described herein. In addition, β-catenin signaling promote neural stemness, while its sequestration promotes cell cycle exit and neurogenesis (Veeraval et al. 2020). Therefore, sequestration of β-catenin at more-active adherens junctions is unlikely to drive the pro-stemness effects of chronic pressure that the inventors observe.

Overall, there are multiple, strong, and non-exclusive candidate mechanisms for converting the mechanical force of tangential stretch into increased neuroepithelial stem cell proliferation. Future directions for investigating possible mechanisms for the molecular transduction of mechanical forces from lumen inflation are further discussed below.

### Potential In Vivo Relevance of Inflation-Mediated Regulation of Neural Differentiation.

The results are consistent with past work showing that pressure is needed to maintain normal histology during ex vivo embryonic development (Desmond and Jacobson 1977; Alonso et al. 1998), and that intraluminal pressure increases mitosis in the short term (Alonso et al. 2000; Desmond et al. 2014). However, the in vivo relevance of the longer-term effect of inflation on the timing of neuronal differentiation remains to be investigated. Intriguingly, in chick, the establishment of a patent aperture between the intraventricular cavity and the subarachnoid space outside the developing brain corresponds in time with to a transient drop in eCSF pressure relative to the amniotic fluid (Jelínek and Pexieder 1970; Alonso et al. 1998; Desmond et al. 2005), and these events immediately precede the onset of forebrain neurogenesis (Tsai et al. 1981). Similarly, dye transport studies in mice (Wang et al. 1997) and humans (Brocklehurst 1969) have shown that communication with the subarachnoid space is established close to the onset of neurogenesis, at approximately E13 in mice and between seven and nine weeks after conception in humans. It has been proposed that this correlation reflects a role for the presence of eCSF in the subarachnoid space in promoting neurogenesis (Gato et al. 2020). The inventors hypothesize that a complementary effect of this opening could be to equalize the pressure inside and outside the cerebral wall, leading to a wave of neurogenesis as was observed in deflated organoids (FIG. 13).

Furthermore, the inventors speculate that variations between species in the timing and extent of intra-luminal pressure during early embryogenesis, driven by variation in the composition of early eCSF (Bueno et al. 2020) or the timing of its release into the subarachnoid space (Rasmussen et al. 2022), may play a previously unappreciated role in determining cortical scaling across evolution. Growing evidence shows that variation in mammalian cortices across species are grounded in alterations in the timing of conserved modular processes such as neural stem cell expansion (Fenlon 2022; Lindhout et al. 2024). This model is thought to facilitate evolvability by minimizing disruption to the complex gene networks that orchestrate the specification of diverse cortical neuroglial cell types, allowing subtle changes in the relative timing of different modules to induce striking diversity in final cortex size and features (Paolino et al. 2023; Senovilla-Ganzo and García-Moreno 2024). However, the mechanisms that control the onset and duration of such processes have been elusive, with candidates identified to date primarily constituting cell-autonomous protein and regulatory variants (Lindhout et al. 2024; Ciceri et al. 2024). The results suggest that the intraluminal pressure present during neural tube expansion could provide such a mechanism, specifically by delaying the transition from symmetric progenitor expansion to neurogenesis across the entire dorsal forebrain. Further research could investigate this possibility through comparative experimental manipulation of neural tube intraluminal pressure across embryonic development in embryos and 3D stem cell models of different species.

Finally, this mechanism may have implications for congenital abnormalities of the brain-ventricle system, including congenital hydrocephalus, a common and often severe condition in which the size of the ventricles is increased at birth (Kundishora et al. 2021; Duy et al. 2022). The etiology of congenital hydrocephalus remains unclear; traditional hypotheses include eCSF overproduction and impaired drainage, although recent proposals have also added the possibility of excessive neuroepithelial compliance (Duy et al. 2022). The inflation paradigm, which can include drainage of fluorescently labeled HA at a steady rate determined by neuroepithelial permeability properties, could be used to explore these hypotheses using patient-derived and isogenic edited cells. By combining physiological relevance to early brain-ventricle interactions with patient-relevant genetics, this approach could advance the investigation of the complex genetics and mechanisms of these conditions (Kundishora et al. 2021).

### Summary.

In summary, the inventors have shown that restoring the positive intraluminal pressure found in the developing embryo improves the self-organization of human dorsal forebrain organoids, yielding the largest and oldest unilocular organoids reported to date. The inventors also demonstrate that artificial intraluminal pressure promotes continued stem cell identity and delays neurogenic differentiation, while experimentally relieving this pressure leads to rapid differentiation. The inventors propose that intraluminal pressure deserves further investigation as a tool for brain organoid bioengineering, a potentially underappreciated link between ventricle contents and neuroepithelial development in embryonic development, and a possible factor in the evolution of the distinctively expanded human cerebral cortex.

Overall, this work represents a significant advance in the ability to model the morphogenesis of the developing human cerebral cortex in organoids. iSLCOs offer a distinctive opportunity to experimentally model the intraluminal pressure present in typical developing human neural tubes, and to test its effects on developmental neurobiology. Future work aiming to model cortical histogenesis in vitro should consider incorporating a source of pressure, as has been reported in in silico modeling (Garcia et al. 2019). More broadly, these results illustrate the utility of long-term direct access to organoid models of brain development, and underline the importance of mechanobiological forces even in soft and delicate developing tissues.

### REFERENCES

Aaku-Saraste, Eeva, Andrea Hellwig, and Wieland Huttner (1996). “Loss of occludin and functional tight junctions, but not ZO-1, during neural tube closure-Remodeling of the neuroepithelium prior to neurogenesis”. In: Developmental Biology 180.2, pp. 664-679.
Abdel Fattah, Abdel Rahman et al. (2021). “Actuation enhances patterning in human neural tube organoids”. In: Nature Communications 12.1, pp. 1-13.
Adesnik, Hillel and Alexander Naka (2018). “Cracking the Function of Layers in the Sensory Cortex”. In: Neuron 100.5, pp. 1028-1043.
Afonso, Cristina and Domingos Henrique (October 2006). “PAR3 acts as a molecular organizer to define the apical domain of chick neuroepithelial cells”. In: Journal of Cell Science 119.20, pp. 4293-4304.
Akther, Fahima et al. (2020). “Hydrogels as artificial matrices for cell seeding in microfluidic devices”. In: RSC Advances 10.71, pp. 43682-43703.
Ali, Shahzad et al. (October 2015). “The effect of Young's modulus on the neuronal differentiation of mouse embryonic stem cells”. In: Acta Biomaterialia 25, pp. 253-267.
Almeida, Glaucia M. et al. (February 2025). “Human brain tissue cultures: a unique ex vivo model to unravel the pathogenesis of neurotropic arboviruses”. In: Current Opinion in Virology 70, p. 101453.
Alonso, M. I. et al. (2000). “Enzymatic digestion of neural tube fluid proteoglycans leads to brain growth disruption”. In: European Journal of Anatomy 4.3, pp. 161-167.
Alonso, Maria Isabel, Angel Gato, J. A. Moro, and E. Barbosa (1998). “Disruption of proteoglycans in neural tube fluid by β-D-xyloside alters brain enlargement in chick embryos”. In: Anatomical Record 252.4, pp. 499-508.
Alonso, Maria Isabel, Angel Gato, J. A. Moro, P. Martin, et al. (1999). “Involvement of sulfated proteoglycans in embryonic brain expansion at earliest stages of development in rat embryos”. In: Cells Tissues Organs 165.1, pp. 1-9.
Amin, Neal D et al. (2023). “Generating human neural diversity with a multi-plexed morphogen screen in organoids”. In: pp. 1-21.
Andrews, Madeline G. et al. (September 2022). “How mechanisms of stem cell polarity shape the human cerebral cortex”. In: Nature Reviews Neuroscience 2022 23:12, pp. 711-724.
Ann, Mary, Megan Cleaton, and Amanda Kirby (2018). “Why Do We Find it so Hard to Calculate the Burden of Neurodevelopmental Disorders?” In: 4.3, p. 10.
Anton-Bolanos, Noelia et al. (June 2024). “Brain Chimeroids reveal individual susceptibility to neurotoxic triggers”. In: Nature 2024 631:8019, pp. 142-149.
Ao, Zheng et al. (2021). “Tubular human brain organoids to model microglia-mediated neuroinflammation”. In: Lab on a Chip.
Applegate, Marissa C. and Dmitriy Aronov (April 2022). “Flexible use of memory by foodcaching birds”. In: eLife 11.
Aragona, Mariaceleste et al. (August 2013). “A mechanical checkpoint controls multicellular growth through YAP/TAZ regulation by actin-processing factors”. In: Cell 154.5, pp. 1047-1059.
Arellano, Jon I. et al. (2021). “Radial Glial Cells: New Views on Old Questions”. In: Neurochemical Research 46.10, pp. 2512-2524.
Autuori, Maria C. et al. (March 2017). “Use of high-frequency ultrasound to study the prenatal development of cranial neural tube defects and hydrocephalus in Gldc-deficient mice”. In: Prenatal Diagnosis 37.3, pp. 273-281.
Axpe, Eneko et al. (July 2020). “Towards brain-tissue-like biomaterials”. In: Nature Communications 2020 11:1, pp. 1-4.
Azab, Waleed et al. (2014). “Blake's pouch cyst”. In: Surgical Neurology International 5. Supplement, p. 112.
Azkona, Garikoitz and Rosario Sanchez-Pernaute (October 2022). “Mice in translational neuroscience: What R we doing?” In: Progress in Neurobiology 217, p. 102330.
Bagley, Joshua A. et al. (2017). “Fused cerebral organoids model interactions between brain regions”. In: Nature Methods 14.7, pp. 743-751.
Bailey, Jordan, Anthony Oliveri, and Edward D. Levin (January 2013). “Zebrafish model systems for developmental neurobehavioral toxicology”. In: Birth Defects Research Part C: Embryo Today: Reviews 99.1, pp. 14-23.
Bak, Aniella et al. (April 2024). “Human organotypic brain slice cultures: a detailed and improved protocol for preparation and long-term maintenance”. In: Journal of Neuroscience Methods 404, p. 110055.
Banerjee, Akhilesh et al. (September 2009). “The influence of hydrogel modulus on the proliferation and differentiation of encapsulated neural stem cells”. In: Biomaterials 30.27, pp. 4695-4699.
Baret, Jean Christophe (January 2012). “Surfactants in droplet-based microfluidics”. In: Lab on a Chip 12.3, pp. 422-433.
Barnes, J. Matthew, Laralynne Przybyla, and Valerie M. Weaver (January 2017). “Tissue mechanics regulate brain development, homeostasis and disease”. In: Journal of Cell Science 130.1, pp. 71-82.
Bayer, Shirley A. and Joseph Altman (2006). The human brain during the late first trimester, pp. 1-592.
Ben-Reuven, Lihi and Orly Reiner (December 2020). “Toward Spatial Identities in Human Brain Organoids-on-Chip Induced by Morphogen-Soaked Beads”. In: Bioengineering 7.4, p. 164.
Bendriem, Raphael M. et al. (2019). “Tight junction protein occludin regulates progenitor self-renewal and survival in developing cortex”. In: eLife 8.
Benito-Kwiecinski, Silvia, Stefano L. Giandomenico, et al. (2021). “An early cell shape transition drives evolutionary expansion of the human forebrain”. In: Cell 184.8, pp. 2084-2102.
Benito-Kwiecinski, Silvia and Madeline Lancaster (2020). “Brain organoids: Human neurodevelopment in a dish”. In: Cold Spring Harbor Perspectives in Biology 12.8, pp. 1-18.
Bergquist, H. (June 1959). “Experiments on the ‘Overgrowth’ Phenomenon in the Brain of Chick Embryos”. In: Development 7.2, pp. 122-127.
Bergquist, Harry (June 1960). “Volumetric Investigations on Overgrowth (Hypermorphosis) in Chick Embryo Brains”. In: Development 8.2, pp. 69-72.
Bethlehem, R. A. I. et al. (April 2022). “Brain charts for the human lifespan”. In: Nature 2022 604:7906 604.7906, pp. 525-533.
Bhaduri, Aparna et al. (2020). “Cell stress in cortical organoids impairs molecular subtype specification”. In: Nature 578.7793, pp. 142-148.
Bhinge, Akshay et al. (June 2014). “MiR-135b is a direct PAX6 target and specifies human neuroectoderm by inhibiting TGF-β/BMP signaling”. In: EMBO Journal 33.11, pp. 1271-1283.
Binninger, E. A., D. O. Schachtschabel, and J. W. Rohen (1987). “Exogenous glycosaminoglycans stimulate hyaluronic acid synthesis by cultured human trabecular-meshwork cells”. In: Experimental Eye Research 45.1, pp. 169-177.
Birch, Jonathan (August 2024). “When is a brain organoid a sentience candidate?” In: Molecular Psychology: Brain, Behavior, and Society 2, p. 22.
Birey, Fikri et al. (2017). “Assembly of functionally integrated human forebrain spheroids”. In: Nature 545.7652, pp. 54-59.
Birnbaum, Rebecca et al. (July 2014). “Prenatal expression patterns of genes associated with neuropsychiatric disorders”. In: American Journal of Psychiatry 171.7, pp. 758-767.
Birtele, Marcella, Madeline Lancaster, and Giorgia Quadrato (December 2024). “Modelling human brain development and disease with organoids”. In: Nature Reviews Molecular Cell Biology 2024, pp. 1-24.
Bowers, Megan et al. (July 2020). “FASN-Dependent Lipid Metabolism Links Neurogenic Stem/Progenitor Cell Activity to Learning and Memory Deficits”. In: Cell Stem Cell 27.1, pp. 98-109.
Braun, Emelie et al. (2023). “Comprehensive cell atlas of the first-trimester developing human brain”. In: Science 382.6667.
Britto, Joanne, David Tannahill, and Roger Keynes (January 2002). “A critical role for sonic hedgehog signaling in the early expansion of the developing brain”. In: Nature Neuroscience 2002 5:2, pp. 103-110.
Brocklehurst, Gordon (1969). “The development of the human cerebrospinal fluid pathway with particular reference to the roof of the fourth ventricle”. In: J. Anat 105.3, pp. 467-475.
Brooks, Eric et al. (February 2025). “A single-cell atlas of spatial and temporal gene expression in the mouse cranial neural plate”. In: eLife 13.
Bubna-Litic, Matyas and Roberto Mayor (June 2025). “Beyond mechanosensing: How cells sense and shape their physical environment during development”. In: Current Opinion in Cell Biology 94, p. 102514.
Bueno, David et al. (2020). “Embryonic cerebrospinal fluid formation and regulation”. In: Seminars in Cell and Developmental Biology 102. September 2019, pp. 3-12.
Cameron, Darren et al. (January 2023). “Single-Nuclei RNA Sequencing of 5 Regions of the Human Prenatal Brain Implicates Developing Neuron Populations in Genetic Risk for Schizophrenia”. In: Biological Psychiatry 93.2, pp. 157-166.
Cammermeyer, Jan (1971). “Median and caudal apertues in the roof of the fourth ventricle in rodents and primates”. In: Journal of Comparative Neurology 141.4, pp. 499-512.
Campas, Otger, Ivar Noordstra, and Alpha S. Yap (December 2023). “Adherens junctions as molecular regulators of emergent tissue mechanics”. In: Nature Reviews Molecular Cell Biology 2023 25:4 25.4, pp. 252-269.
Camps, Camille Cassel De, Saba Aslani, Nicholas Stylianesis, Harris Nami, Thomas M Durcan, et al. (n.d.). “Supporting Information”. In: ( ) pp. 2-5.
Camps, Camille Cassel De, Saba Aslani, Nicholas Stylianesis, Harris Nami, Nguyen-vi Mohamed, et al. (2022). “Hydrogel Mechanics Influence the Growth and Development of Embedded Brain Organoids”.
Cao, Xinwei, Samuel L. Pfaff, and Fred H. Gage (December 2008). “YAP regulates neural progenitor cell number via the TEA domain transcription factor”. In: Genes & Development 22.23, pp. 3320-3334.
Caporale, Nicolo et al. (December 2024). “Multiplexing cortical brain organoids for the longitudinal dissection of developmental traits at single-cell resolution”. In: Nature Methods 2024 22:2, pp. 358-370.
Capps, Benjamin (October 2023). “What Do Chimeras Think About?” In: Cambridge Quarterly of Healthcare Ethics 32.4, pp. 496-514.
Catala, Martin (2005). “Development of the Cerebrospinal Fluid Pathways During Embryonic and Fetal Life in Humans”. In: Pediatric Hydrocephalus. Ed. by G.
Cinalli, C. Sainte-Rose, and W. J. Maixner. Springer, Milano, pp. 19-45. isbn: 978-88-470-2121-1.
Cavanaugh, Jr, J. Oswari, and S. S. Margulies (December 2012). “Role of Stretch on Tight Junction Structure in Alveolar Epithelial Cells”.
Cederquist, Gustav Y. et al. (2019). “Specification of positional identity in forebrain organoids”. In: Nature Biotechnology 37.4, pp. 436-444.
Cevik Tufan, A et al. (2004). “Shell-less culture of the chick embryo as a model system in the study of developmental neurobiology”. In: 3, pp. 8-11.
Chambers, Stuart M. et al. (2009). “Highly efficient neural conversion of human ES and iPS cells by dual inhibition of SMAD signaling”. In: Nature Biotechnology 27.3, p. 485.
Chang, Jessica T., Laura Anne Lowery, and Hazel Sive (2012). “Multiple roles for the Na,KATPase subunits, Atp1a1 and Fxyd1, during brain ventricle development”. In: Developmental Biology 368.2, pp. 312-322.
Chau, Kevin et al. (2015). “Progressive Differentiation and Instructive Capacities of Amniotic Fluid and Cerebrospinal Fluid Proteomes following Neural Tube Closure”. In: Developmental Cell 35.6, pp. 789-802.
Cheffer, Arquimedes, Attila Tarnok, and Henning Ulrich (December 2013). “Cell cycle regulation during neurogenesis in the embryonic and adult brain”. In: Stem Cell Reviews and Reports 9.6, pp. 794-805.
Chen, L., P. T. Russell, and W. J. Larsen (1993). “Functional significance of cumulus expansion in the mouse: Roles for the preovulatory synthesis of hyaluronic acid within the cumulus mass”. In: Molecular Reproduction and Development 34.1, pp. 87-93.
Chen, Lin et al. (1990). “Hyaluronic acid synthesis and gap junction endocytosis are necessary for normal expansion of the cumulus mass”. In: Molecular Reproduction and Development 26.3, pp. 236-247.
Chen, Yu et al. (2021). A versatile polypharmacology platform promotes cytoprotection and viability of human pluripotent and differentiated cells. Vol. 18. 5. Springer US, pp. 528-541.
Chenn, Anjen and Christopher A. Walsh (July 2002). “Regulation of cerebral cortical size by control of cell cycle exit in neural precursors”. In: Science 297.5580, pp. 365-369.
Cherry, Jocie F. et al. (2014). “Engineered N-cadherin and L1 biomimetic substrates concertedly promote neuronal differentiation, neurite extension and neuroprotection of human neural stem cells”. In: Acta Biomaterialia 10.10, pp. 4113-4126.
Cheung, Justin P., Justin D. Tubbs, and Pak C. Sham (August 2022). “Extended gene set analysis of human neuro-psychiatric traits shows enrichment in brain-expressed human accelerated regions across development”. In: Schizophrenia Research 246, pp. 148-155.
Chiaradia, Ilaria et al. (2023). “Tissue morphology influences the temporal program of human brain organoid development”. In: Cell Stem Cell 30.10, pp. 1351-1367.
Choe, Youngshik and Samuel J. Pleasure (2018). “Meningeal Bmps Regulate Cortical Layer Formation”. In: Brain Plasticity 4.2, pp. 169-183.

Chong, Brian W. et al. (October 1997). “A magnetic resonance template for normal cerebellar development in the human fetus”. In: Neurosurgery 41.4, pp. 924-929.
Chu, Xin Yi, Yuan Quan, and Hong Yu Zhang (May 2020). “Human accelerated genome regions with value in medical genetics and drug discovery”. In: Drug Discovery Today 25.5, pp. 821-827.
Chung S-H; Cooke, J (1978). “Observations on the formation of the brain and of nerve connections following embryonic manipulation of the amphibian neural tube”. In: Proceedings of the Royal Society B 201, p. 3733.
Ciceri, Gabriele et al. (January 2024). “An epigenetic barrier sets the timing of human neuronal maturation”. In: Nature 2024 626:8000, pp. 881-890.
Como, Christina N., Sol Kim, and Julie Siegenthaler (2023). “Stuck on you: Meninges cellular crosstalk in development”. In: Current Opinion in Neurobiology 79, p. 102676.
Coquand, Laure et al. (2024). “A cell fate decision map reveals abundant direct neurogenesis bypassing intermediate progenitors in the human developing neocortex”. In: Nature Cell Biology 26.5, pp. 698-709.
Correa, Gustavo Gumz, Lazaro Faria Amaral, and Leonardo Modesti Vedolin (December 2011). “Neuroimaging of Dandy-Walker malformation: New concepts”. In: Topics in Magnetic Resonance Imaging 22.6, pp. 303-312.
Coulombre, Alfred J. and Jane L. Coulombre (1958). “The role of mechanical factors in brain morphogenesis”. In: American Association of Anatomists Seventy-First Annual Session. Buffalo, NY.
Courtney, Ya'el et al. (January 2024). “A choroid plexus apocrine secretion mechanism shapes CSF proteome and embryonic brain development”. In: bioRxiv, p. 2024.01.08.574486.
Crawford, T. Quinn and Henk Roelink (March 2007). “The notch response inhibitor DAPT enhances neuronal differentiation in embryonic stem cell-derived embryoid bodies independently of sonic hedgehog signaling”. In: Developmental dynamics: an official publication of the American Association of Anatomists 236.3, pp. 886-892.
Curzio, Domenico L. Di and Domenico L. Di Curzio (December 2017). “Animal Models of Hydrocephalus”. In: Open Journal of Modern Neurosurgery 8.1, pp. 57-71.
Damianidou, Eleni, Lidia Mouratidou, and Christina Kyrousi (October 2022). “Research models of neurodevelopmental disorders: The right model in the right place”. In: Frontiers in Neuroscience 16, p. 1031075.
Desmond, Mary (June 1985). “Reduced number of brain cells in so-called neural overgrowth”. In: The Anatomical Record 212.2, pp. 195-198.
Desmond, Mary and Antone G. Jacobson (1977). “Embryonic brain enlargement requires cerebrospinal fluid pressure”. In: Developmental Biology 57.1, pp. 188-198.
Desmond, Mary, Janice Knepper, et al. (2014). “Focal adhesion kinase as a mechanotransducer during rapid brain growth of the chick embryo”. In: International Journal of Developmental Biology 58.1, pp. 35-43.
Desmond, Mary, Michael Levitan, and Andrew Haas (2005). “Internal luminal pressure during early chick embryonic brain growth: Descriptive and empirical observations”. In: Anatomical Record-Part A Discoveries in Molecular, Cellular, and Evolutionary Biology 285.2, pp. 737-747.
DeSpenza, Tyrone, Marina Carlson, et al. (2021). “PTEN mutations in autism spectrum disorder and congenital hydrocephalus: developmental pleiotropy and therapeutic targets”. In: Trends in Neurosciences 44.12, pp. 961-976.
DeSpenza, Tyrone, Emre Kiziltug, et al. (2023). “Dual impact of PTEN mutation on CSF dynamics and cortical networks via the dysregulation of neural precursors and their interneuron descendants”. In: bioRxiv.
Di Bella, Daniela et al. (2020). “Molecular Logic of Cellular Diversification in the Mammalian Cerebral Cortex”.
Di Bella, Daniela J. et al. (July 2024). “Making Ramon y Cajal proud: Development of cell identity and diversity in the cerebral cortex”. In: Neuron 112.13, pp. 2091-2111.
Dicke, Ursula and Gerhard Roth (January 2016). “Neuronal factors determining high intelligence”. In: Philosophical Transactions of the Royal Society B: Biological Sciences 371.1685.
Doerr, Sophia, Phillip Zhou, and Katerina Ragkousi (2024). “Origin and development of primary animal epithelia”. In: BioEssays 46.2, pp. 1-24.
Donkelaar, Hans J ten et al. (2014). “Neurulation and Neural Tube Defects”. In: Clinical Neuroembryology: Development and Developmental Disorders of the Human Central Nervous System. Berlin, Heidelberg: Springer Berlin Heidelberg, pp. 165-217.
Dou, Jinxin et al. (2020). “Combination Stiffness Gradient with Chemical Stimulation Directs Glioma Cell Migration on a Microfluidic Chip”. In: Analytical Chemistry 92.1, pp. 892-898.
Douceau, Sara, Tanya Deutsch Guerrero, and Julien Ferent (January 2023). “Establishing Hedgehog Gradients during Neural Development”. In: Cells 2023, Vol. 12, Page 225 12.2, p. 225.
Driver, Ashley M. et al. (July 2016). “Altered cholesterol biosynthesis causes precocious neurogenesis in the developing mouse forebrain”. In: Neurobiology of Disease 91, pp. 69-82.
Dupont, Sirio et al. (June 2011). “Role of YAP/TAZ in mechanotransduction”. In: Nature 2011 474:7350, pp. 179-183.
Dutta, Anirban et al. (2022). “Model-based investigation of elasticity and spectral exponent from atomic force microscopy and electrophysiology in normal versus Schizophrenia human cerebral organoids”. In: Proceedings of the Annual International Conference of the IEEE Engineering in Medicine and Biology Society, EMBS 2022-July, pp. 1585-1589.
Duy, Phan Q et al. (2022). “A neural stem cell paradigm of pediatric hydrocephalus”. In: Cerebral Cortex, pp. 1-18.
Edgar, John M., Meghan Robinson, and Stephanie M. Willerth (2017). “Fibrin hydrogels induce mixed dorsal/ventral spinal neuron identities during differentiation of human induced pluripotent stem cells”. In: Acta Biomaterialia 51, pp. 237-245.
Edison, Robin and Maximilian Muenke (March 2003). “The interplay of genetic and environmental factors in craniofacial morphogenesis: holoprosencephaly and the role of cholesterol”. In: Congenital Anomalies 43.1, pp. 1-21.
Eiraku, Mototsugu et al. (2011). “Self-organizing optic-cup morphogenesis in three-dimensional culture”. In: Nature 472.7341, pp. 51-58.
Elias, Laura, Doris Wang, and Arnold Kriegstein (August 2007). “Gap junction adhesion is necessary for radial migration in the neocortex”. In: Nature 2007 448:7156, pp. 901-907.
Ellis, Pam et al. (August 2004). “SOX2, a Persistent Marker for Multipotential Neural Stem Cells Derived from Embryonic Stem Cells, the Embryo or the Adult”. In: Developmental Neuroscience 26.2-4, pp. 148-165.
Elorriaga, Vicente, Alessandra Pierani, and Frederic Causeret (April 2023). “Cajal-retzius cells: Recent advances in identity and function”. In: Current Opinion in Neurobiology 79, p. 102686.
Eroshenko, Nikolai et al. (March 2013). “Effect of substrate stiffness on early human embryonic stem cell differentiation”. In: Journal of Biological Engineering 7.1, pp. 1-8.
Essen, David C. van (December 2020). “A 2020 view of tension-based cortical morphogenesis”. In: Proceedings of the National Academy of Sciences of the United States of America 117.52, pp. 32868-32879.
Eze, Ugomma C. et al. (2021). “Single-cell atlas of early human brain development highlights heterogeneity of human neuroepithelial cells and early radial glia”. In: Nature Neuroscience 24.4, pp. 584-594.
Factor, Phillip et al. (1998). “Overexpression of the Na+,K+-ATPase a1 subunit increases Na+, K+-ATPase function in A549 cells”. In: American Journal of Respiratory Cell and Molecular Biology 18.6, pp. 741-749.
Fame, Ryann, Christian Cortes-Campos, and Hazel Sive (2020). “Brain Ventricular System and Cerebrospinal Fluid Development and Function: Light at the End of the Tube: A Primer with Latest Insights”. In: BioEssays 42.3, pp. 1-15.
Fame, Ryann and Maria Lehtinen (2020). “Emergence and Developmental Roles of the Cerebrospinal Fluid System”. In: Developmental Cell 52.3, pp. 261-275.
Fan, Pan et al. (2022). “The Application of Brain Organoids in Assessing Neural Toxicity”. In: Frontiers in Molecular Neuroscience 15. February, pp. 1-11.
Fedorchak, Nikolai, Nisha Iyer, and Randolph Ashton (2020). “Bioengineering tissue morphogenesis and function in human neural organoids”. In: Seminars in Cell and Developmental Biology 111. April, pp. 52-59.
Fedorova, Veronika et al. (2019). “Differentiation of neural rosettes from human pluripotent stem cells in vitro is sequentially regulated on a molecular level and accomplished by the mechanism reminiscent of secondary neurulation”. In: Stem Cell Research 40. August, p. 101563.
Fenlon, Laura R. (2022). “Timing as a Mechanism of Development and Evolution in the Cerebral Cortex”. In: Brain, Behavior and Evolution 97.2, pp. 8-32.
Ferent, Julien, Donia Zaidi, and Fiona Francis (2020). “Extracellular Control of Radial Glia Proliferation and Scaffolding During Cortical Development and Pathology”. In: Frontiers in Cell and Developmental Biology 8. October.
Fietz, Simone A. et al. (July 2012). “Transcriptomes of germinal zones of human and mouse fetal neocortex suggest a role of extracellular matrix in progenitor self-renewal”. In: Proceedings of the National Academy of Sciences of the United States of America 109.29, pp. 11836-11841.
Finger, Stanley (January 2005). “Galen: The Birth of Experimentation”. In: Minds Behind the Brain. Oxford University Press, pp. 39-52.
Finnell, Richard H. et al. (May 2021). “Gene Environment Interactions in the Etiology of Neural Tube Defects”. In: Frontiers in Genetics 12, p. 659612.
Florio, Marta and Wieland Huttner (June 2014). “Neural progenitors, neurogenesis and the evolution of the neocortex”. In: Development 141.11, pp. 2182-2194.
Franklin, J. Matthew, Zhengming Wu, and Kun Liang Guan (June 2023). “Insights into recent findings and clinical application of YAP and TAZ in cancer”. In: Nature Reviews Cancer 2023 23:8, pp. 512-525.
Friede, Reinhard L. (1989). “Gross and Microscopic Development of the Central Nervous System”. In: Developmental Neuropathology. Springer, Berlin, Heidelberg, pp. 2-20.
Frowein, Julia von, Andrea Wizenmann, and Magdalena Gotz (August 2006). “The transcription factors Emx1 and Emx2 suppress choroid plexus development and promote neuroepithelial cell fate”. In: Developmental Biology 296.1, pp. 239-252.
Gandhi, Jarel K. et al. (2019). “Human Fibrinogen for Maintenance and Differentiation of Induced Pluripotent Stem Cells in Two Dimensions and Three Dimensions”. In: Stem Cells Translational Medicine 8.6, pp. 512-521.
Garcia, Kara et al. (2019). “Molecular and mechanical signals determine morphogenesis of the cerebral hemispheres in the chicken embryo”.
Garreta, Elena et al. (2021). “Rethinking organoid technology through bioengineering”. In: Nature Materials 20.2, pp. 145-155.
Gato, Angel, Maria Isabel Alonso, et al. (2020). “Neurogenesis: A process ontogenically linked to brain cavities and their content, CSF”. In: Seminars in Cell and Developmental Biology 102. October 2019, pp. 21-27.
Gato, Angel and Mary Desmond (2009). “Why the embryo still matters: CSF and the neuroepithelium as interdependent regulators of embryonic brain growth, morphogenesis and histiogenesis”. In: Developmental Biology 327.2, pp. 263-272.
Gertz, Caitlyn and Arnold Kriegstein (October 2015). “Neuronal Migration Dynamics in the Developing Ferret Cortex”. In: Journal of Neuroscience 35.42, pp. 14307-14315.
Ghiselli, Giancarlo and Marco Maccarana (2016). “Drugs affecting glycosaminoglycan metabolism”. In: Drug Discovery Today 21.7, pp. 1162-1169.
Giandomenico, Stefano L. et al. (2019). “Cerebral organoids at the air-liquid interface generate diverse nerve tracts with functional output”. In: Nature Neuroscience 22.4, pp. 669-679.
Gordon, Aaron et al. (February 2021). “Long-term maturation of human cortical organoids matches key early postnatal transitions”. In: Nature Neuroscience 2021 24:3, pp. 331-342.
Gross, Charles (1998). “Brain, vision, memory: tales in the history of neuroscience”. In: MIT Press. Chap. The rebirth of brain science.
Grove, Elizabeth (2023). “Neocortical Neurogenesis in Development and Evolution”. In: ed. By Wieland Huttner. John Wiley & Sons. Chap. Temporal Scaling: A Link between Conserved Neurogenic Processes and Differential Size in Mammalian Cortical Development.
Grydeland, Hakon et al. (March 2019). “Waves of Maturation and Senescence in Micro-structural MRI Markers of Human Cortical Myelination over the Lifespan”. In: Cerebral Cortex 29.3, pp. 1369-1381.
Guo, Chuanyu et al. (May 2018). “Apical Cell-Cell Adhesions Reconcile Symmetry and Asymmetry in Zebrafish Neurulation”. In: iScience 3, pp. 63-85.
Gutzman, Jennifer H. et al. (2008). “Formation of the zebrafish midbrain-hindbrain boundary constriction requires laminin-dependent basal constriction”. In: Mechanisms of Development 125.11-12, pp. 974-983.
Hanashima, Carina and Kenichi Toma (August 2015). “Switching modes in corticogenesis: Mechanisms of neuronal subtype transitions and integration in the cerebral cortex”. In: Frontiers in Neuroscience 9. July, p. 152683.
Hansen, David V. et al. (March 2010). “Neurogenic radial glia in the outer subventricular zone of human neocortex”. In: Nature 2010 464:7288 464.7288, pp. 554-561.
Hao, Yuhan et al. (June 2021). “Integrated analysis of multimodal single-cell data”. In: Cell 184.13, pp. 3573-3587.
Harding, Katherine and Kristin White (October 2018). “Drosophila as a Model for Developmental Biology: Stem Cell-Fate Decisions in the Developing Nervous System”. In: Journal of Developmental Biology 2018, Vol. 6, Page 25 6.4, p. 25.
Harris, James, Giulio Srubek Tomassy, and Paola Arlotta (2015). “Building blocks of the cerebral cortex: From development to the dish”. In: Wiley Interdisciplinary Reviews: Developmental Biology 4.5, pp. 529-544.
He, Wenteng et al. (June 2024). “Divergent roles of SOX2 in human and mouse germ cell specification related to X-linked gene dosage effects”. In: bioRxiv, p. 2024.06.25.599839.
He, Zhisong, Leander Dony, Jonas Simon Fleck, Artur Szałata, Katelyn X. Li, Irena Sliškovi'c, Hsiu-Chuan Lin, Malgorzata Santel, Alexander Atamian, Giorgia Quadrato, Jieran Sun, Sergiu Pasca, Neal D. Amin, et al. (November 2024). “An integrated transcriptomic cell atlas of human neural organoids”. In: Nature 2024 635:8039 635.8039, pp. 690-698.
He, Zhisong, Leander Dony, Jonas Simon Fleck, Artur Szałata, Katelyn X. Li, Irena Sliškovi'c, Hsiu-Chuan Lin, Malgorzata Santel, Alexander Atamian, Giorgia Quadrato, Jieran Sun, Sergiu Pasca, J. Gray Camp, et al. (2023). “An integrated transcriptomic cell atlas of humanneural organoids”. In: bioRxiv, p. 2023.10.05.561097.
Hebert, Jean M et al. (2011). “FGFs: Neurodevelopment's Jack-of-all-Trades—How Do They Do it?” In: Frontiers in Neuroscience 5, December, p. 133.
Hendriks, Delilah et al. (February 2024). “Human fetal brain self-organizes into long-term expanding organoids”. In: Cell 187.3, pp. 712-732.
Hirose, Tomonori et al. (April 2006). “PAR3 is essential for cyst-mediated epicardial development by establishing apical cortical domains”. In: Development 133.7, pp. 1389-1398.
Homan, Kimberly A. et al. (2016). “Bioprinting of 3D Convoluted Renal Proximal Tubules on Perfusable Chips”. In: Scientific Reports 6, pp. 1-13.
Hori, A. et al. (1984). “Exencephaly in Cantrell-Haller-Ravitsch syndrome”. In: Acta Neuropathologica 65.2, pp. 158-162.
H'ribkova, Hana et al. (2018). “Five steps to form neural rosettes: structure and function”. In: Journal of Cell Science January.
Huang, Xi et al. (August 2009). “Sonic hedgehog signaling regulates a novel epithelial progenitor domain of the hindbrain choroid plexus”. In: Development 136.15, pp. 2535-2543.
Hulme, Amy J. et al. (2022). “Making neurons, made easy: The use of Neurogenin-2 in neuronal differentiation”. In: Stem Cell Reports 17.1, pp. 14-34.
Humpel, C. (October 2015). “Organotypic brain slice cultures: A review”. In: Neuroscience 305, pp. 86-98.
Huttner, Wieland, ed. (2023). Neocortical Neurogenesis in Development and Evolution. John Wiley & Sons. isbn: 9781119860914.
Hyun, Insoo et al. (2024). “The Brainstorm Organoid Project: A Collaboration of Bioethics and Neuroscience to Advance Brain Organoid Research”. In: pp. 49-56. Iacopetti, Paola et al. (April 1999). “Expression of the antiproliferative gene TIS21 at the onset of neurogenesis identifies single neuroepithelial cells that switch from proliferative to neuron-generating division”. In: Proceedings of the National Academy of Sciences of the United States of America 96.8, pp. 4639-4644.
Inoue, Takashi et al. (2008). “Zic deficiency in the cortical marginal zone and meninges results in cortical lamination defects resembling those in type II lissencephaly”. In: Journal of Neuroscience 28.18, pp. 4712-4725.
Ishihara, Keisuke et al. (2017). “Reconstitution of a patterned neural tube from single mouse embryonic stem cells”. In: Methods in Molecular Biology 1597. March, pp. 43-55.
Iskusnykh, Igor Y. et al. (September 2023). “Lmx1a is a master regulator of the cortical hem”. In: eLife 12.
Jalilian, Elmira and Su Ryon Shin (2023). “Novel model of cortical-meningeal organoid coculture system improves human cortical brain organoid cytoarchitecture”. In: Scientific Reports 13.1, pp. 1-12.
Jang, Ahram and Maria Lehtinen (May 2022). “Experimental approaches for manipulating choroid plexus epithelial cells”. In: Fluids and Barriers of the CNS 2022 19:1, pp. 1-15.
Javier-Torrent, Miriam and Laurent Nguyen (2023). “Cortical Neuron Migration in Health and Disease”. In: ed. by Wieland Huttner. John Wiley & Sons. Chap. Dynamic Transcriptional Control of Neural Stem Cells.
Javier-Torrent, Miriam, Geraldine Zimmer-Bensch, and Laurent Nguyen (February 2021). “Mechanical Forces Orchestrate Brain Development”. In: Trends in Neurosciences 44.2, pp. 110-121.
Jelinek, R. (1961). “A report to the so called “overgrowth” of the neural tube”. In: C ̌eskoslovenska Morfologie 9.2, pp. 151-161.
Jelinek, R. and T. Pexieder (1970). “Pressure of the CSF and the morphogenesis of the CNS. I. Chick embryo.” In: Folia Morphologica 18.2, pp. 102-110.
Johansson, Pia A, Helen B Stolp, and Joao C Sousa (2014). “The choroid plexuses and their impact on developmental neurogenesis”. In: Frontiers in Neuroscience 8, October, p. 340.
Johansson, Pia A. et al. (March 2013). “The transcription factor Otx2 regulates choroid plexus development and function”. In: Development 140.5, pp. 1055-1066. issn: 0950-1991.
Joutsa, Juho, Daniel T. Corp, and Michael D. Fox (August 2022). “Lesion network mapping for symptom localization: Recent developments and future directions”. In: Current Opinion in Neurology 35.4, pp. 453-459.
Kadoshima, Taisuke et al. (2013). “Self-organization of axial polarity, inside-out layer pattern, and species-specific progenitor dynamics in human ES cell-derived neocortex”. In: Proceedings of the National Academy of Sciences of the United States of America 110.50, pp. 20284-20289.
Kadowaki, Masakazu et al. (April 2007). “N-cadherin mediates cortical organization in the mouse brain”. In: Developmental Biology 304.1, pp. 22-33.
Kageyama, Ryoichiro and Hiromi Shimojo (2023). “Neocortical Neurogenesis in Development and Evolution”. In: ed. by Wieland Huttner. John Wiley & Sons. Chap. Dynamic Transcriptional Control of Neural Stem Cells.
Kalebic, Nereo et al. (April 2019). “Neocortical Expansion Due to Increased Proliferation of Basal Progenitors Is Linked to Changes in Their Morphology”. In: Cell Stem Cell 24.4, pp. 535-550.
Kallen, B. (January 1965). “Proliferation in the Embryonic Brain with Special Reference to the Overgrowth Phenomenon and its Possible Relationship to Neoplasia”. In: Progress in Brain Research 14.C, pp. 263-278.
Kancherla, Vijaya (July 2023). “Neural tube defects: a review of global prevalence, causes, and primary prevention”. In: Child's Nervous System 39.7, pp. 1703-1710.
Kanton, Sabina and Sergiu Pasca (October 2022). “Human assembloids”. In: Development (Cambridge) 149.23.
Karzbrun, Eyal, Aimal H. Khankhel, et al. (2021). “Human neural tube morphogenesis in vitro by geometric constraints”. In: Nature 599.7884, pp. 268-272.
Karzbrun, Eyal, Aditya Kshirsagar, et al. (2018). “Human brain organoids on a chip reveal the physics of folding”. In: Nature Physics 14.5, pp. 515-522.
Kawasaki, Hiroshi (April 2024). “Investigation of the mechanisms underlying the development and evolution of folds of the cerebrum using gyrencephalic ferrets”. In: Journal of Comparative Neurology 532.4, e25615.
Keller, Ray, Lance A. Davidson, and David R. Shook (April 2003). “How we are shaped: The biomechanics of gastrulation”. In: Differentiation 71.3, pp. 171-205.
Kelley, Kevin W. et al. (July 2024). “Host circuit engagement of human cortical organoids transplanted in rodents”. In: Nature Protocols 2024 19:12 19.12, pp. 3542-3567.
Kittock, Claire M. and Louis Jan Pilaz (April 2023). “Advances in in utero electroporation”. In: Developmental Neurobiology 83.3-4, pp. 73-90.
Klika, E. and R. Jelinek (1961). “Histogenesis of the Experimental Exencephalia in the Chick”. In: C′ eskoslovenska Morfologie 9.2, pp. 162-172.
Klingler, Esther, Sergi Roig Puiggros, and Denis Jabaudon (2023). “Neocortical Neurogenesis in Development and Evolution”. In: ed. by Wieland Huttner. John Wiley & Sons. Chap. Intrinsic and Input-dependent Development of Cortical Neuron Types.
Knight, Gavin T. et al. (2018). “Engineering induction of singular neural rosette emergence within hPSC-derived tissues”. In: eLife 7, pp. 1-23.
Ko, Jihoon et al. (April 2024). “Microfluidic high-throughput 3D cell culture”. In: Nature Reviews Bioengineering 2024 2:6, pp. 453-469.
Kolodziejczyk, Annika et al. (2024). “Electroporation of Sliced Human Cortical Organoids for Studies of Gene Function.” In: Journal of visualized experiments: JoVE 213, pp. 1-22.
Kompanikova, Petra and Vit ̆ezslav Bryja (May 2022). “Regulation of choroid plexus development and its functions”. In: Cellular and Molecular Life Sciences 2022 79:6 79.6, pp. 1-15.
Koo, Bonsang et al. (June 2023). “Setting the clock of neural progenitor cells during mammalian corticogenesis”. In: Seminars in Cell & Developmental Biology 142, pp. 43-53.
Kostic, Milos et al. (April 2019). “YAP Activity Is Necessary and Sufficient for Basal Progenitor Abundance and Proliferation in the Developing Neocortex”. In: Cell Reports 27.4, pp. 1103-1118.
Kratochvil, Michael J. et al. (2019). “Engineered materials for organoid systems”. In: Nature Reviews Materials 4.9, pp. 606-622.
Krzeczkowski, John E. and Ryan J. Van Lieshout (April 2019). “Prenatal influences on the development and stability of personality”. In: New Ideas in Psychology 53, pp. 22-31.
Kundishora, Adam J. et al. (November 2021). “Genomics of human congenital hydrocephalus”. In: Child's Nervous System 37.11, pp. 3325-3340.
Labibi, Bita et al. (September 2021). “High-content imaging and analysis to quantify the nuclear to cytoplasmic ratio of TGFβ and hippo effectors in mammalian cells”. In: STAR Protocols 2.3, p. 100632.
Lacey, Tiara, Ryann Fame, and Maria Lehtinen (2023). “Neocortical Neurogenesis in Development and Evolution”. In: ed. by Wieland Huttner. John Wiley & Sons. Chap. Keeping the Cortex Afloat: Cerebrospinal Fluid Contributions to Cerebral Cortical Development.
Lachmann, Alexander et al. (August 2010). “ChEA: transcription factor regulation inferred from integrating genome-wide ChIP-X experiments”. In: Bioinformatics (Oxford, England) 26.19, pp. 2438-2444.
Lampersperger, Hanna et al. (January 2025). “Mechanical impact on neural stem cell lineage decisions in human brain organoids”. In: bioRxiv, p. 2025.01.08.631895.
Lancaster, Madeline (October 2024). “Unraveling mechanisms of human brain evolution”. In: Cell 187.21, pp. 5838-5857.
Lancaster, Madeline, Nina S. Corsini, et al. (2017). “Guided self-organization and cortical plate formation in human brain organoids”. In: Nature Biotechnology 35.7, pp. 659-666.
Lancaster, Madeline and Juergen Knoblich (2012). “Spindle orientation in mammalian cerebral cortical development”. In: Current Opinion in Neurobiology 22.5, pp. 737-746.
Lancaster, Madeline, Magdalena Renner, et al. (2013). “Cerebral organoids model human brain development and microcephaly”. In: Nature 501.7467, pp. 373-379.
Lavado, Alfonso, Ruchika Gangwar, et al. (2021). “YAP/TAZ maintain the proliferative capacity and structural organization of radial glial cells during brain development”. In: Developmental Biology 480. August, pp. 39-49.
Lavado, Alfonso, Jun Young Park, et al. (2018). “The Hippo Pathway Prevents YAP/TAZ-Driven Hypertranscription and Controls Neural Progenitor Number”. In: Developmental Cell 47.5, pp. 576-591.
Le Roux, Anabel Lise et al. (August 2019). “The plasma membrane as a mechanochemical transducer”. In: Philosophical Transactions of the Royal Society B 374.1779.
Lee, Chun Ting, Raphael M. Bendriem, et al. (August 2017). “3D brain Organoids derived from pluripotent stem cells: promising experimental models for brain development and neurodegenerative disorders”. In: Journal of Biomedical Science 2017 24:1, pp. 1-12.
Lee, Chun Ting, Jia Chen, et al. (2017). “CYP3A5 Mediates Effects of Cocaine on Human Neocorticogenesis: Studies using an in Vitro 3D Self-Organized hPSC Model with a Single Cortex-Like Unit”. In: Neuropsychopharmacology 42.3, pp. 774-784.
Lee, Sangmoon and Joseph G. Gleeson (July 2020). “Closing in on Mechanisms of Open Neural Tube Defects”. In: Trends in Neurosciences 43.7, pp. 519-532.
Lehtinen, Maria et al. (2011). “The Cerebrospinal Fluid Provides a Proliferative Niche for Neural Progenitor Cells”. In: Neuron 69.5, pp. 893-905.
Leifsdottir, Kristin et al. (July 2022). “The cerebrospinal fluid proteome of preterm infants predicts neurodevelopmental outcome”. In: Frontiers in Pediatrics 10, p. 921444.
Leventer, Richard J., Renzo Guerrini, and William B. Dobyns (March 2008). “Malformations of cortical development and epilepsy”. In: Dialogues in Clinical Neuroscience 10.1, pp. 47-19362.
Levi-Montalcini, R. (March 1982). “Developmental neurobiology and the natural history of nerve growth factor.” In: Annual review of neuroscience 5. Volume 5, 1982, pp. 341-362.
Levic, Daniel S. and Michel Bagnat (2023). “Polarized transport of membrane and secreted proteins during lumen morphogenesis”. In: Seminars in Cell and Developmental Biology 133. March 2022, pp. 65-73.
Levine, Ariel J. and Ali H. Brivanlou (August 2007). “Proposal of a model of mammalian neural induction”. In: Developmental Biology 308.2, pp. 247-256.
Lewis, Jonathan and Soren Holm (February 2024). Regulating organoid and organoid-related activities: Proposals to address regulatory gaps and areas of over-regulation. HYBRIDA.
Lewitus, Eric et al. (April 2013). “An Adaptive Threshold in Mammalian Neocortical Evolution”. In: PLOS Biology 12.11, e1002000.
Li, Xiaoquan et al. (June 2023). “Polarized focal adhesion kinase activity within a focal adhesion during cell migration”. In: Nature Chemical Biology 2023 19:12 19.12, pp. 1458-1468.
Li, Xue et al. (July 2023). “Inhibition of Foxp4 Disrupts Cadherin-based Adhesion of Radial Glial Cells, Leading to Abnormal Differentiation and Migration of Cortical Neurons in Mice”. In: Neuroscience Bulletin 39.7, pp. 1131-1145.
Liberzon, Arthur, Chet Birger, et al. (December 2015). “The Molecular Signatures Database (MSigDB) hallmark gene set collection”. In: Cell systems 1.6, p. 417.
Liberzon, Arthur, Aravind Subramanian, et al. (June 2011). “Molecular signatures database (MSigDB) 3.0”. In: Bioinformatics 27.12, pp. 1739-1740.
Liddelow, Shane A. et al. (October 2010). “Development of the lateral ventricular choroid plexus in a marsupial, Monodelphis domestica”. In: Cerebrospinal Fluid Research 7.1, pp. 1-10.
Limbrick, David D. et al. (February 2017). “Cerebrospinal fluid biomarkers of infantile congenital hydrocephalus”. In: PLOS ONE 12.2, e0172353.
Lin, Neil Y. C. et al. (2019). “Renal reabsorption in 3D vascularized proximal tubule models”. In: Proceedings of the National Academy of Sciences of the United States of America 116.12, pp. 5399-5404.
Lindhout, Feline W. et al. (June 2024). “A molecular and cellular perspective on human brain evolution and tempo”. In: Nature 2024 630:8017 630.8017, pp. 596-608.
Liu, Rui et al. (2015). “Fst11 is involved in the regulation of radial glial scaffold development”. In: Molecular Brain 8.1, pp. 1-14.
Llinares-Benadero, Cristina and Victor Borrell (January 2019). “Deconstructing cortical folding: genetic, cellular and mechanical determinants”. In: Nature Reviews Neuroscience 2018 20:3, pp. 161-176.
Lodato, Simona and Paola Arlotta (2015). “Generating Neuronal Diversity in the Mammalian Cerebral Cortex”. In: Annual Review of Cell and Developmental Biology 31, pp. 699-720.
Long, K. et al. (February 2016). “Integrin signalling regulates the expansion of neuroepithelial progenitors and neurogenesis via Wnt7a and Decorin”. In: Nature Communications 2016 7:1, pp. 1-14.
Long, Katherine R. and Wieland Huttner (January 2022). “The Role of the Extracellular Matrixin Neural Progenitor Cell Proliferation and Cortical Folding During Human Neocortex Development”. In: Frontiers in Cellular Neuroscience 15, p. 804649.
Lopatina, Olga L. et al. (December 2019). “Excitation/inhibition imbalance and impaired neurogenesisin neurodevelopmental and neurodegenerative disorders”. In: Reviews in the Neurosciences 30.8, pp. 807-820.
Lowery, Laura Anne and Hazel Sive (2004). “Strategies of vertebrate neurulation and a reevaluation of teleost neural tube formation”. In: Mechanisms of Development 121.10, pp. 1189-1197.
(2005). “Initial formation of zebrafish brain ventricles occurs independently of circulation and requires the nagie oko and snakehead/atplala.1 gene products”. In: Development 132.9, pp. 2057-2067.
Lu, Yang and Haiying Sun (December 2020). “Progress in the Development of Small Molecular Inhibitors of Focal Adhesion Kinase (FAK)”. In: Journal of Medicinal Chemistry 63.23, pp. 14382-14403.
Machado, Lucas Simoes et al. (December 2020). “Generation of neural progenitor cells from porcine-induced pluripotent stem cells”. In: Journal of Tissue Engineering and Regenerative Medicine 14.12, pp. 1880-1891.
Marin, Faustino and Luis Puelles (1994). “Patterning of the embryonic avian midbrain after experimental inversions: A polarizing activity from the isthmus”. In: Developmental Biology 163.1, pp. 19-37.
Marshall, Grant F., Alfredo Gonzalez-Sulser, and Catherine M. Abbott (March 2021). “Modelling epilepsy in the mouse: Challenges and solutions”. In: DMM Disease Models and Mechanisms 14.3.
Martin, Adam C. and Bob Goldstein (May 2014). “Apical constriction: themes and variations on a cellular mechanism driving morphogenesis”. In: Development 141.10, pp. 1987-1998.
Martins-Costa, Catarina et al. (October 2023). “Morphogenesis and development of human telencephalic organoids in the absence and presence of exogenous extracellular matrix”. In: The EMBO Journal 42.22.
Mattingly, Zoe and Sundari Chetty (March 2025). “Untangling the Molecular Mechanisms Contributing to Autism Spectrum Disorder Using Stem Cells”. In: Autism Research 18.3, pp. 476-485.
Mcallister, James P (2012). “Pathophysiology of congenital and neonatal hydrocephalus”. In: Seminars in Fetal and Neonatal Medicine 17, pp. 285-294.
McNulty, Jason D. et al. (2019). “Micro-injection molded, poly(vinyl alcohol)-calcium salt templates for precise customization of 3D hydrogel internal architecture”. In: Acta Biomaterialia 95, pp. 258-268.
Merino, Florencia and Magdalena Gotz (2023). “Neural Stem Cells as Glia”. In: ed. by Wieland Huttner. John Wiley & Sons. Chap. Diversity and Evolution of Human Cortical Progenitor Cell Types.
Merlin, Sam and Trichur Vidyasagar (May 2023). “Optogenetics in primate cortical networks”. In: Frontiers in Neuroanatomy 17, p. 1193949.
Miotto, Mattia et al. (2023). “Collective behavior and self-organization in neural rosette morphogenesis”. In: Frontiers in Cell and Developmental Biology 11. August, pp. 1-18.
Mittelbronn, Michel et al. (2008). “Ependymoblastomatous exencephaly: A unique fetal malformation”. In: Pediatric and Developmental Pathology 11.5, pp. 397-401.
Miura, Yuki et al. (October 2024). “Assembloid model to study loop circuits of the human nervous system”. In: bioRxiv, p. 2024.10.13.617729.
Miyata, Takaki (2023). “Cortical Neuron Migration in Health and Disease”. In: ed. by Wieland Huttner. John Wiley & Sons. Chap. Mechanical and Physical Interactions Involving Neocortical Progenitor Cells.
Miyata, Takaki et al. (January 2015). “Interkinetic nuclear migration generates and opposes ventricular-zone crowding: Insight into tissue mechanics”. In: Frontiers in Cellular Neuroscience 8, January, p. 122988.
Mogensen, Jesper (2021). “Handbook of Laboratory Animal Science”. In: APA. Chap. Animal models in neuroscience, pp. 591-614.
Mole, Matteo A. et al. (February 2020). “Integrin-Mediated Focal Anchorage Drives Epithelial Zippering during Mouse Neural Tube Closure”. In: Developmental Cell 52.3, pp. 321-334.
Molnar, Zoltan et al. (February 2006). “Comparative aspects of cerebral cortical development”. In: European Journal of Neuroscience 23.4, pp. 921-934.
Molyneaux, Bradley J. et al. (2007). “Neuronal subtype specification in the cerebral cortex”. In: Nature Reviews Neuroscience 8.6, pp. 427-437.
Moon, Lauren D. and Fengzhu Xiong (2022). “Mechanics of neural tube morphogenesis”. In: Seminars in Cell and Developmental Biology 130.xxxx, pp. 56-69.
Moore, Samantha A. and Angelo Iulianella (May 2021). “Development of the mammalian cortical hem and its derivatives: The choroid plexus, Cajal-Retzius cells and hippocampus”. In: Open Biology 11.5.
Mora-Berm Udez, Felipe et al. (March 2022). “From stem and progenitor cells to neurons in the developing neocortex: key differences among hominids”. In: The FEBS Journal 289.6, pp. 1524-1535.
Mora-Bermudez, Felipe et al. (July 2022). “Longer metaphase and fewer chromosome segregation errors in modern human than Neanderthal brain development”. In: Science Advances 8.30.
Mostajo-Radji, Mohammed et al. (February 2020). “Reverse engineering human brain evolution using organoid models”. In: Brain Research 1729, p. 146582.
Mulder, Lance A. et al. (2023). “A beginner's guide on the use of brain organoids for neuroscientists: a systematic review”. In: Stem cell research & therapy 14.1, p. 87.
Muniz, Ayse J. et al. (July 2023). “Engineered extracellular matrices facilitate brain organoids from human pluripotent stem cells”. In: Annals of Clinical and Translational Neurology 10.7, pp. 1239-1253.
Nagasaka, Arata et al. (November 2016). “Differences in the mechanical properties of the developing cerebral cortical proliferative zone between mice and ferrets at both the tissue and single-cell levels”. In: Frontiers in Cell and Developmental Biology 4, November, p. 233654.
Namba, Takashi and Wieland Huttner (January 2017). “Neural progenitor cells and their role in the development and evolutionary expansion of the neocortex”. In: Wiley Interdisciplinary Reviews: Developmental Biology 6.1, e256.
(October 2024). “What Makes Us Human: Insights from the Evolution and Development of the Human Neocortex”. In: Annual review of cell and developmental biology 40.1, pp. 427-452.
Namba, Takashi, Jeannette Nardelli, et al. (February 2021). “Metabolic Regulation of Neocortical Expansion in Development and Evolution”. In: Neuron 109.3, pp. 408-419.
Namoto, Kenji et al. (2024). “NIBR-LTSi is a selective LATS kinase inhibitor activating YAP signaling and expanding tissue stem cells in vitro and in vivo”. In: Cell Stem Cell 31.4, pp. 554-569.
Nano, Patricia R. and Aparna Bhaduri (July 2022). “Evaluation of advances in cortical development using model systems”. In: Developmental Neurobiology 82.5, pp. 408-427.
Nasu, Makoto et al. (2012). “Robust Formation and Maintenance of Continuous Stratified Cortical Neuroepithelium by Laminin-Containing Matrix in Mouse ES Cell Culture”. In: PLOS ONE 7.12, pp. 13-14.
Nelson, Branden R. et al. (May 2013). “Dynamic Interactions between Intermediate Neurogenic Progenitors and Radial Glia in Embryonic Mouse Neocortex: Potential Role in Dll1-Notch Signaling”. In: Journal of Neuroscience 33.21, pp. 9122-9139.
Nielsen, Corinne M. and Susan M. Dymecki (April 2010). “Sonic hedgehog is required for vascular outgrowth in the hindbrain choroid plexus”. In: Developmental Biology 340.2, pp. 430-437.
Noort, Rebecca J. et al. (2024). “Apically localized PANX1 impacts neuroepithelial expansion in human cerebral organoids”. In: Cell Death Discovery 10.1, pp. 1-18.
Nour, Matthew M., Yunzhe Liu, and Raymond J. Dolan (August 2022). “Functional neuroimaging in psychiatry and the case for failing better”. In: Neuron 110.16, pp. 2524-2544.
Nourse, Jamison L. et al. (October 2022). “Mechanotransduction by Membrane Proteins Piezo1 regulates cholesterol biosynthesis to influence neural stem cell fate during brain development”. In: Journal of General Physiology 154.10.
Novotna, Sarka et al. (November 2024). “Linking planar polarity signalling to actomyosin contractility during vertebrate neurulation”. In: Open Biology 14.11.
O'Laughlin, Richard et al. (June 2025). “Bioengineering tools for next-generation neural organoids”. In: Current Opinion in Neurobiology 92, p. 103011.
Oda, Yoshio and Isao Nakanishi (February 1987). “Ultrastructure of the caudal portion of the fourth ventricular roof in the mouse”. In: Journal of Comparative Neurology 256.2, pp. 299-307.
Ohtsuka, Toshiyuki and Ryoichiro Kageyama (February 2021). “Hes1 overexpression leads to expansion of embryonic neural stem cell pool and stem cell reservoir in the postnatal brain”. In: Development (Cambridge) 148.4.
Okamoto, Mayumi, Takaki Miyata, et al. (2016). “Cell-cycle-independent transitions in temporal identity of mammalian neural progenitor cells”. In: Nature Communications 7.
Okamoto, Mayumi, Takashi Namba, et al. (September 2013). “TAG-1-assisted progenitor elongation streamlines nuclear migration to optimize subapical crowding”. In: Nature Neuroscience 2013 16:11, pp. 1556-1566.
Otani, Tomoki et al. (April 2016). “2D and 3D Stem Cell Models of Primate Cortical Development Identify Species-Specific Differences in Progenitor Behavior Contributing to Brain Size”. In: Cell Stem Cell 18.4, pp. 467-480.
Ozair, Mohammad Zeeshan, Chris Kintner, and Ali H. Brivanlou (July 2013). “Neural induction and early patterning in vertebrates”. In: Wiley Interdisciplinary Reviews: Developmental Biology 2.4, pp. 479-498.
Pacheco, Mary et al. (1986). “Quantification of the initial phases of rapid brain enlargement in the chick embryo”. In: American Journal of Anatomy 175.4, pp. 403-411.
Pagliaro, Anna, Francesco Andreatta, et al. (February 2025). “Generation of human fetal brain organoids and their CRISPR engineering for brain tumor modeling”. In: Nature Protocols 2025, pp. 1-38.
Pagliaro, Anna, Benedetta Artegiani, and Delilah Hendriks (2025). “Emerging approaches to enhance human brain organoid physiology”. In: Trends in Cell Biology 0.0, p. 17.
Pagliaro, Anna, Roxy Finger, et al. (2023). “Temporal morphogen gradient-driven neural induction shapes single expanded neuroepithelium brain organoids with enhanced cortical identity”. In: Nature Communications 14.1.
Pai, Yun Jin et al. (2012). “Epithelial fusion during neural tube morphogenesis”. In: Birth Defects Research Part A-Clinical and Molecular Teratology 94.10, pp. 817-823.
Paolino, Annalisa et al. (September 2023). “Non-uniform temporal scaling of developmental processes in the mammalian cortex”. In: Nature Communications 2023 14:1 14.1, pp. 1-17.
Park, JiSoo et al. (October 2022). “Self-organizing in vitro mouse neural tube organoids mimic embryonic development”. In: Development 149.20, dev201052.
Park, Sangjun et al. (January 2025). “Long-term tracking of neural and oligodendroglial development in large-scale human cerebral organoids by noninvasive volumetric imaging”. In: Scientific Reports 2025 15:1, pp. 1-17.
Parvas, Maryam, Carolina Parada, and David Bueno (September 2008). “A blood-CSF barrier function controls embryonic CSF protein composition and homeostasis during early CNS development”. In: Developmental Biology 321.1, pp. 51-63.
Patel, Devan et al. (August 2024). “Microinstrumentation for Brain Organoids”. In: Advanced Healthcare Materials 13.21, p. 2302456.
Patten, B. M. (1952). “Overgrowth of the neural tube in young human embryos.” In: The Anatomical record 113.4, pp. 381-393.
Paulsen, Bruna et al. (2022). “Autism genes converge on asynchronous development of shared neuron classes”. In: Nature 602.7896, pp. 268-273.
Pellegrini, Laura et al. (2020). “Human CNS barrier-forming organoids with cerebrospinal fluid production”. In: Science 369.6500, pp. 0-13.
Perrottelli, Andrea et al. (March 2024). “Advances in the understanding of the pathophysiology of schizophrenia and bipolar disorder through induced pluripotent stem cell models”. In: Journal of Psychiatry and Neuroscience 49.2, E109-E125.
Pexieder, T. and R. Jelinek (1970). “Pressure of the CSF and the Morphogenesis of the CNS: II. Pressure Necessary for Normal Development of Brain Vesicles”. In: Folia Morphologica 18.2, pp. 181-192.
Pigoni, Martina et al. (2023). “Cell-type specific defects in PTEN-mutant cortical organoids converge on abnormal circuit activity”. In: Human Molecular Genetics 00. June, pp. 1-14.
Pinheiro, Diana and Yohanns Bellaiche (October 2018). “Mechanical Force-Driven Adherens Junction Remodeling and Epithelial Dynamics”. In: Developmental Cell 47.1, pp. 3-19.
Pinson, Anneline et al. (September 2022). “Human TKTL1 implies greater neurogenesis in frontal neocortex of modern humans than Neanderthals”. In: Science 377.6611.
Pollen, Alex and Arnold Kriegstein (2023). “Neocortical Neurogenesis in Development and Evolution”. In: ed. by Wieland Huttner. John Wiley & Sons. Chap. Diversity and Evolution of Human Cortical Progenitor Cell Types.
Prastowo, Aishah et al. (December 2016). “Biocompatibility of fluids for multiphase drops-in-drops microfluidics”. In: Biomedical Microdevices 18.6, pp. 1-9.
Punovuori, Karolina et al. (November 2019). “N-cadherin stabilises neural identity by dampening anti-neural signals”. In: Development (Cambridge) 146.21.
Qian, Xuyu et al. (2020). “Sliced Human Cortical Organoids for Modeling Distinct Cortical Layer Formation”. In: Cell Stem Cell 26.5, pp. 766-781.
Quadrato, Giorgia and Paola Arlotta (2017). “Present and future of modeling human brain development in 3D organoids”. In: Current Opinion in Cell Biology 49, pp. 47-52.
Quadrato, Giorgia, Tuan Nguyen, et al. (2017). “Cell diversity and network dynamics in photosensitive human brain organoids”. In: Nature 545.7652, pp. 48-53.
Rahman, Abdel et al. (2023). “Targeted mechanical stimulation via magnetic nanoparticles guides in vitro tissue development”.
Rakic, Pasko (1995). “A small step for the cell, a giant leap for mankind: a hypothesis of neocortical expansion during evolution”. In: Trends in Neurosciences 18.9, pp. 383-388.
Rammensee, Sebastian et al. (February 2017). “Dynamics of Mechanosensitive Neural Stem Cell Differentiation”. In: Stem Cells 35.2, pp. 497-506.
Ranga, Adrian et al. (2017). “Neural tube morphogenesis in synthetic 3D microenvironments”. In: Proceedings of the National Academy of Sciences of the United States of America 113.44, E3163.
Rasmussen, Martin Kaag, Humberto Mestre, and Maiken Nedergaard (2022). “Fluid transport in the brain”. In: Physiological Reviews 102.2, pp. 1025-1151.
Reiner, Orly, Arpan Parichha, and Tamar Sapir (2021). “Modeling human neuronal migration deficits in 3D”. In: Current Opinion in Neurobiology 66, pp. 30-36.
Rodriguez-Gatica, Juan Eduardo et al. (August 2022). “Imaging three-dimensional brain organoid architecture from meso- to nanoscale across development”. In: Development 149.20.
Romani, Patrizia et al. (November 2020). “Crosstalk between mechanotransduction and metabolism”. In: Nature Reviews Molecular Cell Biology 2020 22:1, pp. 22-38.
Romero, Delfina M., Nadia Bahi-Buisson, and Fiona Francis (2018). “Genetics and mechanisms leading to human cortical malformations”. In: Seminars in Cell and Developmental Biology 76, pp. 33-75.
Rose, F. Clifford (July 2009). “Cerebral Localization in Antiquity”. In: Journal of the History of the Neurosciences 18.3, pp. 239-247.
Rosenbaum, Stacy and Christopher W. Kuzawa (September 2023). “The promise of great apes as model organisms for understanding the downstream consequences of early life experiences”. In: Neuroscience & Biobehavioral Reviews 152, p. 105240.
Roth, Gerhard and Ursula Dicke (January 2019). “Origin and evolution of human cognition”. In: Progress in Brain Research 250, pp. 285-316.
Roth, Julien G. et al. (2021). “Advancing models of neural development with biomaterials”. In: Nature Reviews Neuroscience 22.10, pp. 593-615.
Rothenbucher, Theresa S. P. et al. (2021). “Next generation human brain models: Engineered flat brain organoids featuring gyrification”. In: Biofabrication 13.1.
Rousso, David L. et al. (April 2012). “Foxp-Mediated Suppression of N-Cadherin Regulates Neuroepithelial Character and Progenitor Maintenance in the CNS”. In: Neuron 74.2, pp. 314-330.
Ruiz-Reig, Nuria et al. (May 2017). “Lateral Thalamic Eminence: A Novel Origin for mGluR 1/Lot Cells”. In: Cerebral Cortex 27.5, pp. 2841-2856.
Russo, Fabiele B. et al. (March 2021). “ZIKV Teratogenesis: Clinical Findings in Humans, Mechanisms and Experimental Models”. In: Frontiers in Virology 1, p. 775361.
Saito, Kanako et al. (January 2018). “Neural Progenitor Cells Undergoing Yap/Tead-Mediated Enhanced Self-Renewal Form Heterotopias More Easily in the Diencephalon than in the Telencephalon”. In: Neurochemical research 43.1, pp. 171-180.
Sauer, F. C. (December 1936). “The interkinetic migration of embryonic epithelial nuclei”. In: Journal of Morphology 60.1, pp. 1-11.
Saunders, Norman (1992). “Barriers and Fluids of the Eye and Brain”. In: Palgrave, London. Chap. Development of the Blood-Brain Barrier to Macromolecules.
Saunders, Norman et al. (2023). “the Choroid Plexus: a Missing Link in Our Understanding of Brain Development and Function”. In: Physiological Reviews 103.1, pp. 919-956.
Schnell, Alexandra K. et al. (February 2021). “How intelligent is a cephalopod? Lessons from comparative cognition”. In: Biological Reviews 96.1, pp. 162-178.
Schwarz, Niklas et al. (September 2017). “Human Cerebrospinal fluid promotes long-term neuronal viability and network function in human neocortical organotypic brain slice cultures”. In: Scientific Reports 2017 7:1, pp. 1-12.
Schwerk, Christian and Horst Schroten (September 2024). “In vitro models of the choroid plexus and the blood-cerebrospinal fluid barrier: advances, applications, and perspectives”. In: Human Cell 37.5, pp. 1235-1242.
Scuderi, Soraya et al. (2021). “Cell-to-Cell Adhesion and Neurogenesis in Human Cortical Development: A Study Comparing 2D Monolayers with 3D Organoid Cultures”. In: Stem Cell Reports 16.2, pp. 264-280.
Seifert, Udo (1997). Configurations of fluid membranes and vesicles. Vol. 46. 1, pp. 13-137.
Sekine, Katsutoshi, Ken Ichiro Kubo, and Kazunori Nakajima (September 2014). “How does Reelin control neuronal migration and layer formation in the developing mammalian neocortex?” In: Neuroscience Research 86, pp. 50-58.
Senovilla-Ganzo, Rodrigo and Fernando Garcia-Moreno (May 2024). “The Phylotypic Brain of Vertebrates, from Neural Tube Closure to Brain Diversification”. In: Brain Behavior and Evolution 99.1, pp. 45-68.
Shcholok, Tetiana and Eftekhar Eftekharpour (February 2022). “Cre-recombinase systems for induction of neuron-specific knockout models: a guide for biomedical researchers”. In: Neural Regeneration Research 18.2, p. 273.

Shekdar, Karuna (June 2011). “Posterior Fossa Malformations”. In: Seminars in Ultrasound, CT and MRI 32.3, pp. 228-241.
Shi, Riyi and RIchard Borgens (1994). “Embryonic Neuroepithelial Sodium Transport, the Resulting Physiological Potential, and Cranial Development”. In: Developmental Biology 165, pp. 105-116.
Shibata, Mikihito et al. (October 2021). “Regulation of prefrontal patterning and connectivity by retinoic acid”. In: Nature 2021 598:7881 598.7881, pp. 483-488.
Shim, Gawoon et al. (April 2024). “Bioelectric stimulation controls tissue shape and size”. In: Nature Communications 2024 15:1 15.1, pp. 1-17.
Shinoda, Tomoyasu et al. (April 2018). “Elasticity-based boosting of neuroepithelial nucleokinesis via indirect energy transfer from mother to daughter”. In: PLOS Biology 16.4, e2004426.
Shiraishi, N. et al. (2015). “Morphology and morphometry of the human embryonic brain: A three-dimensional analysis”. In: NeuroImage 115, pp. 96-103.
Siegenthaler, Julie A., Amir M. Ashique, et al. (2009). “Retinoic Acid from the Meninges Regulates Cortical Neuron Generation”. In: Cell 139.3, pp. 597-609. Siegenthaler, Julie A. and Samuel J. Pleasure (2011). “We have got you ‘covered’: How the meninges control brain development”. In: Current Opinion in Genetics and Development 21.3, pp. 249-255.
Simeone, Antonio et al. (1992). “Nested expression domains of four homeobox genes in developing rostral brain”. In: Nature 1992 358:6388 358.6388, pp. 687-690.
Singh, Jasmeet (2024). “Cerebral organoid precursor heterogeneity is organized into nonrandom positionally dependent anatomical compartments random positionally dependent anatomical compartments”. PhD thesis. University of Toronto (Canada).
Sivitilli, Adam et al. (2020). “Robust production of uniform human cerebral organoids from pluripotent stem cells”. In: Life Science Alliance 3.5, pp. 1-10.
Skylar-Scott, Mark A. et al. (2019). “Voxelated soft matter via multimaterial multinozzle 3D printing”. In: Nature 575.7782, pp. 330-335.
Snyder, Jason S. (March 2019). “Recalibrating the Relevance of Adult Neurogenesis”. In: Trends in Neurosciences 42.3, pp. 164-178.
Solmi, Marco et al. (July 2023). “Incidence, prevalence, and global burden of schizophrenia-data, with critical appraisal, from the Global Burden of Disease (GBD) 2019”. In: Molecular Psychiatry 2023 28:12 28.12, pp. 5319-5327.
Spear, Philip C. and Carol A. Erickson (2012). “Interkinetic nuclear migration: A mysterious process in search of a function”. In: Development Growth and Differentiation 54.3, pp. 306-316.
Spuy, Marise van der et al. (2023). “The cellular dynamics of neural tube formation”. In: Biochemical Society Transactions 51.1, pp. 343-352.
Stepien, Barbara K., Samir Vaid, and Wieland Huttner (May 2021). “Length of the Neurogenic Period—A Key Determinant for the Generation of Upper-Layer Neurons During Neocortex Development and Evolution”. In: Frontiers in Cell and Developmental Biology 9, p. 676911.
Stiles, Joan and Terry Jernigan (December 2010). “The Basics of Brain Development”. In: Neuropsychology Review 20.4, p. 327.
Stoufflet, Julie, Sylvia Tielens, and Laurent Nguyen (June 2023). “Shaping the cerebral cortex by cellular crosstalk”. In: Cell 186.13, pp. 2733-2747.
Stringer, Carsen et al. (December 2020). “Cellpose: a generalist algorithm for cellular segmentation”. In: Nature Methods 2020 18:1 18.1, pp. 100-106.
Stukel, Jessica M. and Rebecca Kuntz Willits (January 2016). “Mechanotransduction of Neural Cells Through Cell-Substrate Interactions”. In: home.liebertpub.com/teb 22.3, pp. 173-182.
Su, Yi Ching et al. (May 2022). “YAP maintains the production of intermediate progenitor cells and upper-layer projection neurons in the mouse cerebral cortex”. In: Developmental Dynamics 251.5, pp. 846-863.
Su, Yue et al. (March 2025). “Bovine Formative Embryonic Stem Cell Plasticity in Embryonic and Extraembryonic Differentiation”. In: bioRxiv, p. 2024.12.28.630627.
Subramanian, Aravind et al. (October 2005). “Gene set enrichment analysis: A knowledge-based approach for interpreting genome-wide expression profiles”. In: Proceedings of the National Academy of Sciences of the United States of America 102.43, pp. 15545-15550.
Subramanian, Lakshmi et al. (2017). “Dynamic behaviour of human neuroepithelial cells in the developing forebrain”. In: Nature Communications 8.
Sutcliffe, Magdalena and Madeline Lancaster (2023). “Neocortical Neurogenesis in Development and Evolution”. In: ed. by Wieland Huttner. John Wiley & Sons. Chap. Understanding Human Forebrain Morphogenesis and Early Expansion Using Organoids.
Suzuki, Ikuo K. and Pierre Vanderhaeghen (September 2015). “Is this a brain which I see before me? Modeling human neural development with pluripotent stem cells”. In: Development 142.18, pp. 3138-3150.
Takla, Taylor N. et al. (July 2023). “A Shared Pathogenic Mechanism for Valproic Acid and SHROOM3 Knockout in a Brain Organoid Model of Neural Tube Defects”. In: Cells 12.13, p. 1697.
Tanaka, Yoshiaki et al. (February 2020). “Synthetic Analyses of Single-Cell Transcriptomes from Multiple Brain Organoids and Fetal Brain”. In: Cell Reports 30.6, pp. 1682-1689.
Tang, Xiaowei et al. (October 2023). “Volumetric compression by heterogeneous scaffold embedding promotes cerebral organoid maturation and does not impede growth”. In: Cell Systems 14.10, pp. 872-882.
Terashima, Mei et al. (2021). “Early development of the cortical layers in the human brain”. In: Journal of Anatomy 239.5, pp. 1039-1049.
Terry, Bethany K. and Seonhee Kim (2022). “The role of Hippo-YAP/TAZ signaling in brain development”. In: Developmental Dynamics 251.10, pp. 1644-1665.
Thomas, Carson H. et al. (February 2002). “Engineering gene expression and protein synthesis by modulation of nuclear shape”. In: Proceedings of the National Academy of Sciences 99.4, pp. 1972-1977.
Tidball, Andrew M. et al. (2023). “Deriving early single-rosette brain organoids from human pluripotent stem cells”. In: Stem Cell Reports 18.12, pp. 2498-2514.
Tirosh, Itay et al. (April 2016). “Dissecting the multicellular ecosystem of metastatic melanoma by single-cell RNA-seq”. In: Science 352.6282, pp. 189-196.
Toma, Kenichi et al. (September 2014). “The Timing of Upper-Layer Neurogenesis Is Conferred by Sequential Derepression and Negative Feedback from Deep-Layer Neurons”. In: Journal of Neuroscience 34.39, pp. 13259-13276.
Tsai, Huei Mei, Beatrice Garber, and Luis Larramendi (1981). “3H-Thymidine autoradiographic analysis of telencephalic histogenesis in the chick embryo: I. Neuronal birthdates of telencephalic compartments in situ”. In: Journal of Comparative Neurology 198.2, pp. 275-292.
Tsai, Jin Wu et al. (October 2010). “Kinesin 3 and cytoplasmic dynein mediate interkinetic nuclear migration in neural stem cells”. In: Nature Neuroscience 2010 13:12, pp. 1463-1471
Uhler, Caroline and G. V. Shivashankar (October 2017). “Regulation of genome organization and gene expression by nuclear mechanotransduction”. In: Nature Reviews Molecular Cell Biology 2017 18:12, pp. 717-727.
Uitterlinden, E. J. et al. (2008). “Glucosamine increases hyaluronic acid production in human osteoarthritic synovium explants”. In: BMC Musculoskeletal Disorders 9, pp. 1-7.
Uzquiano, Ana and Paola Arlotta (2022). “Brain organoids: the quest to decipher human specific features of brain development”. In: Current Opinion in Genetics and Development 75, p. 101955.
Uzquiano, Ana, Ivan Gladwyn-Ng, et al. (2018). “Cortical progenitor biology: key features mediating proliferation versus differentiation”. In: Journal of Neurochemistry 146.5, pp. 500-525.
Uzquiano, Ana, Amanda J Kedaigle, et al. (2022). “Proper acquisition of cell class identity in organoids allows definition of fate specification programs of the human cerebral cortex”. In: Cell, pp. 3770-3788.
Valamparamban, Ghanim Fajish and Pauline Speder (December 2023). “Homemade: building the structure of the neurogenic niche”. In: Frontiers in Cell and Developmental Biology 11, p. 1275963.
Van den Ameele, Jelle et al. (June 2014). “Thinking out of the dish: What to learn about cortical development using pluripotent stem cells”. In: Trends in Neurosciences 37.6, pp. 334-342.
Varadarajan, Saranyaraajan, Rachel E. Stephenson, and Ann L. Miller (November 2019). “Multiscale dynamics of tight junction remodeling”. In: Journal of Cell Science 132.22.
Veeraval, Lenin, Conor J. O'Leary, and Helen Cooper (2020). “Adherens Junctions: Guardians of Cortical Development”. In: Frontiers in Cell and Developmental Biology 8. January, pp. 1-17.
Velasco, Silvia, Bruna Paulsen, and Paola Arlotta (2019). “Highly reproducible human brain organoids recapitulate cerebral cortex cellular diversity”. In: Protocol Exchange, pp. 1-17.
Vertesy, Abel et al. (September 2022). “Gruffi: an algorithm for computational removal of stressed cells from brain organoid transcriptomic datasets”. In: The EMBO Journal 41.17.
Verwer, Ronald W. H. et al. (July 2015). “Injury Response of Resected Human Brain Tissue in Vitro”. In: Brain Pathology 25.4, pp. 454-468.
Wallace, Jenelle L. and Alex Pollen (November 2023). “Human neuronal maturation comes of age: cellular mechanisms and species differences”. In: Nature Reviews Neuroscience 2023 25:1 25.1, pp. 7-29.
Wang, Jing et al. (April 2021). “EZH2 Regulates the Correlation between Skin Regeneration and the Duration of Mechanical Stretch”. In: Journal of Investigative Dermatology 141.4, pp. 894-902.
Wang, Kyu Chang et al. (April 1997). “Functional Opening of the Fourth Ventricular Outlet in C57BL/6J and Delayed Splotch Mouse Embryos”. In: Pediatric Neurosurgery 27.4, pp. 176-181.
Wang, Yueqi et al. (2022). “Modeling human telencephalic development and autism-associated SHANK3 deficiency using organoids generated from single neural rosettes”. In: Nature Communications 13.1.
Watanabe, Keisuke et al. (September 2023). “Spatiotemporal expression patterns of R-spondins and their receptors, Lgrs, in the developing mouse telencephalon”. In: Gene Expression Patterns 49, p. 119333.
Watanabe, Kiichi et al. (February 2005). “Directed differentiation of telencephalic precursors from embryonic stem cells”. In: Nature Neuroscience 2005 8:3 8.3, pp. 288-296.
Watanabe, Yuto, Takumi Kawaue, and Takaki Miyata (July 2018). “Differentiating cells mechanically limit the interkinetic nuclear migration of progenitor cells to secure apical cytogenesis”. In: Development (Cambridge) 145.14.
Wen, Han et al. (2019). “Alginate Hydrogel Modified with a Ligand Interacting with α3β1 Integrin Receptor Promotes the Differentiation of 3D Neural Spheroids toward Oligodendrocytes in Vitro”. In: ACS Applied Materials & Interfaces 11, pp. 5821-5833.
Whish, Sophie et al. (February 2015). “The inner csf-brain barrier: Developmentally controlled access to the brain via intercellular junctions”. In: Frontiers in Neuroscience 9, February, p. 124778.
Wilson, Doris Burda (1972). “Effects of Embryonic Overgrowth on the Avian Optic Tectum”. In: American Journal of Anatomy 135.4, pp. 549-560. doi: 10.1002/aja.1001350408.
(1974). “The cell cycle of ventricular cells in the overgrown optic tectum”. In: Brain Research 69.1, pp. 41-48.
(August 1978). “Sequence of neuron origin in experimentally distorted brains of chick embryos”. In: Journal of Comparative Neurology 180.3, pp. 501-508.
Wolf, Kayla J. et al. (2022). “Biomanufacturing human tissues via organ building blocks”. In: Cell Stem Cell 29.5, pp. 667-677.
Wu, Quan, Merve Bilgic, and Fumio Matsuzaki (2023). “Neocortical Neurogenesis in Development and Evolution”. In: ed. by Wieland Huttner. John Wiley & Sons. Chap. Temporal Scaling: A Link between Conserved Neurogenic Processes and Differential Size in Mammalian Cortical Development.
Wu, Yu and Richard H Finnell (2021). “Organoids as a new model system to study neural tube defects”. In: February, pp. 1-11.
Xia, Bingzhao, Kaspars Krutkramelis, and John Oakey (July 2016). “Oxygen-Purged Microfluidic Device to Enhance Cell Viability in Photopolymerized PEG Hydrogel Microparticles”. In: Biomacromolecules 17.7, pp. 2459-2465.
Xu, Gang et al. (July 2010). “Axons pull on the brain, but tension does not drive cortical folding”. In: Journal of Biomechanical Engineering 132.7.
Xue, Xufeng et al. (February 2024). “A patterned human neural tube model using microfluidic gradients”. In: Nature 2024 628:8007 628.8007, pp. 391-399.
Yang, Ming, Hua Xiang, and Guoshun Luo (June 2024). “Targeting focal adhesion kinase (FAK) for cancer therapy: FAK inhibitors, FAK-based dual-target inhibitors and PROTAC degraders”. In: Biochemical Pharmacology 224, p. 116246.
Yang, Xiaojun et al. (September 2009). “Stepwise Maturation of Apicobasal Polarity of the Neuroepithelium Is Essential for Vertebrate Neurulation”. In: Journal of Neuroscience 29.37, pp. 11426-11440.
YiJun, Xie et al. (2024). “Global, Regional, and National Burdens of Developmental Disabilities in Children and Adolescents from 1990 to 2021: A Trend Analysis Insights from GBD 2021 Data”.
Ying, Qi Long et al. (January 2003). “Conversion of embryonic stem cells into neuroectodermal precursors in adherent monoculture”. In: Nature Biotechnology 2003 21:2 21.2, pp. 183-186.
Yokota, Yukako et al. (2009). “The Adenomatous Polyposis Coli Protein Is an Essential Regulator of Radial Glial Polarity and Construction of the Cerebral Cortex”. In: Neuron 61.1, pp. 42-56.
Yoon, Ki Jun et al. (May 2008). “Mind Bomb 1-Expressing Intermediate Progenitors Generate Notch Signaling to Maintain Radial Glial Cells”. In: Neuron 58.4, pp. 519-531.
Yoon, Se Jin et al. (2019). “Reliability of human cortical organoid generation”. In: Nature Methods 16.1, pp. 75-78. issn: 154871
Yousuf, Mujeeb et al. (2025). “Advancing Brain Organoid Electrophysiology: Minimally Invasive Technologies for Comprehensive Characterization”. In: Advanced Materials Technologies, p. 2401585.
Zappaterra, Mauro D. et al. (2007). “A comparative proteomic analysis of human and rat embryonic cerebrospinal fluid”. In: Journal of Proteome Research 6.9, pp. 3537-3548.
Ze ̆cevic, N. (March 1993). “Cellular composition of the telencephalic wall in human embryos”. In: Early Human Development 32.2-3, pp. 131-149.
Zhang, Andrew (2022). “Brain development outside the embryonic niche”. PhD thesis. Harvard University.
Zhang, Jianing et al. (March 2010). “Cortical Neural Precursors Inhibit Their Own Differentiation via N-Cadherin Maintenance of β-Catenin Signaling”. In: Developmental Cell 18.3, pp. 472-479.
Zhang, Jingjing et al. (2010). “Establishment of a neuroepithelial barrier by Claudin5a is essential for zebrafish brain ventricular lumen expansion”. In: Proceedings of the National Academy of Sciences of the United States of America 107.4, pp. 1425-1430.
Zhang, Lili and Xiangyun Wei (June 2022). “The Roles of Par3, Par6, and aPKC Polarity Proteins in Normal Neurodevelopment and in Neurodegenerative and Neuropsychiatric Disorders”. In: Journal of Neuroscience 42.24, pp. 4774-4793.
Zhang, Runrui et al. (October 2023). “Neurogenesis in primates versus rodents and the value of non-human primate models”. In: National Science Review 10.11.
Zhang, Shuchen et al. (April 2019). “OCT4 and PAX6 determine the dual function of SOX2 in human ESCs as a key pluripotent or neural factor”. In: Stem Cell Research and Therapy 10.1, pp. 1-14.
Zhang, Xiao Zuo et al. (May 2022). “Folic Acid Rescues Valproic Acid-Induced Morphogenesis Inhibition in Neural Rosettes Derived From Human Pluripotent Stem Cells”. In: Frontiers in Cellular Neuroscience 16, p. 888152.
Zhang, Xiaoqing et al. (July 2010). “Pax6 is a human neuroectoderm cell fate determinant”. In: Cell Stem Cell 7.1, pp. 90-100.
Zhang, Z. et al. (December 2024). “Mapping fetal brain development of 10 weeks gestational age with 9.4 T postmortem MRI and histological sections”. In: American Journal of Neuroradiology, ajnr.A8595
Zhou, Chenlin et al. (January 2016). “Comprehensive profiling reveals mechanisms of SOX2-mediated cell fate specification in human ESCs and NPCs”. In: Cell Research 2016 26:2 26.2, pp. 171-189.
Zhou, Yi, Hongjun Song, and Guo li Ming (July 2023). “Genetics of human brain development”. In: Nature Reviews Genetics 2023 25:1 25.1, pp. 26-45.
Zhu, Yu et al. (2013). “Three-Dimensional Neuroepithelial Culture from Human Embryonic Stem Cells and Its Use for Quantitative Conversion to Retinal Pigment Epithelium”. In: PLOS ONE 8.1.
Zywitza, Vera et al. (November 2022). “Induced pluripotent stem cells and cerebral organoids from the critically endangered Sumatran rhinoceros”. In: iScience 25.11.

What is claimed is:

1. A method of controlling neurogenic differentiation of an organoid comprising modifying the intraluminal pressure of an organoid, thereby controlling the neurogenic differentiation of the organoid.

2. The method of claim 1, wherein the organoid is a neuroepithelial organoid.

3. The method of claim 1, wherein the intraluminal pressure of the organoid is increased by microinjecting fluid into one or more lumens of the organoid, thereby producing an inflated organoid.

4. The method of claim 3, wherein the fluid is selected from the group consisting of: silicone oil, hydroxyfluoroether, or a solution of hyaluronic acid in saline.

5. The method of claim 3, wherein the fluid is injected into the lumen every 2 to 5 days for a period of up to 45 days.

6. The method of claim 3, wherein the fluid is injected into the lumen every 2 to 5 days for a period of up to 90 days.

7. The method of claim 3, wherein the fluid is injected into the lumen at a time point before rosettes appear in the organoid.

8. The method of claim 3, wherein increasing intraluminal pressure in the organoid inhibits neurogenic differentiation.

9. The method of claim 3, wherein increasing intraluminal pressure in the organoid promotes or prolongs stem cell proliferation.

10. The method of claim 3, wherein the inflated organoid exhibits one or more of formation of single-rosette neural organoids greater than 500 μm in diameter, delayed appearance of ectopic additional rosettes, and stable maintenance of single-rosette organoids for up to 50 days.

11. The method of claim 3, wherein the inflated organoid exhibits one or more of uniform biomimetic radial organization, improved quality and longevity of tissue architecture, uniform neural induction, having a single continuous ventricular zone, having uniform radial glial scaffold spanning the entire tissue, and lack of hypoxia.

12. The method of claim 3, further comprising reducing intraluminal pressure of the inflated organoid, thereby producing a deflated organoid.

13. The method of claim 11, wherein reduction of intraluminal pressure results in the activation of neurogenic differentiation of the deflated organoid.

14. An inflated single-lumen cortical organoid, wherein the lumen of the cortical organoid is injected with a fluid to increase lumen pressure.

15. The organoid of claim 14, wherein the fluid is selected from the group consisting of: silicone oil, hydroxyfluoroether, or a solution of hyaluronic acid in saline.

16. The organoid of claim 14, wherein the organoid maintains tissue architecture resembling that of the developing mammalian neocortex for up to 1.5 months.

17. The organoid of claim 14, wherein neurogenic differentiation of the organoid is inhibited.

18. The organoid of claim 14, wherein the organoid comprises inflated cortical progenitors enriched for genes associated with DNA replication.

19. The organoid of claim 14, wherein the organoid comprises inflated projection neurons enriched in GO terms related to mature neuronal function.

20. The organoid of claim 14, wherein the organoid exhibits one or more of: formation of single-rosette neural organoids greater than 500 μm in diameter, delayed appearance of ectopic additional rosettes, and stable maintenance of single-rosette organoids for up to 50 days.
